(19) United States
(12) Patent Application Publication
(10) Pub. No.: US 2004/0009489 A1
(43) Pub. Date: Jan. 15, 2004

(54) Classification of lung carcinomas using gene expression analysis

(76) Inventors: Todd R. Golub, Newton, MA (US); Matthew Meyerson, Concord, MA (US); Arindam Bhattacharjee, Andover, MA (US); Jane Staunton, Cambridge, MA (US)

Correspondence Address: TESTA, HURWITZ & THIBEAULT, LLP, HIGH STREET TOWER, 125 HIGH STREET, BOSTON, MA, 02110, US

(21) Appl. No.: 10/259,233

(22) Filed: Sep. 27, 2002

Related U.S. Application Data
(60) Provisional application No. 60/325,962, filed on Sep. 28, 2001.

(51) Int. Cl. C12Q 1/68

(52) U.S. Cl. 435/6

(57) ABSTRACT

The invention provides a molecular taxonomy of lung carcinoma, the leading cause of cancer death in the United States and worldwide. Oligonucleotide microarrays were used to analyze mRNA expression levels corresponding to 12,600 transcript sequences in 186 lung tumor samples, including 139 adenocarcinomas resected from the lung. Hierarchical and probabilistic clustering of expression data defined distinct subclasses of lung adenocarcinoma. Among these were tumors with high relative expression of neuroendocrine genes and of type II pneumocyte genes, respectively. Retrospective analysis revealed a less favorable outcome for the adenocarcinomas with neuroendocrine gene expression. The diagnostic potential of expression profiling is emphasized by its ability to discriminate primary lung adenocarcinomas from metastases of extrapulmonary origin. These results suggest that integration of expression profile data with clinical parameters could aid in diagnosis of lung cancer patients.

### RELATED APPLICATIONS

This application claims priority to, and the benefit of, Provisional Patent Application U.S. S No. 60/325/962 filed on Sep. 28, 2001, the entire disclosure of which is incorporated by reference herein.

### GOVERNMENT SUPPORT

The invention was supported, in whole or in part, by grant U01 CA84995 from the National Cancer Institute. The Government has certain rights in the invention.

### FIELD OF THE INVENTION

In general, the invention relates to a gene expression based classification of lung cancer and a sub-classification of lung adenocarcinoma. This classification serves as a step towards a new molecular taxonomy of lung tumors and demonstrates the power of gene expression profiling in lung cancer diagnosis.

### BACKGROUND

Carcinoma of the lung claims more than 150,000 lives every year in the United States, thus exceeding the combined mortality from breast, prostate and colorectal cancers. Current lung cancer classification is based on clinicopathological features. Lung carcinomas are usually classified as small cell lung carcinomas (SCLC) or non-small cell lung carcinomas (NSCLC). Neuroendocrine features, defined by microscopic morphology and immuno-histochemistry, are hallmarks of the high-grade SCLC and large cell neuroendocrine tumors and of intermediate/low-grade carcinoid tumors. NSCLC is histopathologically and clinically distinct from SCLC, and is further subcategorized as adenocarcinomas, squamous cell carcinomas, and large cell carcinomas, of which adenocarcinomas are the most common.

The histopathological sub-classification of lung adenocarcinoma is challenging. In one study, independent lung pathologists agreed on lung adenocarcinoma sub-classification in only 41% of cases. However, a favorable prognosis for bronchioloalveolar carcinoma (BAC), a histological sub-class of lung adenocarcinoma, argues for refining such distinctions. In addition, metastases of non-lung origin can be difficult to distinguish from lung adenocarcinomas.

Therefore, there is a need in the art for methods and compositions that are useful to distinguish cancer of lung origin from metastases of non-lung origin, and to distinguish different types of lung cancer.

### SUMMARY

The development of microarray methods for large-scale analysis of gene expression makes it possible to search systematically for molecular markers of cancer classification and outcome prediction in a variety of tumor types. Currently, the only effective prognostic indicator for NSCLC in clinical use is surgical-pathological staging. However, according to the invention, the simultaneous analysis of a large number of independent clinical markers offers a powerful adjunct approach in surgical-pathological staging.

According to the invention, a comprehensive gene expression analysis of human lung tumors identified distinct lung adenocarcinoma sub-classes that were reproducibly generated across different cluster methods. Notably, the C2 adenocarcinoma subclass, defined by neuroendocrine gene expression, is associated with a less favorable outcome, while the C4 group appears to be associated with a more favorable outcome.

Hierarchical clustering methods offer a powerful approach for class discovery, but are less useful for determining confidence for the classes discovered. In one aspect of the invention, a bootstrap probabilistic clustering is combined with the hierarchical method to measure the strength of sample-sample association, thereby defining cluster membership with greater confidence.

Although adenocarcinomas with neuroendocrine features have been reported, unique markers that precisely define such tumors have not been described. In another aspect of the invention, putative neuroendocrine markers, for example, kallikrein 11, that discriminate the C2 tumors from all other lung tumors, are identified. In one embodiment, this marker, which is related to the vasodepressor renal kallikrein, is of clinical interest given the observation of orthostatic hypotension in some lung cancer patients.

In a further aspect of the invention, putative metastases of extra-pulmonary origin with non-lung expression signatures were discovered among presumed lung adenocarcinomas. According to the invention, gene expression analysis can serve as a diagnostic tool to confirm and identify metastases to the lung.

In one embodiment, the invention provides lung specific marker arrays. In another embodiment, the invention provides lung specific marker information in computer-accessible form. In other embodiments, methods and compositions of the invention are useful for drug selection, drug evaluation, patient prognosis, and patient monitoring.

Diagnostic methods and arrays of the invention can include all of the markers that are characteristic of one or more classes or subclasses of cancer described herein. Alternatively, single markers can be used. Preferably 1 to 20, 1 to 10, or about 5 genetic markers are used in an assay or on an assay to diagnose or detect a specific type of cancer. A single assay may be used to diagnose or detect one or more classes or subclasses of cancer disclosed herein. A useful assay includes one or more markers of one or more classes or subclasses of cancer. Preferred markers for different classes and subclasses of cancer are shown in Tables 1-9.

Drug screening methods of the invention involve assaying candidate compounds or drugs for their effect on one or more markers of one or more difference classes or subclasses of cancer described herein. Preferably 1 to 20, 1 to 10, or about 5 genetic markers are used in a screening assay to identify a drug that is effective to reduce the expression level of at least one of the markers. Preferred markers for different classes and subclasses of cancer are shown in Tables 1-9. Preferred drug candidates reduce the expression of markers associated with all classes of cancer. However, drug candidates that reduce the expression of markers associated with one or a subset of classes of cancer are also useful. Drug candidates identified in these assays are preferably subject to clinical testing to evaluate their effectiveness against different types of cancer, including different classes and subclasses of lung cancer.

According to the invention, markers shown to be overexpressed in different types of cancer (including different classes or subclasses of lung cancer) can be used as targets for drug development. Useful drugs include antisense nucleic acids that decrease the expression of one or more markets described herein. Useful drugs also include antibodies or other compounds that interfere with the gene product of one or more markers of the invention. For example, a protease inhibitor that inhibits the activity of kallikrein 11 may be therapeutically useful.

### DESCRIPTION OF THE DRAWINGS

FIG. 1. Survival analysis of neuroendocrine C2 adenocarcinomas is shown. Kaplan-Meier curves for C2 versus all other adenocarcinomas. A, All patients. C2 (n=9) and non-C2 (n=117). B, Patients with stage I tumors only. C2 (n=4) and non-C2 (n=72).

FIG. 2. A computer system is shown. The Memory can be a RAM, ROM, CDROM, Tape, Disk, or other form of memory. The Removable data medium can be a magnetic disk, a CDROM, a tape, an optical disk, or other form of removable data medium.

FIG. 3. A box plot of median array intensity across IVT batches is shown and examples of uncorrected and corrected non-linear responses on same specimens following linear and non-linear scaling methods are also shown.

FIG. 4. Non-linear responses in reference RNA samples are shown following linear scaling (a, c and e) that is corrected after rank invariant scaling (b, d and f).

FIG. 5. Pairwise agreement (R.sq values) of 12600 rank invariant scaled expression values of genes are shown between replicate arrays.

FIG. 6. Clusters selected by AutoClass over several runs of the algorithm are shown. The left panel plots the distribution over 200 runs of the algorithm on the original data set (experiment 1), and on the bootstrapped data sets (experiment 2), both defined over 675 genes. The right panel plots the corresponding distributions with respect to the data sets defined over 1514 genes.

### DETAILED DESCRIPTION OF THE INVENTION

The invention provides methods and compositions for classifying lung carcinomas based on gene expression information. In general, the invention relates to the analysis of gene expression information in normal and cancerous lung tissue and the identification of types or classes of lung cancer based on different patterns of gene expression in different lung carcinomas. In addition, the invention provides specific markers of the different types and classes of lung cancer. According to the invention, markers are useful to classify and evaluate new lung cancers, to provide a prognosis for a lung cancer patient, to identify drugs, and to monitor the progression of a lung cancer in a patient.

According to the invention, gene expression can be assayed by analyzing and/or quantifying the nucleic acid (including mRNA, rRNA, tRNA and other RNA products of gene transcription) or protein (including short peptide and other protein translation products) products of gene expression. Methods for measuring gene expression are known in the art, and examples are discussed herein. However, one of ordinary skill in the art will understand that methods of the invention relate to all assays of gene expression in normal or diseased lung samples.

In one embodiment, a gene expression analysis of 186 human carcinomas from the lung provides evidence for biologically distinct sub-classes of lung adenocarcinoma.

More fundamental knowledge of the molecular basis and classification of lung carcinomas is useful in the prediction of patient outcome, the informed selection of currently available therapies, and the identification of novel molecular targets for chemotherapy. The recent development of targeted therapy against the Abl tyrosine kinase for chronic myeloid leukemia illustrates the power of such biological knowledge.

### Molecular Classification of Diverse Lung Tumors

The present invention provides methods for classifying diverse lung tumors based on gene expression profiles. In preferred embodiments, lung tumors are classified based on the expression of a set of marker genes characteristic of a type of lung cancer. In a more preferred embodiment, classification is based on the expression of between 1 and 50, preferably between 1 and 20, more preferably between 1 and 10, and more preferably between 5 and 10 marker genes, the expression of which is strongly correlated with a type of lung cancer.

First, hierarchical clustering (Eisen, M. B., Spellman, P. T., Brown, P. O. & Botstein, D. (1998) Proc Natl Acad Sci USA 95, 14863-8) was applied to classify all 203 samples using the 3312 most variably expressed transcripts. The resulting clusters recapitulated the distinctions between established histologic classes of lung tumors-pulmonary carcinoid tumors, SCLC, squamous cell lung carcinomas, and adenocarcinomasthus validating the experimental and analytic approach of the invention. Two-dimensional hierarchical clustering of 203 lung tumors and normal lung samples was performed with 3,312 transcript sequences. The expression index for each transcript was normalized. Adenocarcinomas resected from the lung and a subset of adenocarcinomas suspected as colon metastases were analyzed.

Normal lung samples form a distinct group, but are most similar to the adenocarcinomas. Marker genes that characterize normal lung samples include TGFβ receptor type II, tetranectin and ficolin 3. A cluster of genes with high relation expression in normal lung includes: TGF-β receptor II; epithelial membrane prot. 2; PECAM-1 (CD31 antigen); PECAM-1 (CD31 antigen); cadherin 5, type 2, VE-cadherin; AF070648; four and a half LIM domains 1; microfibrillar-associated prot. 4; amine oxidase, copper containing 3; A kinase anchor prot. 2; ficolin 3; receptor activity modifying prot. 2; tetranectin; adv. glycosylation end prod.-sp. receptor; TEK tyrosine kinase, endothelial; and slit homolog 2. Elevated TGFβ receptor type II levels have been previously reported for normal bronchial and alveolar epithelium compared to lung carcinomas.

SCLC and carcinoid tumors both show high-level expression of neuroendocrine genes including insulinoma-associated gene 1 (Ball, D. W., Azzoli, C. G., Baylin, S. B., Chi, D., Dou, S., DonisKeller, H., Cumaraswamy, A., Borges, M. & Nelkin, B. D. (1993) Proc Natl Acad Sci USA 90, 5648-52, Lan, M. S., Russell, E. K., Lu, J., Johnson, B. E. & Notkins, A. L. (1993) Cancer Res 53, 4169-71), achaete scute homolog 1 (Ball, D. W., Azzoli, C. G., Baylin, S. B., Chi, D., Dou, S., DonisKeller, H., Cumaraswamy, A., Borges, M. & Nelkin, B. D. (1993) Proc Natl Acad Sci USA 90, 5648-52, Lan, M. S., Russell, E. K., Lu, J., Johnson, B. E. & Notkins, A. L. (1993) Cancer Res 53, 4169-71), gastrin-releasing peptide and chromogranin A. Several previously undescribed markers for SCLC such as thymosin-β and the cell cycle inhibitor p18^{ink4C} were also observed. A cluster of genes with high relative expression in neuroendocrine tumors (small cell lung cancer and pulmonary carcinonas) includes: tubulin, βpolypeptide; insulinoma-associated 1; extra spindle poles, yeast homolog; core-binding factor, (runt), α subunit 2; guanine nucleotide binding prot. 4; achaete-scute homolog-like 1; achaete-scute homolog-like 1; CDKN2C (p18); forkhead box GIB; thymosin p, neuroblastoma; ISL1 transcription factor; distal-less homeobon 6; transcription factor 12 (HTF4); PC4 and SFRS1 interacting prot. 2. In one embodiment of the invention, only a few markers are shared between SCLC and carcinoids, while a distinct group of genes defines carcinoid tumors. Two-dimensional hierarchical clustering of 203 lung tumor and normal samples (data set A) was performed with 3,312 genes as described herein. Different clusters of genes with high relative expressions were observed for normal lung; lung carcinoid; small cell lung carcinoma; squamous cell lung carcinoma; and colon metastasis. Clusters C1, C2, C3 and C4 were defined by clustering of data set B. This suggests that carcinoids are highly divergent from malignant lung tumors.

Squamous cell lung carcinomas, for which diagnostic criteria include evidence of squamous differentiation such as keratin formation form a discrete cluster with high-level expression of transcripts for multiple keratin types and the keratinocytespecific protein stratifin. A cluster of genes with high relative expression in squamous cell lung carcinomas with keratin markers includes: glypican 1; collagen, type VII, α 1; desmoglein 3; W27953; keratin 17; keratin 5; tumor prot. 63; keratin 6; ataxia-telangiectasia group D-assoc. prot.; serine proteinase inhibitor, clade B (5); bullous pemphigoid antigen 1; KIAA0699; CaN19/M87068; S100 calcium-binding prot. A2; and galectin 7. The squamous tumors also show over-expression of p63, a p53-related gene essential for the formation of squamous epithelia. Several adenocarcinomas that express high levels of squamous associated genes, also display histological evidence of squamous features.

Finally, expression of proliferative markers, such as PCNA, thymidylate synthase, MCM2 and MCM6, is highest in SCLC, which is known to be the most rapidly dividing lung tumor A cluster of genes with high relative expression associated with proliferation includes: MCM2; MCM6; Rad2; flap structure-specific endonuclease 1; PCNA; thymidylate synthetase; DEK oncogene; H2A histone family, member Z; high-mobility group prot. 2; and ZW10 interactor. However, unlike the other major lung tumor classes shown above, lung adenocarcinomas were not defined by a unique set of marker genes.

Class Discovery among Lung Adenocarcinomas.

Strong signatures in other lung tumors may obscure the successful subclassification of lung adenocarcinoma in the above analysis. Therefore, a hierarchical clustering was used to sub-classify a data set restricted to adenocarcinomas. Classifications derived by hierarchical clustering and probabilistic clustering algorithms were compared. A two-dimensional colored matrix was generated as a visual representation of a corresponding numerical matrix whose entries record a normalized measure of association strength between samples. Strong association approaches a value of 1 and poor association is close to 0. Associations were obtained for colon metastasis; normal lung; C1 through C4 (adenocarcinoma clusters); additional groups with weaker association were also observed (groups I, II, and III). Genes expressed at high levels in specific subsets of adenocarcinomas can be clustered as a function of histologic differentiation within lung adenoma sub-classes. To avoid spurious variations contributing to the clustering process, 675 transcript sequences were selected with expression levels that were most highly reproducible in duplicate adenocarcinoma samples, yet whose expression varied widely across the chosen sample set (Dataset B); as discussed in the Examples. Normal lung specimens were included in this dataset, as normal epithelium is a component of the grossly dissected adenocarcinoma samples.

To reduce potential classification-bias due to choice of clustering method, and to clarify adenocarcinoma sub-class boundaries, a model-based probabilistic clustering method (Kang, Y., Prentice, M. A., Mariano, J. M., Davarya, S., Linnoila, R. I., Moody, T. W., Wakefield, L. M. & Jakowlew, S. B. (2000) Exp Lung Res 26, 685-707) was also used. To assess the overall strength of each pair-wise association, the frequency with which two samples appeared together was measured in a cluster in 200 clustering iterations over bootstrap data sets. A stable cluster was defined as a set of at least 10 samples with a high degree of association (a threshold of 0.45 was used, corresponding to shared cluster membership in at least 45% of the bootstrap datasets in which both samples were included). According to this definition, several clusters suggested by the hierarchical tree are stable. These associations can be shown, as a color matrix overlaid on a tree structure obtained from hierarchical clustering. The blocks of associated samples show that both clustering methods recognized subclasses corresponding to normal lung and putative colon metastases (CM). Four subclasses of primary lung adenocarcinoma (C 1 to C4) were also observed by both probabilistic and hierarchical clustering. Several smaller and/or less robust groups were also observed (Groups I, II, and III).

Probabilistic clustering also revealed correlations between samples that do not directly cluster together. For example, although cluster C4 falls in the right branch of the hierarchical dendrogram with normal lung, it shows significant association with some subclasses in the left dendrogram (groups I and III and cluster C3) but not with other subclasses (clusters CM, C1, and C2).

Clusters C2, C3, and C4 were also seen as coherent adenocarcinoma groups within the hierarchical clustering of the larger set of lung tumors using the 3,312 transcript sequence set (Dataset A). The reproducible generation of these adenocarcinoma subclasses, across both clustering methods and both gene sets analyzed, supports the validity of the adenocarcinoma clusters and their boundaries.

In order to identify genes that best defined the proposed clusters, a supervised approach was used to extract marker genes from the entire set of 12,600 transcript sequences. For each cluster, selected genes were the most preferentially expressed in the cluster relative to all other samples, using the signal-to-noise metric described previously (Golub, T. R., Slonim, D. K., Tamayo, P., Huard, C., Gaasenbeek, M., Mesirov, J. P., Coller, H., Loh, M. L., Downing, J. R., Caligiuri, M. A., et al. (1999) Science 286, 5317). The genes whose expression correlated best with each class are useful as markers for class prediction of unknown lung cancer samples.

Identification of Adenocarcinomas Metastatic to the Lung.

The present invention provides methods for identifying metastatic tumors of non-lung origin. A key issue in lung tumor diagnosis is the discrimination of a primary lung adenocarcinoma from a distant metastasis to the lung. One distinct hierarchical cluster of 12 samples was identified that most likely represent metastatic adenocarcinomas from the colon. These tumors express high levels of galectin-4, CEACAMI and liverintestinal cadherin 17, as well as c-myc, which is commonly overexpressed in colon carcinoma. Genes expressed at high levels in colon metastases include: c-myc; ETS-2; expressed in thyroid; cadherin 17, (liver-intestine); galectin-4; transmem. 4 superfam. mem. 3; integrin, α 6; trypsin 4, brain; diacylglycerol O-acyltransferase; E74-like factor 3; claudin 4; claudin 3; KIAA0792 gene product; CEA CAM-1; and immediate early response 3. Of the 10 samples in this group for which clinical history and/or histopathologic information was available, only 7 samples had been previously diagnosed as metastases of colonic origin. Other adenocarcinomas that showed nonlung signatures included AD 163, which expressed several breast-associated markers including estrogen receptor and mammaglobin, and was associated with a clinical history and histopathology consistent with breast metastasis. Also, AD368, which was not identified as a metastasis, expressed high levels of albumin, transferrin, and other markers associated with the liver. Thus, clustering identified suspected metastases of extra-pulmonary origin, including some that were previously undetected. Accordingly, methods of the invention can play a pivotal role for gene expression analysis in lung tumor diagnosis.

Molecular Signature of Lung Adenocarcinoma Sub-Classes.

The present invention also provides methods for identifying subclasses of lung adenocarcinoma. Hierarchical and probabilistic clustering defined four distinct sub-classes of primary lung adenocarcinomas. Tumors in the C1 cluster express high levels of genes associated with cell division and proliferation (ubiquitin carrier prot.; Cks-Hs2; high-mobility group prot. 2; flap structure-specific endonuclease 1; MCM6; thymidine kinase 1; PCNA; and W27939), some of which are also expressed in the squamous cell lung carcinoma and SCLC samples in Dataset A. Relatively high-level expression of proliferation-associated genes was also seen in cluster C2.

Several neuroendocrine markers, such as dopa decarboxylase and achaete-scute homolog 1, define cluster C2 (kallikrein 11; dopa decarboxylase; achaete-scute homolog-1; achaete-scute homolog-1; calcitonin-related polypeptide a; proprotein convertase subtilisin; and carboxypeptidase E) and some of these are also expressed in SCLC and pulmonary carcinoids. However, the serine protease, kallikrein 11, is uniquely expressed in the neuroendocrine C2 adenocarcinomas, and not in other neuroendocrine lung tumors.

C3 tumors are defined by high-level expression of two sets of genes. Expression of one gene cluster (ATPase, Na+/K+ transporting; mesothelin; S100 calcium-binding prot. P; solute carrier family 16; KIAA0828; phospholipase A2, group X; progastricsin (pepsinogen C); cytokine receptor-ike factor 1; dual specificity phosphatase 4; ornithine decarboxylase 1; ornithine decarboxylase 1; TS deleted in oral cancer-related 1; ribosomal S6; sodium channel, nonvoltage-gated 1 α; DKFZP56400823; glutathione S-transferase pi; glutathione S-transferase pi; and hepsin), including ornithine decarboxylase 1 and glutathione S-transferase pi, is shared with the neuroendocrine C2 cluster. Expression of the second set of genes is shared with cluster C4 and with normal lung. Genes expressed at high levels in C4, C3 and normal lung include: surfactant, pulmonary-assoc. prot. B; ˜N acylsphingosine amidohydrolase; cytochrome b-5; cytochrome b-5; deleted in liver cancer 1; Ca+ channel, voltage-dependent; surfactant, pulmonary-assoc. prot. C; surfactant, pulmonary-assoc. prot. D; AL049963; ATP-binding cassette (ABC1); KIAA0018 gene product; cathepsin H; selenium binding protein 1; KIAA0758; leukotriene A4 hydrolase; AFO35315; leukocyte protease inhibitor; and BENE. Highest expression of type II alveolar pneumocyte markers, such as thyroid transcription factor 1, and surfactant protein B, C and D genes, was seen in cluster C4, followed by normal lung and C3 cluster. Other markers that defined cluster C4 included cytochrome b5, cathepsin H, and epithelial mucin 1.

Relation Between Gene Expression Tumor Classes, Histological Analysis and Smoking History.

Cluster C1 primarily contains poorly differentiated tumors, while C3 and C4 contains predominantly well-differentiated tumors. Adenocarcinomas of cluster C2 fell in between. Ten of the 14 C4 tumors had been identified as BACs by at least one out of three pathologists who examined the tumors; in contrast, 15 of the remaining 113 adenocarcinomas were similarly described as BACs. The presence of type 11 pneumocyte markers and the high fraction of putative BACs suggest that cluster C4 is likely to be a gene expression counterpart to BAC. All of the C4 tumors in this study were surgical-pathological stage I tumors.

Although microscopic analysis indicated that samples varied in homogeneity, contamination of normal lung cells does not seem to have overwhelmed the expression signatures. The degree to which tumors clustered with normal samples did not reflect the percentage of tumor cells in a sample in most cases. Class C4 is most similar to normal lung in both hierarchical and probabilistic clustering, yet these tumors all revealed at least an estimated 50% tumor nuclei and in most samples over 80%. In contrast, classes C2 and CM contain tumors with as few as 30% estimated tumor nuclei but are sharply distinguishable from the normal lung. Note that only adenocarcinoma specimen AD363, with an estimated 30% tumor content in the adjacent section, clustered with normal lung.

Two adenocarcinoma sub-classes were associated with lower tobacco smoking histories. The presumed metastases of colon origin (CM) and C4 adenocarcinomas with type II pneumocyte gene expression have median smoking histories of 2.5 and 23 pack-years, respectively. The entire data set had a median smoking history of 40 pack-years.

Correlation of Patient Outcome with Putative Adenocarcinoma Classes.

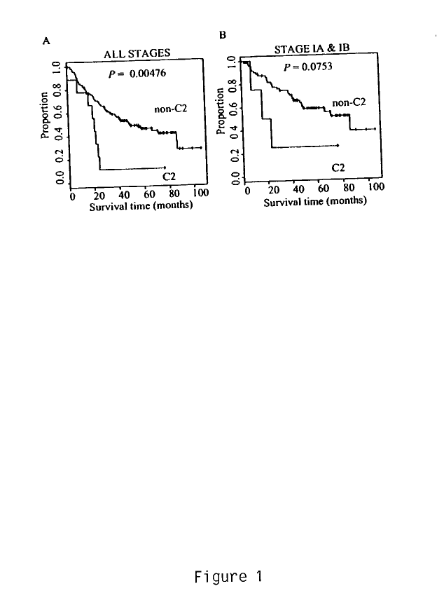

The present invention also provides methods for predicting patient outcome based on the analysis of lung marker gene expression. Lung cancer patient outcome was correlated with the sub-classes of lung adenocarcinomas defined herein. The neuroendocrine C2 adenocarcinomas were associated with a less favorable survival outcome than all other adenocarcinomas (FIGS. 1A, 1B). The median survival for C2 tumors was 21 months compared to 40.5 months for all non-C2 tumors (P=0.00476). When only stage I tumors are considered, the median survival for patients with C2 tumors was 20 months compared to 47.8 months for patients with non-C2 tumors; as the numbers are smaller, the P-value for this comparison is 0.0753. In contrast, C4 adenocarcinomas with type II pneumocyte gene expression (n=14) were associated with a more favorable survival outcome than non-C4 tumors. The median survival for patients with C4 tumors was 49.7 months while the median survival for patients with non-C4 tumors was 33.2 months (P=0.049; note that the non-C2 and non-C4 groups are different because of the exclusion of each group separately in the comparison). For patients with stage I tumors, the median survival in the C4 group was 49.7 months and 43.5 months in the non-C4 group (P=0.191). There was no detectable difference in prognosis between the primary lung adenocarcinomas and the metastases to the lung of colonic origin.

Arrays of Gene Expression Detection Agents.

The present invention also provides arrays of gene expression detection agents. Preferred gene expression detection agents hybridize specifically to marker genes disclosed herein. Such agents may be RNA, DNA, or PNA molecules. Preferred agents are oligonucleotides. Alternative agents bind specifically to the protein expression products of the marker genes disclosed herein. Preferred agents include antibodies and aptamers.

Agents, such as oligonucleotides, are preferably attached to a solid support in the form of an array. Oligonucleotide arrays in the form of gene chips and useful hybridization assays are known in the art and disclosed for example in U.S. Pat. Nos. 5,631,734; 5,874,219; 5,861,242; 5,858,659; 5,856,174; 5,843,655; 5,837,832; 5,834,758; 5,770,722; 5,770,456; 5,733,729; 5,556,752; 6,045,996; and 6,261,776. In a preferred embodiment, an array includes oligonucleotides for measuring the expression level of markers for a specific type or class of lung cancer. In a more preferred embodiment, an array of the invention includes a plurality of oligonucleotides that are specific for marker for several types or classes of lung cancer or adenocarcinoma.

Information about Marker Genes and Marker Gene Expression Levels.

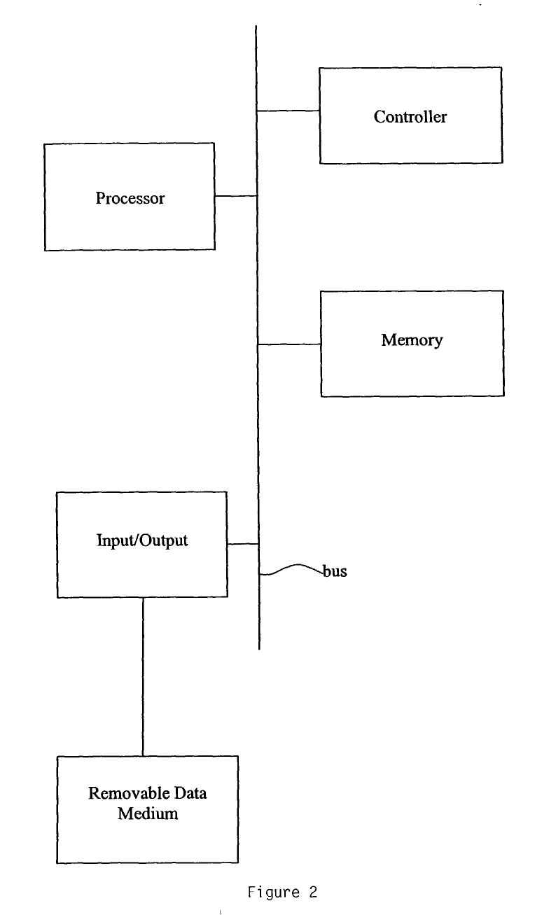

The present invention further provides databases of marker genes and information about the marker genes, including the expression levels that are characteristic of different lung cancer types or lung adenocarcinoma subclasses. According to the invention, marker gene information is preferably stored in a memory in a computer system (FIG. 2). Alternatively, the information is stored in a removable data medium such as a magnetic disk, a CDROM, a tape, or an optical disk. In a further embodiment, the input/output of the computer system can be attached to a network and the information about the marker genes can be transmitted across the network.

Preferred information includes the identity of a predetermined number of marker genes the expression of which correlates with a particular type of lung cancer or a particular subclass of adenocarcinoma. In addition, threshold expression levels of one or more marker genes may be stored in a memory or on a removable data medium. According to the invention, a threshold expression level is a level of expression of the marker gene that is indicative of the presence of a particular type or class of lung cancer.

In a highly preferred embodiment, a computer system or removable data medium includes the identity and expression information about a plurality of marker genes for several types or classes of lung cancer disclosed herein. In addition, information about marker genes for normal lung tissue may be included.

Information stored on a computer system or data medium as described above is useful as a reference for comparison with expression data generated in an assay of lung tissue of unknown disease status.

Finally, the present invention provides methods for identifying, evaluating, and monitoring drug candidates for the treatment of different lung cancer types or adenocarcinoma subclasses. According to the invention, a candidate drug is assayed for its ability to decrease the expression of one or more markers of lung cancer. In one embodiment, a specific drug may reduce the expression of markers for a specific type or subclass of lung carcinoma described herein. Alternatively, a preferred drug may have a general effect on lung cancer and decrease the expression of different markers characteristic of different types or classes of lung carcinoma. In one embodiment, a preferred drug decreases the expression of a lung cancer marker by killing lung cancer cells or by interfering with their replication.

In one embodiment, the screening assays for drug candidates are performed on proteins encoded by the nucleic acids that are identified as having an increased expression in specific subclasses or types of lung carcinoma. In another embodiment, the screening assays for drug candidates are performed on nucleic acids that are differentially expressed in various subclasses or types of lung cancer when compared with normal samples.

In one embodiment, a candidate drug is added to cells or sample tissue prior to analysis. Preferred cells are cell lines grown from different types of cancer (e.g. different classes or subclasses of lung cancer). Alternatively, cells isolated directly from tumor tissue can be assayed. In another embodiment, the invention provides screens for a candidate drug which modulates lung cancer, modulates lung cancer gene expression and/or protein expression, modulates lung cancer genes or protein activity, binds to a lung cancer protein, or interferes with the binding of a lung cancer protein and an antibody.

The term “candidate drug” or equivalent as used herein describes any molecule, e.g., an antibody, protein, oligopeptide, fatty acid, steroid, small organic molecule, polysaccharide, polynucleotide, antisense molecule, ligand, bioactive partner and structural analogs or combinations thereof, to be tested for canditate drugs that are capable of directly or indirectly altering the lung cancer phenotype, or the expression of one or more lung cancer markers as identified herein, or overall gene and/or protein expression. Accordingly, methods of the invention include assays for monitoring the expression of nucleic acids and protein.

Preferred assays screen for candidate drugs that modulate the overall expression of specific gene clusters identified herein (for exampe, one or more genes in Tables 1-9), or the expression of specific nucleic acids or proteins within the clusters. In a particularly preferred embodiment, as assay identified a candidate drug that suppresses a lung cancer phenotype, for example to a normal lung tissue phenotype. A variety of assays can be executed for drug screening. For example, once a specific gene is identified as being differentially expressed by the methods of the invention, candidate drugs that specifically modulate expression or levels of the specific gene may be identified. For example, candidate drugs may be identified that down regulate expression of the specific gene. In one embodiment, candidate drugs may be identified that up regulate expression of the specific gene. Generally a plurality of assay mixtures are run in parallel with different drug concentrations to obtain a differential response to the various concentrations. Typically, one of these concentrations serves as a negative control, i.e., at zero concentration or below the level of detection.

The amount of gene expression can be monitored at either the gene level or the protein level, i.e., the amount of gene expression may be monitored using nucleic acid probes and methods known in the act may be used to qualify gene expression levels. Alternatively, the gene product itself can be monitored, for example through the use of antibodies to the proteins encoded by the nucleic acids identified by the methods of the invention, and in standard immunoassays.

In one embodiment, candidate drugs or agents are naturally occurring proteins or fragments of naturally occurring proteins. Thus, for example, cellular extracts containing proteins, or random or directed digests of proteinaceous cellular extracts, may be used. In this way libraries of prokaryotic and eukaryotic proteins may be made for screening by the methods of the invention. Particularly preferred in this embodiment are libraries of bacterial, fungal, viral, and mammalian proteins, with the latter being preferred, and human proteins being especially preferred.

In another embodiment, candidate drugs are peptides of from about 5 to about 30 amino acids, with from about 5 to about 20 amino acids being preferred, and from about 7 to about 15 being particularly preferred. The peptides may be digests of naturally occurring proteins as is outlined above, random peptides, or “biased” random peptides. By “random” or equivalents herein is meant that each nucleic acid and peptide consists of essentially random nucleotides and amino acids, respectively. Since generally these random peptides (or nucleic acids), are chemically synthesized, they may incorporate any nucleotide or amino acid at any position. The synthetic process can be designed to generate randomized proteins or nucleic acids, to allow the formation of all or most of the possible combinations over the length of the sequence, thus forming a library of randomized candidate proteinaceous drugs.

In another embodiment, the candidate drugs are nucleic acids. As described above generally for proteins, nucleic acid candidate drugs may be naturally occurring nucleic acids or random nucleic acids. For example, digests of prokaryotic or eukaryotic genomes may be used as is outlined above for proteins.

In a preferred embodiment, nucleic acid drug candidates are antisense molecules. Drug candidates that are antisense molecules include antisense or sense oligonucleotides comprising a single-strand nucleic acid sequence (either RNA or DNA) capable of binding to target mRNA or DNA sequences for lung cancer molecules identified by the methods of the invention. For example, a preferred antisense molecule is a molecule that binds a nucleic acid sequence encoding Kallikrein 11. The antisense molecule can either bind a full-length nucleic acid encoding Kallikrein 11, for example the full-length DNA or mRNA encoding Kallikrein 11, or a partial nucleic acid sequence for Kallikrein 11. Antisense or sense oligonuclotides, typically include a fragment of generally about 14 nucleotides, preferably about 14 to 30 nucleotides. However, it is understood that the length of the antisense or sense nucleotides will depend on the length of the target nucleic acid or a fragment thereof.

In yet another preferred embodiment, drug candidates are antibodies. An antibody used in methods for screening for a candidate drug may either bind a full length protein or a fragment thereof. In a preferred embodiment, the antibody binds a unique epitope on a target protein and shows little or no cross-reactivity. The term “antibody” is understood to include antibody fragments, as are known in the art, including Fab, Fab.sub.2, single chain antibodies (Fv for example), chimeric antibodies, etc., either produced by the modification of whole antibodies or those synthesized de novo using recombinant DNA technologies known in the art.

Antibodies as used herein as drug candidates include both polyclonal and monoclonal antibodies. Polyclonal antibodies can be raised in a mammal, for example, by one or more injections of an antigenic agent and, if desired, an adjuvant. It may be useful to conjugate the antigenic agent to a protein known to be immunogenic in the mammal being immunized. Preferred antigenic agents include cancer specific antigens, and more preferably lung cancer specific antigens. Examples of adjuvants which may be employed include Freund's complete adjuvant and MPL-TDM adjuvant (monophosphoryl Lipid A, synthetic trehalose dicorynomycolate).

The antibodies may, alternatively, be monoclonal antibodies. Monoclonal antibodies may be prepared using various hybridoma methods known in the art. For example, a mouse, hamster, or other appropriate host animal, is typically immunized with an immunizing agent to elicit lymphocytes that produce or are capable of producing antibodies that will specifically bind to a immunizing agent. Alternatively, the lymphocytes may be immunized in vitro. An immunizing agent is preferably a protein or fragment thereof that differentially expressed in subclasses or types of lung cancer. However, other known cancer specific antigens may also be used. In a preferred embodiment, the immunizing agent is the full length Kallikrein 11 protein or a homolog or derivative thereof. In another embodiment, the immunizing agent is a partial-length Kallikrein 11 protein or a homolog or derivative thereof.

Panels of available antibodies may also be screened for their effect on the expression of lung specific gene clusters (or specific genes or subsets of genes within these clusters). In one embodiment, some or all o fthe antibodies being screened are not known to be associated with any cancer specific antigen. In one embodiment, the antibodies are bispecific antibodies. Bispecific antibodies are monoclonal, preferably human or humanized, antibodies that have binding specificities for at least two different antigens.

In yet another embodiment, the candidate drugs are chemical compounds. In a preferred embodiment, the candidate drugs are small organic compounds having a molecular weight of more than 100 and less than about 2500 daltons. Candidate drugs may also include functional groups necessary for structural interaction with proteins or nucleic acids.

According to the invention, levels of marker genes disclsosed herein can be used the follow the course of a lung cancer in a patient. Methods of the invention are therefore useful to evalutate the effectiveness of a particular treatment. In addition, methods of the invention are also useful to monitor the progression of a lung cancer in a patient, for example from a C4 to a C3 to a C2 adenocarcinoma.

The identification of candidates that, alone or admixed with other suitable molecules, are competent to treat lung cancer are contemplated by the invention. Further, the production of commercially significant quantities of the aforementioned identified candidates, which are suitable for the prevention and/or treatment of lung, colon, or other cancer is contemplated. Moreover, the invention provides for the production of therapeutic grade commercially significant quantities of therapeutic agents in which any undesirable properties of the initially identified analog, such as in vivo toxicity or a tendency to degrade upon storage, are mitigated.

Methods of preventing and treating cancer, after the identification of an antibody, peptide, peptidomimetic, nucleic acid, or small molecule, include the step of administering a composition including such a compound to a patient.

Nucleic acid molecules (including DNA, RNA, and nucleic acid analogs such as PNA) which are themselves active or which code for active expressed products; peptides; proteins; antibodies; or other chemical compounds isolated and identified, or based upon or derived from ligands isolated and identified according to the invention (also referred to as active compounds or drugs) can be incorporated into pharmaceutical compositions suitable for administration. Such active compounds or drugs include inhibitors identified or constructed as a result of isolating and identifying ligands according to the invention. The drug compounds discovered according to the present invention can be administered to a mammalian host by any route. Thus, as appropriate, administration can be oral or parenteral, including intravenous and intraperitoneal routes of administration. In addition, administration can be by periodic injections of a bolus of the drug, or can be made more continuous by intravenous or intraperitoneal administration from a reservoir which is external (e.g., an i.v. bag). In certain embodiments, the drugs of the instant invention can be therapeutic-grade. That is, certain embodiments comply with standards of purity and quality control required for administration to humans. Veterinary applications are also within the intended meaning as used herein.

The formulations, both for veterinary and for human medical use, of the drugs according to the present invention typically include such drugs in association with a pharmaceutically acceptable carrier therefor and optionally other therapeutic ingredient(s). The carrier(s) can be “acceptable” in the sense of being compatible with the other ingredients of the formulations and not deleterious to the recipient thereof. Pharmaceutically acceptable carriers, in this regard, are intended to include any and all solvents, dispersion media, coatings, antibacterial and antifingal agents, isotonic and absorption delaying agents, and the like, compatible with pharmaceutical administration. The use of such media and agents for pharmaceutically active substances is known in the art. Except insofar as any conventional media or agent is incompatible with the active compound, use thereof in the compositions is contemplated. Supplementary active compounds (identified according to the invention and/or known in the art) also can be incorporated into the compositions. The formulations can conveniently be presented in dosage unit form and can be prepared by any of the methods well known in the art of pharmacy/microbiology. In general, some formulations are prepared by bringing the drug into association with a liquid carrier or a finely divided solid carrier or both, and then, if necessary, shaping the product into the desired formulation.

A pharmaceutical composition of the invention is formulated to be compatible with its intended route of administration. Examples of routes of administration include oral or parenteral, e.g., intravenous, intradermal, inhalation, transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide.

Useful solutions for oral or parenteral administration can be prepared by any of the methods well known in the pharmaceutical art, described, for example, in Remington's Pharmaceutical Sciences, (Gennaro, A., ed.), Mack Pub., 1990. Formulations for parenteral administration also can include glycocholate for buccal administration, methoxysalicylate for rectal administration, or cutric acid for vaginal administration. The parenteral preparation can be enclosed in ampoules, disposable syringes or multiple dose vials made of glass or plastic. Suppositories for rectal administration also can be prepared by mixing the drug with a non-irritating excipient such as cocoa butter, other glycerides, or other compositions that are solid at room temperature and liquid at body temperatures. Formulations also can include, for example, polyalkylene glycols such as polyethylene glycol, oils of vegetable origin, hydrogenated naphthalenes, and the like. Formulations for direct administration can include glycerol and other compositions of high viscosity. Other potentially useful parenteral carriers for these drugs include ethylene-vinyl acetate copolymer particles, osmotic pumps, implantable infusion systems, and liposomes. Formulations for inhalation administration can contain as excipients, for example, lactose, or can be aqueous solutions containing, for example, polyoxyethylene-9-lauryl ether, glycocholate and deoxycholate, or oily solutions for administration in the form of nasal drops, or as a gel to be applied intranasally. Retention enemas also can be used for rectal delivery.

Formulations of the present invention suitable for oral administration can be in the form of discrete units such as capsules, gelatin capsules, sachets, tablets, troches, or lozenges, each containing a predetermined amount of the drug; in the form of a powder or granules; in the form of a solution or a suspension in an aqueous liquid or non-aqueous liquid; or in the form of an oil-in-water emulsion or a water-in-oil emulsion. The drug can also be administered in the form of a bolus, electuary or paste. A tablet can be made by compressing or moulding the drug optionally with one or more accessory ingredients. Compressed tablets can be prepared by compressing, in a suitable machine, the drug in a free-flowing form such as a powder or granules, optionally mixed by a binder, lubricant, inert diluent, surface active or dispersing agent. Moulded tablets can be made by moulding, in a suitable machine, a mixture of the powdered drug and suitable carrier moistened with an inert liquid diluent.

Oral compositions generally include an inert diluent or an edible carrier. For the purpose of oral therapeutic administration, the active compound can be incorporated with excipients. Oral compositions prepared using a fluid carrier for use as a mouthwash include the compound in the fluid carrier and are applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). In all cases, the composition can be sterile and can be fluid to the extent that easy syringability exists. It can be stable under the conditions of manufacture and storage and can be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, methods of preparation include vacuum drying and freeze-drying which yields a powder of the active ingredient plus any additional desired ingredient from a previously sterile-filtered solution thereof.

Formulations suitable for intra-articular administration can be in the form of a sterile aqueous preparation of the drug which can be in microcrystalline form, for example, in the form of an aqueous microcrystalline suspension. Liposomal formulations or biodegradable polymer systems can also be used to present the drug for both intra-articular and ophthalmic administration.

Formulations suitable for topical administration include liquid or semi-liquid preparations such as liniments, lotions, gels, applicants, oil-in-water or water-in-oil emulsions such as creams, ointments or pasts; or solutions or suspensions such as drops. Formulations for topical administration to the skin surface can be prepared by dispersing the drug with a dermatologically acceptable carrier such as a lotion, cream, ointment or soap. In some embodiments, useful are carriers capable of forming a film or layer over the skin to localize application and inhibit removal. Where adhesion to a tissue surface is desired the composition can include the drug dispersed in a fibrinogen-thrombin composition or other bioadhesive. The drug then can be painted, sprayed or otherwise applied to the desired tissue surface. For topical administration to internal tissue surfaces, the agent can be dispersed in a liquid tissue adhesive or other substance known to enhance adsorption to a tissue surface. For example, hydroxypropylcellulose or fibrinogen/thrombin solutions can be used to advantage. Alternatively, tissue-coating solutions, such as pectin-containing formulations can be used.

For inhalation treatments, inhalation of powder (self-propelling or spray formulations) dispensed with a spray can, a nebulizer, or an atomizer can be used. Such formulations can be in the form of a finely comminuted powder for pulmonary administration from a powder inhalation device or self-propelling powder-dispensing formulations. In the case of self-propelling solution and spray formulations, the effect can be achieved either by choice of a valve having the desired spray characteristics (i.e., being capable of producing a spray having the desired particle size) or by incorporating the active ingredient as a suspended powder in controlled particle size. For administration by inhalation, the compounds also can be delivered in the form of an aerosol spray from a pressured container or dispenser which contains a suitable propellant, e.g., a gas such as carbon dioxide, or a nebulizer. Nasal drops also can be used.

Systemic administration also can be by transmucosal or transdermal means. For transmucosal or transdermal administration, penetrants appropriate to the barrier to be permeated are used in the formulation. Such penetrants generally are known in the art, and include, for example, for transmucosal administration, detergents, bile salts, and filsidic acid derivatives. Transmucosal administration can be accomplished through the use of nasal sprays or suppositories. For transdermal administration, the active compounds typically are formulated into ointments, salves, gels, or creams as generally known in the art.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials also can be obtained commercially from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811. Microsomes and microparticles also can be used.

Oral or parenteral compositions can be formulated in dosage unit form for ease of administration and uniformity of dosage. Dosage unit form refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Generally, the drugs identified according to the invention can be formulated for parenteral or oral administration to humans or other mammals, for example, in therapeutically effective amounts, e.g., amounts which provide appropriate concentrations of the drug to target tissue for a time sufficient to induce the desired effect. Additionally, the drugs of the present invention can be administered alone or in combination with other molecules known to have a beneficial effect on the particular disease or indication of interest. By way of example only, useful cofactors include symptom-alleviating cofactors, including antiseptics, antibiotics, antiviral and antifungal agents and analgesics and anesthetics.

Where a peptide, peptidomimetic, small molecule or other drug identified according to the invention is to be used as part of a transplant procedure (e.g. a lung transplant procedure), it can be provided to the living tissue or organ to be transplanted prior to removal of tissue or organ from the donor. The drug can be provided to the donor host.

Alternatively, or in addition, once removed from the donor, the organ or living tissue can be placed in a preservation solution containing the drug. In all cases, the drug can be administered directly to the desired tissue, as by injection to the tissue, or it can be provided systemically, either by oral or parenteral administration, using any of the methods and formulations described herein and/or known in the art.

Where the drug comprises part of a tissue or organ preservation solution, any commercially available preservation solution can be used to advantage. For example, useful solutions known in the art include Collins solution, Wisconsin solution, Belzer solution, Eurocollins solution and lactated Ringer's solution. Generally, an organ preservation solution usually possesses one or more of the following properties: (a) an osmotic pressure substantially equal to that of the inside of a mammalian cell (solutions typically are hyperosmolar and have K+ and/or Mg++ ions present in an amount sufficient to produce an osmotic pressure slightly higher than the inside of a mammalian cell); (b) the solution typically is capable of maintaining substantially normal ATP levels in the cells; and (c) the solution usually allows optimum maintenance of glucose metabolism in the cells. Organ preservation solutions also can contain anticoagulants, energy sources such as glucose, fructose and other sugars, metabolites, heavy metal chelators, glycerol and other materials of high viscosity to enhance survival at low temperatures, free oxygen radical inhibiting and/or scavenging agents and a pH indicator. A detailed description of preservation solutions and useful components can be found, for example, in U.S. Pat. No. 5,002,965, the disclosure of which is incorporated herein by reference.

The effective concentration of the drugs identified according to the invention that is to be delivered in a therapeutic composition will vary depending upon a number of factors, including the final desired dosage of the drug to be administered and the route of administration. The preferred dosage to be administered also is likely to depend on such variables as the type and extent of disease or indication to be treated, the overall health status of the particular patient, the relative biological efficacy of the drug delivered, the formulation of the drug, the presence and types of excipients in the formulation, and the route of administration. In some embodiments, the drugs of this invention can be provided to an individual using typical dose units deduced from the earlier-described mammalian studies using non-human primates and rodents. As described above, a dosage unit refers to a unitary, i.e. a single dose which is capable of being administered to a patient, and which can be readily handled and packed, remaining as a physically and biologically stable unit dose comprising either the drug as such or a mixture of it with solid or liquid pharmaceutical diluents or carriers.

In certain embodiments, organisms are engineered to produce drugs identified according to the invention. These organisms can release the drug for harvesting or can be introduced directly to a patient. In another series of embodiments, cells can be utilized to serve as a carrier of the drugs identified according to the invention.

The pharmaceutical compositions can be included in a container, pack, or dispenser together with instructions for administration.

Drugs identified by a method of the invention also include the prodrug derivatives of the compounds. The term prodrug refers to a pharmacologically inactive (or partially inactive) derivative of a parent drug molecule that requires biotransformation, either spontaneous or enzymatic, within the organism to release the active drug. Prodrugs are variations or derivatives of the compounds of the invention which have groups cleavable under metabolic conditions. Prodrugs become the compounds of the invention which are pharmaceutically active in vivo, when they undergo solvolysis under physiological conditions or undergo enzymatic degradation. Prodrug compounds of this invention can be called single, double, triple, and so on, depending on the number of biotransformation steps required to release the active drug within the organism, and indicating the number of functionalities present in a precursor-type form. Prodrug forms often offer advantages of solubility, tissue compatibility, or delayed release in the mammalian organism (see, Bundgard, Design of Prodrugs, pp. 7-9, 21-24, Elsevier, Amsterdam 1985 and Silverman, The Organic Chemistry of Drug Design and Drug Action, pp. 352-401, Academic Press, San Diego, Calif., 1992). Prodrugs commonly known in the art include acid derivatives known to practitioners of the art, such as, for example, esters prepared by reaction of the parent acids with a suitable alcohol, or amides prepared by reaction of the parent acid compound with an amine, or basic groups reacted to form an acylated base derivative. Moreover, the prodrug derivatives of drugs discovered according to this invention can be combined with other features herein taught to enhance bioavailability.

Drugs as identified by the methods described herein can be administered to individuals to treat (prophylactically or therapeutically) various stages or subclasses of cancer. In conjunction with such treatment, pharmacogenomics (i.e., the study of the relationship between an individual's genotype and that individual's response to a foreign compound or drug) can be considered. Differences in metabolism of therapeutics can lead to severe toxicity or therapeutic failure by altering the relation between dose and blood concentration of the pharmacologically active drug. Thus, a physician or clinician can consider applying knowledge obtained in relevant pharmacogenomics studies in determining whether to administer a drug as well as tailoring the dosage and/or therapeutic regimen of treatment with the drug.

Pharmacogenomics deals with clinically significant hereditary variations in the response to drugs due to altered drug disposition and abnormal action in affected persons. See e.g., Eichelbaum, M., Clin Exp Pharmacol Physiol, 1996, 23(10-11):983-985 and Linder, M. W., Clin Chem, 1997, 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitroflirans) and consumption of fava beans.

One pharmacogenomics approach to identifying genes that predict drug response, known as “a genome-wide association,” utilizes a high-resolution map of the human genome consisting of already known gene-related markers (e.g., a “bi-allelic” gene marker map which consists of 60,000-100,000 polymorphic or variable sites on the human genome, each of which has two variants). Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. A SNP is a common alteration that occurs in a single nucleotide base in a stretch of DNA. For example, a SNP can occur once per every 1000 bases of DNA. A SNP can be involved in a disease process, however, the vast majority can not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that can be common among such genetically similar individuals.

Alternatively, a method termed the “candidate gene approach,” can be utilized to identify genes that predict drug response. According to this method, if a gene that encodes a drug's target is known, all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification. Alternatively, a method termed the “gene expression profiling,” can be utilized to identify genes that predict drug response. For example, the gene expression of an animal dosed with a drug can give an indication whether gene pathways related to toxicity have been turned on.

Information generated from more than one of the above pharmacogenomics approaches can be used to determine appropriate dosage and treatment regimens for prophylactic or therapeutic treatment an individual. This knowledge, when applied to dosing or drug selection, can avoid adverse reactions or therapeutic failure and thus enhance therapeutic or prophylactic efficiency when treating a subject with a drug identified according to the invention.

### EXAMPLES

### Example 1

### Materials and Methods

Specimens and Datasets.

A total of 203 snap-frozen lung tumors (n=186) and normal lung (n=17) specimens were used to create two datasets. Of these, 125 adenocarcinoma samples were associated with clinical data and with histological slides from adjacent sections.

The 203 specimens (Dataset A) include histologically-defined lung adenocarcinomas (n=127), squamous cell lung carcinomas (n=21), pulmonary carcinoids (n=20), SCLC (n=6) cases and normal lung (n=17) specimens. Other adenocarcinomas (n=12) were suspected to be extrapulmonary metastases based on clinical history. Dataset B, a subset of Dataset A, includes only adenocarcinomas and normal lung samples.

Tumor Bank, Clinical Information, and Pathological Analysis

The complete cohort for these studies consists of 203 patient samples that can be broken down into 139 lung adenocarcinomas (AD) that included 12 suspected metastases of extrapulmonary origin, 21 squamous (SQ) cell carcinoma cases, 20 pulmonary carcinoid (COID) tumors and 6 small cell lung cancers (SCLC), as well as 17 normal lung (NL) samples.

Tumor and normal lung specimens in this study were obtained from two independent tumor banks. The following specimens were obtained from the Thoracic Oncology Tumor Bank at the Brigham and Women's Hospital/Dana Farber Cancer Institute: 127 adenocarcinomas, 8 squamous cell carcinomas, 4 small cell carcinomas, and 14 pulmonary carcinoid samples. In addition 12 adenocarcinoma samples without associated clinical data were obtained from the Brigham/Dana-Farber tumor bank. In addition, 13 squamous cell carcinoma, 2 small cell lung carcinoma, and 6 carcinoid samples were obtained from the Massachusetts General Hospital (MGH) Tumor Bank. The snap-frozen, anonymized samples from MGH were not associated with histological sections or clinical data.

Frozen samples of resected lung tumors and parallel “normal” (grossly uninvolved) lung (protocol 91-03831) for anonymous distribution to IRB-approved research projects were obtained within 30 minutes of resection and subdivided into samples (˜100 mg). Samples intended for nucleic acid extraction was snap frozen on powdered dry ice and individually stored at −140° C. Each was associated with an immediately adjacent sample embedded for histology in Optimal Cutting Temperature (OCT) medium and stored at −80° C. Six micron frozen sections of embedded samples stained with H&E was used to confirm the post operative-pathologic diagnosis and to estimate the cellular composition of adjacent extraction samples as discussed below. Each selected sample was further characterized by examining viable tumor cells in H&E stained frozen sections comprising of at least 30% nucleated cells and low levels of tumor necrosis (<40%). In addition, at least once pulmonary pathologists (I and II) independently evaluated adjacent OCT blocks for tumor type and content. Notes were also taken for extent of fibrosis and inflammatory infiltrates.

Duplicate blocks, coupled with the identical OCT-embedded block, were also available for 36 of the adenocarcinoma samples. The majority of these duplicate blocks were within 1 to 1.5 cm from one another.

Clinical data from a prospective database and from the hospital records included the age and sex of the patient, smoking history, type of resection, post-operative pathological staging, post-operative histopathological diagnosis, patient survival information, time of last follow-up interval or time of death from the date of resection, disease status at last follow-up or death (when known), and site of disease recurrence (when known). Code numbers were assigned to samples and correlated clinical data. The linkup between the code numbers and all patient identifiers was destroyed, rendering the samples and clinical data completely anonymous.

125 adenocarcinoma samples were associated with clinical data. Adenocarcinoma patients included 53 males and 72 females. There were 17 reported non-smokers, 51 patients reporting less than a 40 pack-year smoking history, and 54 patients reported a greater than 40 pack-year smoking history. The post-operative surgical-pathological staging of these samples included 76 stage I tumors, 24 stage II tumors, 10 stage III tumors, and 12 patients with putative metastatic tumors. Note that numbers do not always add to 125, as complete information could not be found for each case.

RNA extraction and Microarray Experiments

Briefly, tissue samples were homogenized in Trizol (Life Technologies, Gaithersburg, Md.) and RNA was extracted and purified using the RNEASY column purification kit (QIAGEN, Chatsworth, Calif.). RNA extracted from samples that were collected from two different OCT blocks was given the sample code name followed by the corresponding OCT block name. Denaturing formaldehyde gel electrophoresis followed by northern blotting using a beta-actin probe assessed RNA integrity. Samples were excluded if beta-actin was not full-length.

Preparation of in vitro transcription (IVT) products and oligonucleotide array hybridization and scanning were performed according to Affymetrix protocol (Santa Clara, Calif.). In brief, the amount of starting total RNA for each IVT reaction varied between 15 and 20 mg. First strand cDNA synthesis was generated using a T7-linked oligo-dT primer, followed by second strand synthesis. IVT reactions were performed in batches to generate cRNA targets containing biotinylated UTP and CTP, which was subsequently chemically fragmented at 95° C. for 35 minutes. Ten micrograms of the fragmented, biotinylated cRNA was mixed with MES buffer (2-[N-Morpholino]ethansulfonic acid) containing 0.5 mg/ml acetylated bovine serum albumin (Sigma, St. Louis, Mo.) and hybridized to Affymetrix (Santa Clara, Calif.) HGU95A v2 arrays at 45° C. for 16 hours. HGU95A v2 arrays contain ˜12600 genes and expressed sequence tags. Arrays were washed and stained with streptavidin-phycoerythrin (SAPE, Molecular Probes). Signal amplification was performed using a biotinylated anti-streptavidin antibody (Vector Laboratories, Burlingame, Calif.) at 3 μg/ml. A second staining with SAPE followed this. Normal goat IgG (2 mg/ml) was used as a blocking agent. Scans on arrays were performed on Affymetrix scanners and the expression value for each gene was calculated using Affymetrix GENECHIP software. Minor differences in microarray intensity were corrected using a scaling method as detailed below.

### Example 2

### Data Analysis

Feature Selection and Hierarchical Clustering.

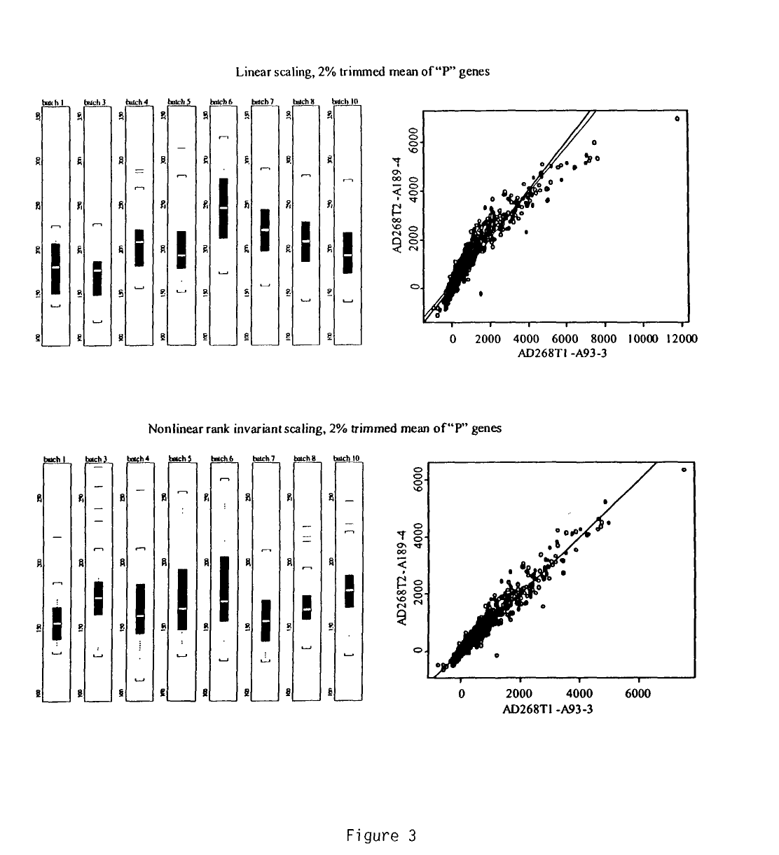

For Dataset A, a standard deviation threshold of 50 expression units was used to select the 3,312 most variable transcript sequences. For Dataset B, 52 pairs of replicates (representing 36 duplicate adenocarcinomas) were used to determine the quality of the dataset, and 45 pairs having a R² value >0.9 were used to select 675 transcript sequences (features) whose expression varied the most across all sample pairs (FIGS. 3-5).

Preprocessing and Re-scaling

The raw expression data for the first 12600 genes obtained from Affymetrix GENECHIP software was re-scaled to account for different chip intensities. Each column (sample) in the dataset was multiplied by 1/slope of a least squares linear fit of the sample vs. the reference (a sample in the dataset). The linear fit was done using only genes that have ‘Present’ calls in both the sample being re-scaled and the reference. The sample chosen as reference was a typical one (i.e. one with the number of “P” calls closer to the average over all samples in the dataset). The reference sample for the dataset was AD114T1. Scans were rejected if the scaling factor exceeded a factor of 4, fewer than 30% ‘Present’ calls, or microarray artifacts were visible. Scans that failed the above criterion were re-hybridized and re-scanned on new chips from the same fragmented cDNA.

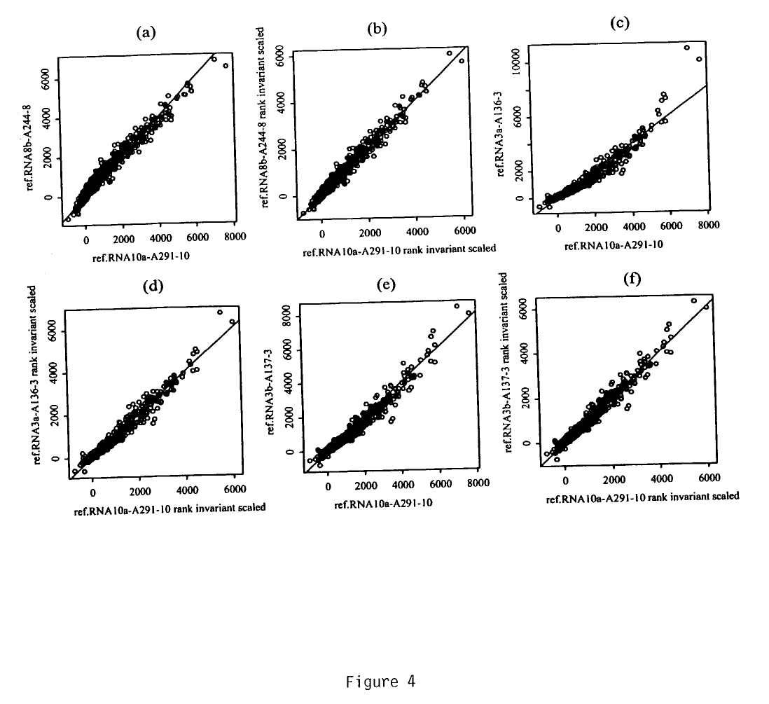

However, linear scaling was insufficient to correct for non-linear responses that were observed, which may have resulted from saturation effects or IVT-variations from one batch to the other. Thus, a non-linear scaling was applied to adjust for such differences (FIG. 3). The 2% trimmed mean of “P” genes for all arrays after linear and non-linear rank invariant scaling (described below) are shown in box plots stratified by IVT batches. The batch differences in mean intensity may be due to the fact that a more homogenous IVT processing was applied to arrays in the same IVT batch than arrays in different batches. Also noticeable was the non-linear relationships between the scatter-plots of replicate arrays (FIG. 3) and reference RNA samples (FIG. 4), which justifies non-linear scaling methods to make expression values of genes across arrays more reasonable estimates of the actual expression values for transcripts and overall brightness of arrays.

A rank-invariant scaling method (Tseng, G. C., Oh, M. K., Rohlin, L., Liao, J. C. & Wong, W. H. (2001) Nucleic Acids Res 29, 2549-57) was used to scale all arrays towards a baseline array (AD114T1). A set of genes whose ranks in the two arrays was smaller than 50 (an empirical value chosen to make the points for selected genes naturally form a tight curve, was used to fit a smoothing spline (Venables, W. N. & Ripley, B. D. (1998) Modern applied statistics with S-PLUS (Springer, Berlin)) in the scatter-plot of the array to be normalized (X-axis) and the baseline array (Y-axis). This “Invariant Set” presumably consists of non-differentially expressed genes. The normalized values were determined by reading off the values determined by the smoothing curve for values on X-axis. After scaling the replicate arrays agree better, and batch differences were less dramatic (FIG. 3). Hence, the rank invariant-scaled data was used for all downstream analysis.

Reproducibility Statistics

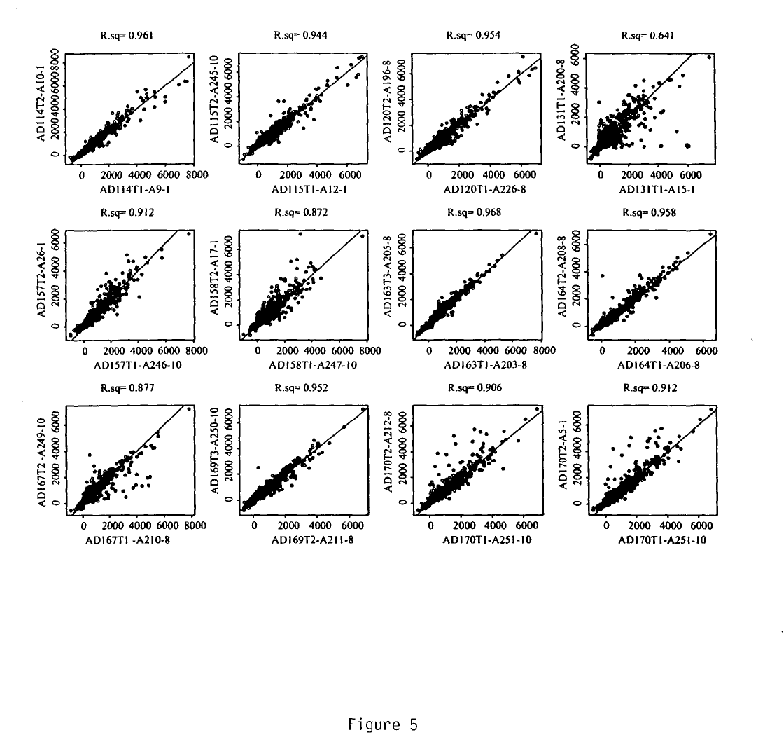

Reproducibility controls included independent frozen tissue blocks for 36 adenocarcinomas resected from the lung, 16 replicates of IVT reactions or scans, and 13 reference RNA samples (Stratagene, La Jolla, Calif.). Scaled expression values for 45 of the 52 replicates compared were correlated with R²>0.9, and for 50 of the 52 replicates with R²>0.85. Examples of pairwise correlations between replicates are shown in FIG. 5.

Replication Filtering

According to the invention, technical noise may affect the measurement of some genes more than others, and the already difficult problem of adenocarcinoma sub-classification might be particularly sensitive to such noise. Accordingly, adenocarcinoma replicates were used to select only highly reproducible features (representing genes) for subsequent use in adenocarcinoma clustering. The reproducibility of 52 pairs of replicate arrays randomly selected across the adenocarcinoma samples was assessed. For each pair of replicates, a single measure of correlation (R²) was computed across all 12600 genes (FIG. 5). Forty-five replicate pairs with R² values greater than 0.9 were used for filtering genes (below).

For each gene, a scatter plot was generated with the selected 45 pairs of replicate data points. The reproducibility of expression was assessed (Pearson correlation) between replicate pairs as well as the variability of expression values across the 45 pairs. The distribution of 45 pairwise expression datapoints was plotted for genes that were randomly selected. The correlation index of expression (a measure of a gene's variability between samples). To avoid spurious correlation measures 2-4 outliers in each dimension were removed from the calculation of correlation was obtained (cluster Incl W26626:, cor=0.0221; desmoglein 3 (pemphi, cor=0.354; phosphoglucomutase 5, cor=0.311; ATP synthase, H+tra, cor=0.137;Cluster Incl A14316, cor=0.188; Cluster Incl Y12851, cor=0.2631, solute carrier famil, cor=0.429; zinc finger protein, cor=0.179; Cluster Incl AA5866, cor=0.374; Cluster Incl AA5866, cor=0.315; Cluster Incl M34428, cor=0.351; ets variant gene 2, cor=0.187; RecQ protein-like 5, cor=0.366; Cluster Incl AJ0100, cor=0.378; one cut domain, fami, cor=0.396; hexose-6-phosphate d, cor=0.0165; Cluster Incl AL0223, cor=0.376; synovial sarcoma, X, cor=0.371; Cluster Incl S79325, cor=0.502; Cluster Incl Z84717: and cor=0.513). In addition, genes whose expression levels did not vary significantly across the 45 samples were eliminated because they were unlikely to be informative. The number of features (genes) selected by this filter varied depending on the Pearson correlation cut-off used. A clustering of adenocarcinomas was performed using 675 genes selected by a Pearson correlation threshold of 0.8. These genes have consistent expression values between replicate arrays, and their expression across all adenocarcinoma samples was variable. Selection of genes at Pearson correlation coefficients of 0.7 (1514 genes), 0.75 (1105 genes), or 0.85 (366 genes) led to roughly similar clustering. The distribution of 45 pairwise expression datapoints was plotted for selected genes that varied between the 45 adenocarcinoma replicates. The spread of the datapoints results in a correlation index that can be used to select genes that are variant between adenocarcinomas. Gene sets were selected based on their correlation cutoffs (0.7, 0.75, 0.8 and 0.85). To avoid spurious correlation measure 2-4 outliers in each dimension were removed from the calculation of correlation. The expression ranges of genes in samples that pass a replicate correlation greater than 0.85 include glyceraldehyde-3-pho, cor=0.873; glycetaldehyde-3-pho, cor=0.861; trefoil factor 3, cor=0.966; thymosin, beta 10, cor=0.862; ribosomal protein L8, cor=0.867; immunoglobulin kappa, cor=0.854; ribosomal protein S1, cor=0.882; melanoma antigen, fa, cor=0.85; epithelial protein u, cor=0.889; metallothionein IF (cor=0.88; surfactant, pulmonar, cor=0.921; UDP glycosyltransfer, cor=0.931; melanoma antigen, fa, cor=0.938; phospholipase A2, gr, cor=0.888; proline oxidase homo, cor=0.871; melanoma antigen, fa, cor=0.922; ring finger protein, cor 0.91; Cluster Incl AF0151, cor 855; tubulin, alpha, ubiq, cor=0.851, and secretory leukocyte, cor=0.934.

Hierarchical Clustering

Hierarchical clustering is an unsupervised learming method useful for dividing data into natural groups. Data are clustered hierarchically by organizing the data into a tree structure based upon the degree of correlation between features. CLUSTER (Eisen, M. B., Spellman, P. T., Brown, P. O. & Botstein, D. (1998) Proc Natl Acad Sci USA 95, 14863-8) was used to perform average linkage clustering of both genes and arrays, using median centering and normalization, and the results were displayed using TREEVIEW (Eisen, M. B., Spellman, P. T., Brown, P. O. & Botstein, D. (1998) Proc Natl Acad Sci USA 95, 14863-8). This organizes all of the data elements into a single tree with the higher levels of the tree representing the discovered classes. A threshold of 0 units was imposed before clustering because the negative values may contribute to artifacts. After this preprocessing, a set of genes was selected for clustering. For Dataset A, a variation filter was used that required a standard deviation greater than or equal to 50 expression units across samples, and 3,312 genes were selected. More stringent variation filters were selected (as few as 900 genes), which produced similar clustering results. For dataset B, 675 genes were selected based on the replicate filtering described above.

In summary, a hierarchical clustering was performed on two data sets: Dataset A, with 203 samples, and a subset, Dataset B, with 156 samples. Two distinct gene selections were used (3,312 genes selected by standard deviation in FIG. 1 versus 675 genes selected by replication filtering. To compare the results of these analyses, the clusters defined in the adenocarcinomas were mapped onto a tree generated using 3,312 genes. Clusters C2, C3 and C4 of the adenocarcinomas form consistently in both analyses.

Probabilistic Clustering

In order to validate the taxonomy obtained by hierarchical clustering, a model-based probabilistic clustering was also used (Cheeseman, P. & Stutz, J. (1996) in Advances in Knowledge Discovery and Data Mining, eds. Fayyad, U. M., Piatetsky-Shapiro, G., Smyth, P. & Uthurasamy, R. (MIT Press, Cambridge), Titterington, D. M., Smith, A. F. & Makov, U. F. (1985) Statistical Analysis of Finite Mixture Distributions (John Wiley, New York)), and the number and composition of clusters obtained by the two methods were compared. The specific program used for probabilistic clustering is AutoClass (Cheeseman, P. & Stutz, J. (1996) in Advances in Knowledge Discovery and Data Mining, eds. Fayyad, U. M., Piatetsky-Shapiro, G., Smyth, P. & Uthurasamy, R. (MIT Press, Cambridge). The method allows for the automatic selection of the number of clusters, and it performs a soft partitioning of the data, whereby each sample can be fractionally assigned to more than one cluster, thus reflecting the inherent uncertainty in the data (in practice, in all experiments samples were assigned to a cluster with probability 1). Probabilistic model-based clustering, usually referred to as finite-mixture models (Titterington, D. M., Smith, A. F. & Makov, U. F. (1985) Statistical Analysis of Finite Mixture Distributions (John Wiley, New York)), is built on the assumption that the observed data can be partitioned into sub-populations (clusters), each governed by a distinct probability distribution. Since a priori the cluster membership is not known, the resulting distribution of the observed data is a mixture of the sub-population distributions. Learning, or inducing, the probabilistic model generating the observed data thus entails determining the number of clusters (model selection), as well as the parameters of the sub-population distributions (parameter estimation). The model selection is based on a Bayesian score that measures the posterior probability of the model given the observed data. Assuming all models are a priori equally likely, this translates into searching for the model that assigns the highest probability to the observed data (i.e which best “explains” the data). It should be emphasized that the Bayesian score incorporates a component that penalizes model complexity (the higher the number of clusters, the higher the complexity of the model), thus automatically controlling for over-fitting. The parameter estimation for this type of modelling is a combinatorial optimization problem for which an exact solution is computationally infeasible. Therefore, an approximate solution needs to be adopted. AutoClass adopts the Expectation-Maximization algorithm (EM), an iterative procedure that, starting from a random initialization of the parameters, incrementally adjusts them in an attempt to find their maximum likelihood estimates (under rather general conditions, the procedure is guaranteed to converge to a local maximum) (Dempster, A. P., Laird, N. M. & Rubin, D. B. (1977) J Royal Stat Soc 39, 398-409, McLachlan, G. J. & Krishnan, T. (1997) The EM Algorithm and Extensions (John Wiley, New York). It is important to point out that because of this random component in the estimation procedure, different runs of the learning algorithms may yield different results (i.e., different parameters—and consequently, different numbers of clusters—may be selected), a variability that is accounted for in the experimental evaluation.

Experimental Evaluation of Probabilistic Clustering

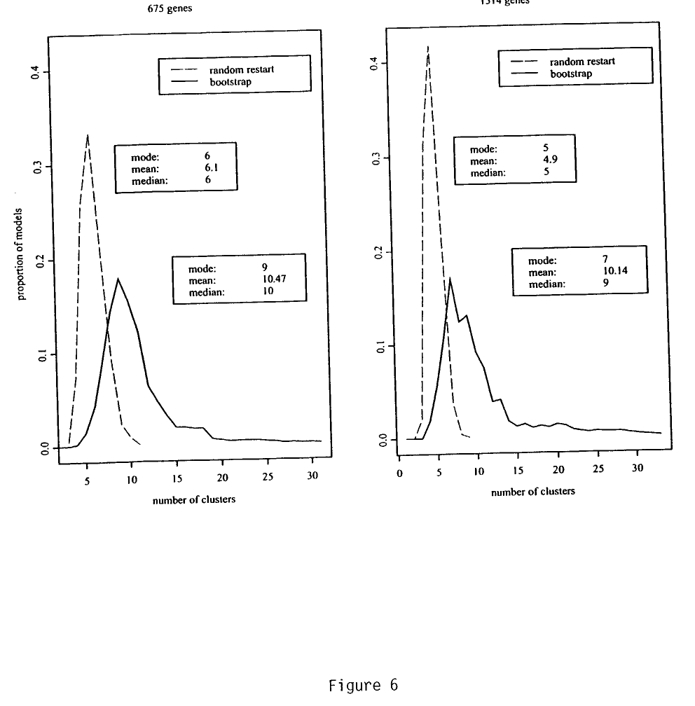

A model-based probabilistic clustering was applied to a data set of 156 samples (Dataset B). For the selection of the genes, the replicate filtering method was used as described above. Two feature sets were used, the first including 675 genes (obtained by setting the correlation threshold at 0.8), and the second including 1514 genes (correlation threshold setting of 0.7). The use of different feature sets was aimed at testing for the sensitivity of the clustering procedure to the number of genes included. AutoClass was then applied to the resulting data set. For each feature set, two sets of experiments were run. In the first experiment (Experiment 1), the learning algorithms were run 200 times, with the only difference between successive runs being in the random initialization of the model parameters. The aim of this experiment was to try to account for variability due to the approximate nature of the estimation procedure. In the second experiment (Experiment 2), the learning algorithms were run 200 times on “bootstrapped” data sets, where a bootstrapped data set was obtained by randomly picking, with replacement, 156 samples from the original data set. The bootstrapped data set differs from the original one in that some of the samples may appear in it multiple times, while other samples may be missing altogether. This experiment was aimed at testing for the robustness of the clustering results to random variations in the observed data. FIG. 6 shows the distribution of the number of clusters over multiple runs for the different settings. As expected, the variability in the number of clusters over multiple iterations was higher in Experiment 2 (bootstrapping) than in Experiment 1 (random restart). This was due to the fact that in a bootstrapped data set, it often happens that the same sample is included more than once (on average, over 200 iterations, each bootstrap data set contained about 100 of the 156 samples in the original data set. In other words, on average 56 samples were duplications of samples already included). If a sample was included a sufficient number of times, the clustering algorithm may find it appropriate to define a cluster for that sample only, thus artificially inflating the number of clusters. Despite this variability, it was reassuring to see that this alternative clustering methodology selected a number of clusters mostly varying between 6 and 9, very close to the number of clusters selected by hierarchical clustering.

A visualization method was used to control for the consistency of the cluster composition over multiple runs, as well as to compare the clusters found by AutoClass with the ones obtained by hierarchical clustering. A colored matrix that is a color-based rendition of a corresponding symmetric matrix whose entries record a normalized measure of how often two samples appear in the same cluster across multiple runs. Rows and columns in this matrix were indexed by the samples in the data set, thus yielding a 156×156 matrix, with each entry taking a real value between 0 and 1. An entry set to 0 (1) indicates that the two samples indexing that entry never (always) appear in the same cluster. More specifically, given two samples, the corresponding entry in the matrix records the quantity N_{match}/Nₜₒₜₐₗ, where Nₜₒₜₐₗ is the number of iterations in which both samples are included, and N_{match} denotes the number of iterations in which the two samples are included and are clustered together. That Nₜₒₜₐₗ is equal to the total number of iterations in Experiment 1, but not in Experiment 2, where it can often happen that a sample is not selected at all in a given iteration.

Ideally, all entries in the matrix are either 0 or 1, corresponding to the situation where the cluster composition remains unchanged over multiple runs of the algorithm. Furthermore, if the samples are arranged in the matrix in the order produced by hierarchical clustering, a perfect agreement between the two clustering methodologies would translate into a block-diagonal matrix with blocks of 1's along the diagonal—each block corresponding to a different cluster—surrounded by 0's. Two-dimensional matrices were generated corresponding, respectively, to Experiment 1 (200 iterations with random restart on the original data set) and Experiment 2 (200 iterations on bootstrap data sets) for the 675-gene data set. Corresponding two-dimensional matrices were generated for the 1514-gene data set. Blocks corresponding to the candidate clusters are clearly distinguishable along the diagonal in all four of the two-dimensional matrices, thus providing supporting evidence that the selected clusters were unaffected by random variations in the data set.

K-Nearest Neighbor-based Marker Gene Selection and Supervised Learning

Following definition of “classes” and their boundaries, a k-NN algorithm was used to choose “marker” genes whose expression best correlated with each class distinction. Class definitions were based on clustering. Marker genes were chosen based on the signal-to-noise statistic (M_{class0}−M_{class1})/(_{class0}+_{class1}), where M and represent the mean and standard deviation of expression, respectively, for each class (Golub, T. R., Slonim, D. K., Tamayo, P., Huard, C., Gaasenbeek, M., Mesirov, J. P., Coller, H., Loh, M. L., Downing, J. R., Caligiuri, M. A., et al. (1999) Science 286, 531-7).

As a further test of the relative robustness of the sample clusters, a supervised classifier was built using the following methodology. Following marker gene selection, a classifier was built and evaluated through leave-one-out cross-validation. For each round of cross-validation, one sample was withheld and the remaining samples were used to build a “k-NN” classifier (see below), from which class membership of the withheld sample was predicted. The top 25 genes selected by signal-to-noise metric for each class are shown in Table 9.

A weighted implementation of the k-NN algorithm that predicts the class of a new sample by selecting the calculating the Euclidean distance (d) of this sample to the k “nearest neighbor” samples in “expression” space in the training set was used, and the predicted class was selected to be that of the majority of the k samples (Dasarathy, V. B. (1991), (IEEE Computer Society Press, Los Alamitos, Calif.)). A marker gene selection process was performed by feeding the k-NN algorithm only the features with higher correlation with the target class. In this version of the algorithm the weight of each of the k neighbors was weighted according to 1/d.

The cross-validation step was repeated for each sample and the errors were tallied. A random 8-class classifier would be expected to give an error rate of 100-(100/8), or 87.5%. For the initial validation of clusters, classifiers were built with various numbers of marker genes selected from the 675-gene set that was used for hierarchical clustering. The best model used 100 genes (13% overall error); however, models using 75-200 genes performed with less than 20% overall error.

For testing whether the cluster definitions were highly dependent on the 675-gene set, classifiers were built from the remaining 11,925 genes. The genes were passed through a variation filter and marker genes were selected as above. A 100-gene model gave an overall error rate of 26%, with the classes that represent clusters performing better than the “other” class.

Kaplan-Meier Analysis and Permutation Testing.

Kaplan-Meier curves were generated using standard functions in S-PLUS package (Venables, W. N. & Ripley, B. D. (1998) Modern applied statistics with S-PLUS (Springer, Berlin)). Only 125 adenocarcinoma samples were used with survival information from adenocarcinoma samples. For each cluster, survival within-clusters was compared to the out-of-cluster group using the two-sample comparison based on the corresponding two K-M curves. In this way 5 K-M plots was obtained for each cluster, of which two plots have significant P-values for the comparison of the two curves, namely cluster 2 (C2, P=0.00476) and cluster 4 (C4, P=0.049). A similar analysis performed for stage I patient samples was statistically non-significant for all clusters. The small sample size (n=4) is a possible factor in the non-significance of the result for Stage I C2 patients.

These apparently significant P-values have a bias because of multiple hypothesis testing. To test for this selection bias, the cluster labels were randomly permuted among the samples and K-M significance, for each cluster, the within-cluster and out-of-cluster K-M curves and the corresponding P-values were re-computed. This randomization was repeated 1000 times. The 1000 sets of P-values were used to construct the null distributions for the test statistic T1=the smallest P-value among 5 clusters. From the 1000 permutations, the P-values for T1=0.044. This P-value is a reasonable assessment of the significance of outcome differences for the cluster C2 (FIG. 1). This statistical evidence supports the predictive value of C2 on survival.

### Example 3

### Gene Markers for Different Lung Cancers and Adenocarcinoma Sub-Classes

Expression data were preprocessed by setting a minimal level of 10 units and only genes that showed 5-fold change across the data set were analyzed further. Genes correlated with a particular cluster labels (e.g. “c0” or “colon”) were identified by sorting all of the genes on the array according the signal-to-noise statistic (mu_c0−mu_others)/(sd_c0+sd_others), where mu and sd represent the mean and standard deviation of expression, respectively, for each class.

Permutation of the column (sample) labels was performed to compare these correlations to what would be expected by chance. The top signal-to-noise scores for top marker genes were compared and compared with the corresponding ones for random permutation version of the cluster labels. 1000 random permutations were used to build histograms for the top marker, the second best, etc. Based on this histogram the 0.1% significance levels were estimated as compared with the values obtained for the real dataset. This test helps to assess the statistical significance of gene markers in terms of target class-correlations.

Included in the list of genes are those that exceed the 0.1% significance level for each cluster. For those clusters (colon, normal, C4) for which the lists are very long, only the top 200 genes are shown. The following Tables 1-8 present genes for the C₁-C₄ subclasses, normal, colorectal metastases, C0, and other subclasses. (The s2n_obs is the observed signal to noise value; the non_norm_list is the Affymetrix reference identifier; the LL_num is the LocusLink identifier; and Desc is the description of the gene or gene product.

TABLE 1
C1 Markers
Class C1

| | | | | | UNIGENE | | |
|---|---|---|---|---|---|---|---|
| | | | | | (as of | | Desc |
| | | Perm | | GB/TIGR | summer | | (unigene/locuslink |
| | s2n_obs | 0.1% | non_norm_list | Identifier | 2001) | LL_num | or affy) |
| 1 | 1.29 | 1.024 | 36457_at | U10860 | Hs.5398 | 8833 | guanine |
| | | | | | | | monphosphate |
| | | | | | | | synthetase |
| 2 | 1.25 | 0.865 | 40117_at | D84557 | Hs.155462 | 4175 | minichromosome |
| | | | | | | | maintenance |
| | | | | | | | deficient (mis5, S. |
| | | | | | | | pombe) 6 |
| 3 | 1.22 | 0.797 | 37337_at | AI803447 | Hs.77496 | 6637 | small nuclear |
| | | | | | | | ribonucleoprotein |
| | | | | | | | polypeptide G |
| 4 | 1.18 | 0.770 | 1055_g_at | M87339 | Hs.35120 | 5984 | replication factor C |
| | | | | | | | (activator 1) 4 |
| | | | | | | | (37 kD) |
| 5 | 1.18 | 0.767 | 41547_at | AF047472 | Hs.40323 | 9184 | BUB3 (budding |
| | | | | | | | uninhibited by |
| | | | | | | | benzimidazoles 3, |
| | | | | | | | yeast) homolog |
| 6 | 1.17 | 0.763 | 38840_s_at | L10678 | Hs.91747 | 5217 | profilin 2 |
| 7 | 1.12 | 0.757 | 38065_at | X62534 | Hs.80684 | 3148 | high-mobility |
| | | | | | | | group (nonhistone |
| | | | | | | | chromosomal) |
| | | | | | | | protein 2 |
| 8 | 1.11 | 0.754 | 709_at | J00314 | Hs.336780 | 7280 | tubulin, beta |
| | | | | | | | polypeptide |
| 9 | 1.1 | 0.739 | 41583_at | AC004770 | Hs.4756 | 2237 | flap structure- |
| | | | | | | | specific |
| | | | | | | | endonuclease 1 |
| 10 | 1.06 | 0.731 | 40195_at | X14850 | Hs.147097 | 3014 | H2A histone |
| | | | | | | | family, member X |
| 11 | 1.05 | 0.728 | 39109_at | AB024704 | Hs.9329 | 22974 | chromosome 20 |
| | | | | | | | open reading frame |
| | | | | | | | 1 |
| 12 | 1.05 | 0.727 | 207_at | M86752 | Hs.75612 | 10963 | stress-induced- |
| | | | | | | | phosphoprotein 1 |
| | | | | | | | (Hsp70/Hsp90- |
| | | | | | | | organizing protein) |
| 13 | 1.05 | 0.722 | 1884_s_at | M15796 | Hs.78996 | 5111 | proliferating cell |
| | | | | | | | nuclear antigen |
| 14 | 1.04 | 0.716 | 34763_at | AF020043 | Hs.24485 | 9126 | chondroitin sulfate |
| | | | | | | | proteoglycan 6 |
| | | | | | | | (bamacan) |
| 15 | 1.02 | 0.715 | 40619_at | M91670 | Hs.174070 | 27338 | ubiquitin carrier |
| | | | | | | | protein |
| 16 | 1.01 | 0.715 | 1824_s_at | J05614 | | | proliferating cell |
| | | | | | | | nuclear antigen |
| | | | | | | | (PCNA) |
| 17 | 1.01 | 0.714 | 572_at | M86699 | Hs.169840 | 7272 | TTK protein |
| | | | | | | | kinase |
| 18 | 1 | 0.711 | 151_s_at | V00599 | Hs.179661 | 2280 | V00599 |
| | | | | | | | /FEATURE = mRNA |
| | | | | | | | /DEFINITION = HS |
| | | | | | | | TUB2 Human |
| | | | | | | | mRNA fragment |
| | | | | | | | encoding beta- |
| | | | | | | | tubulin. (from |
| | | | | | | | clone D-beta-1) |
| 19 | 1 | 0.708 | 1803_at | X05360 | Hs.184572 | 983 | cell division cycle |
| | | | | | | | 2, G1 to S and G2 |
| | | | | | | | to M |
| 20 | 0.99 | 0.706 | 1515_at | HG4074- | | | Rad2 |
| | | | | HT4344 | | | |
| 21 | 0.98 | 0.704 | 34791_at | X52882 | Hs.4112 | 6950 | t-complex 1 |
| 22 | 0.97 | 0.702 | 40690_at | X54942 | Hs.83758 | 1164 | CDC28 protein |
| | | | | | | | kinase 2 |
| 23 | 0.96 | 0.700 | 40697_at | X51688 | Hs.85137 | 890 | cyclin A2 |
| 24 | 0.96 | 0.696 | 37686_s_at | Y09008 | Hs.78853 | 7374 | uracil-DNA |
| | | | | | | | glycosylase |
| 25 | 0.96 | 0.693 | 982_at | X74795 | Hs.77171 | 4174 | minichromosome |
| | | | | | | | maintenance |
| | | | | | | | deficient (S. |
| | | | | | | | cerevisiae) 5 (cell |
| | | | | | | | division cycle 46) |
| 26 | 0.95 | 0.692 | 1505_at | D00596 | Hs.82962 | 7298 | thymidylate |
| | | | | | | | synthetase |
| 27 | 0.94 | 0.690 | 38992_at | X64229 | Hs.110713 | 7913 | DEK oncogene |
| | | | | | | | (DNA binding) |
| 28 | 0.94 | 0.690 | 33255_at | M97856 | Hs.243886 | 4678 | nuclear |
| | | | | | | | autoantigenic |
| | | | | | | | sperm protein |
| | | | | | | | (histone-binding) |
| 29 | 0.94 | 0.688 | 36813_at | U96131 | Hs.6566 | 9319 | thyroid hormone |
| | | | | | | | receptor interactor |
| | | | | | | | 13 |
| 30 | 0.93 | 0.684 | 34882_at | Y12065 | Hs.296585 | 10528 | nucleolar protein |
| | | | | | | | (KKE/D repeat) |
| 31 | 0.91 | 0.684 | 34715_at | U74612 | Hs.239 | 2305 | forkhead box M1 |
| 32 | 0.9 | 0.683 | 674_g_at | J04031 | Hs.172665 | 4522 | methylenetetra- |
| | | | | | | | hydrofolate |
| | | | | | | | dehydrogenase |
| | | | | | | | (NADP+ |
| | | | | | | | dependent), |
| | | | | | | | methenyltetra- |
| | | | | | | | hydrofolate |
| | | | | | | | cyclohydrolase, |
| | | | | | | | formyltetrahydro- |
| | | | | | | | folate synmetase |
| 33 | 0.9 | 0.680 | 39337_at | M37583 | Hs.119192 | 3015 | H2A histone |
| | | | | | | | family, member Z |
| 34 | 0.89 | 0.679 | 41756_at | AJ010842 | Hs.18259 | 11321 | XPA binding |
| | | | | | | | protein 1; putative |
| | | | | | | | ATP (GTP)- |
| | | | | | | | binding protein |
| 35 | 0.89 | 0.678 | 40417_at | D43950 | | | chaperonin |
| | | | | | | | containing TCP1, |
| | | | | | | | subunit 5 (epsilon) |
| 36 | 0.89 | 0.677 | 571_at | M86667 | Hs.179662 | 4673 | nucleosome |
| | | | | | | | assembly protein |
| | | | | | | | 1-like 1 |
| 37 | 0.89 | 0.676 | 38804_at | AF053641 | Hs.90073 | 1434 | chromosome |
| | | | | | | | segregation 1 |
| | | | | | | | (yeast homolog)- |
| | | | | | | | like |
| 38 | 0.88 | 0.675 | 37304_at | U35451 | Hs.77254 | 10951 | chromobox |
| | | | | | | | homolog 1 |
| | | | | | | | (Drosophila HP1 |
| | | | | | | | beta) |
| 39 | 0.88 | 0.674 | 34383_at | AB014458 | Hs.35086 | 7398 | ubiquitin specific |
| | | | | | | | protease 1 |
| 40 | 0.87 | 0.674 | 2003_s_at | U28946 | Hs.3248 | 2956 | mutS (E. coli) |
| | | | | | | | homolog 6 |
| 41 | 0.87 | 0.673 | 40407_at | U28386 | Hs.159557 | 3838 | karyopherin alpha |
| | | | | | | | 2 (RAG cohort 1, |
| | | | | | | | importin alpha 1) |
| 42 | 0.87 | 0.672 | 40041_at | AF017790 | Hs.58169 | 10403 | highly expressed in |
| | | | | | | | cancer, rich in |
| | | | | | | | leucine heptad |
| | | | | | | | repeats |
| 43 | 0.85 | 0.668 | 41375_at | AJ245416 | Hs.103106 | 57819 | U6 snRNA- |
| | | | | | | | associated Sm-like |
| | | | | | | | protein |
| 44 | 0.85 | 0.666 | 1985_s_at | X73066 | Hs.118638 | 4830 | non-metastatic |
| | | | | | | | cells 1, protein |
| | | | | | | | (NM23A) |
| | | | | | | | expressed in |
| 45 | 0.85 | 0.664 | 36987_at | M94362 | Hs.334709 | 3999 | lamin B2 |
| 46 | 0.84 | 0.663 | 1782_s_at | M31303 | Hs.81915 | 3925 | leukemia- |
| | | | | | | | associated |
| | | | | | | | phosphoprotein |
| | | | | | | | p18 (stathmin) |
| 47 | 0.84 | 0.659 | 35699_at | AF053306 | Hs.36708 | 701 | budding |
| | | | | | | | uninhibited by |
| | | | | | | | benzimidazoles 1 |
| | | | | | | | (yeast homolog), |
| | | | | | | | beta |
| 48 | 0.84 | 0.658 | 38414_at | U05340 | Hs.82906 | 991 | CDC20 (cell |
| | | | | | | | division cycle 20, |
| | | | | | | | S. cerevisiae, |
| | | | | | | | homolog) |
| 49 | 0.84 | 0.657 | 35218_at | AF022385 | Hs.28866 | 11235 | programmed cell |
| | | | | | | | death 10 |
| 50 | 0.84 | 0.656 | 40726_at | U37426 | Hs.8878 | 3832 | kinesin-like 1 |
| 51 | 0.83 | 0.653 | 1136_at | L16991 | Hs.79006 | 1841 | deoxythymidylate |
| | | | | | | | kinase |
| | | | | | | | (thymidylate |
| | | | | | | | kinase) |
| 52 | 0.83 | 0.652 | 36098_at | M72709 | Hs.73737 | 6426 | splicing factor, |
| | | | | | | | arginine/serine- |
| | | | | | | | rich 1 (splicing |
| | | | | | | | factor 2, alternate |
| | | | | | | | splicing factor) |
| 53 | 0.83 | 0.650 | 38350_f_at | AF005392 | Hs.98102 | 7278 | tubulin, alpha 2 |
| 54 | 0.83 | 0.649 | 39374_at | AL022325 | Hs.122552 | 51512 | hypothetical |
| | | | | | | | protein FLJ10140 |
| 55 | 0.83 | 0.649 | 34314_at | X59543 | Hs.2934 | 6240 | ribonucleotide |
| | | | | | | | reductase M1 |
| | | | | | | | polypeptide |
| 56 | 0.83 | 0.648 | 38473_at | M63180 | Hs.84131 | 6897 | threonyl-tRNA |
| | | | | | | | synthetase |
| 57 | 0.83 | 0.647 | 1945_at | M25753 | Hs.23960 | 891 | cyclin B1 |
| 58 | 0.83 | 0.646 | 37347_at | AA926959 | Hs.77550 | 84722 | hypothetical |
| | | | | | | | protein MGC1780 |
| 59 | 0.82 | 0.645 | 40587_s_at | AF054186 | Hs.298581 | 9521 | eukaryotic |
| | | | | | | | translation |
| | | | | | | | elongation factor 1 |
| | | | | | | | epsilon 1 |
| 60 | 0.82 | 0.645 | 41342_at | D38076 | Hs.24763 | 5902 | RAN binding |
| | | | | | | | protein 1 |
| 61 | 0.82 | 0.645 | 860_at | U03911 | Hs.78934 | 4436 | mutS (E. coli) |
| | | | | | | | homolog 2 (colon |
| | | | | | | | cancer, |
| | | | | | | | nonpolyposis type |
| | | | | | | | 1) |
| 62 | 0.82 | 0.643 | 41569 at | AI680675 | Hs.44131 | 23234 | KIAA0974 protein |
| 63 | 0.82 | 0.642 | 32610_at | X93510 | Hs.79691 | 8572 | LIM domain |
| | | | | | | | protein |
| 64 | 0.81 | 0.639 | 33247_at | U86782 | Hs.178761 | 10213 | 26S proteasome- |
| | | | | | | | associated pad1 |
| | | | | | | | homolog |
| 65 | 0.81 | 0.638 | 32530_at | X56468 | Hs.74405 | 10971 | tyrosine 3- |
| | | | | | | | monooxygenase/ |
| | | | | | | | tryptophan 5- |
| | | | | | | | monooxygenase |
| | | | | | | | activation protein, |
| | | | | | | | theta polypeptide |
| 66 | 0.81 | 0.638 | 1854_at | X13293 | Hs.179718 | 4605 | v-myb avian |
| | | | | | | | myeloblastosis |
| | | | | | | | viral oncogene |
| | | | | | | | homolog-like 2 |
| 67 | 0.81 | 0.637 | 37333_at | X63692 | Hs.77462 | 1786 | DNA (cytosine-5-)- |
| | | | | | | | methyltransferase |
| | | | | | | | 1 |
| 68 | 0.8 | 0.637 | 318_at | D64142 | Hs.109804 | 8971 | H1 histone family, |
| | | | | | | | member X |
| 69 | 0.8 | 0.636 | 418_at | X65550 | Hs.80976 | 4288 | antigen identified |
| | | | | | | | by monoclonal |
| | | | | | | | antibody Ki-67 |
| 70 | 0.8 | 0.635 | 38116_at | D14657 | Hs.81892 | 9768 | KIAA0101 gene |
| | | | | | | | product |
| 71 | 0.8 | 0.634 | 40638_at | X70944 | Hs.180610 | 6421 | splicing factor |
| | | | | | | | proline/glutamine |
| | | | | | | | rich |
| | | | | | | | (polypyrimidine |
| | | | | | | | tract-binding |
| | | | | | | | protein-associated) |
| 72 | 0.8 | 0.633 | 36913_at | U75679 | Hs.75257 | 7884 | Hairpin binding |
| | | | | | | | protein, histone |
| 73 | 0.79 | 0.631 | 36171_at | AI521453 | Hs.74861 | 10923 | activated RNA |
| | | | | | | | polymerase II |
| | | | | | | | transcription |
| | | | | | | | cofactor 4 |
| 74 | 0.79 | 0.631 | 38251_at | AI127424 | Hs.90318 | 4632 | myosin, light |
| | | | | | | | polypeptide 1, |
| | | | | | | | alkali; skeletal, fast |
| 75 | 0.79 | 0.631 | 32214_at | AF003938 | Hs.18792 | 9352 | thioredoxin-like, |
| | | | | | | | 32 kD |
| 76 | 0.79 | 0.630 | 35312_at | D21063 | Hs.57101 | 4171 | minichromosome |
| | | | | | | | maintenance |
| | | | | | | | deficient (S. |
| | | | | | | | cerevisiae) 2 |
| | | | | | | | (mitotin) |
| 77 | 0.79 | 0.630 | 35995 at | AF067656 | Hs.42650 | 11130 | ZW10 interactor |
| 78 | 0.79 | 0.626 | 39677_at | D80008 | Hs.36232 | 9837 | KIAA0186 gene |
| | | | | | | | product |
| 79 | 0.78 | 0.624 | 38031_at | D21853 | Hs.79768 | 9775 | KIAA0111 gene |
| | | | | | | | product |
| 80 | 0.78 | 0.624 | 34327_at | Z46606 | | | HLTF gene for |
| | | | | | | | helicase-like |
| | | | | | | | transcription factor |
| | | | | | | | /cds = UNKNOWN |
| | | | | | | | /gb = Z46606 |
| | | | | | | | /gi = 575250 |
| | | | | | | | /ug = Hs.3068 |
| | | | | | | | /len = 5439 |
| 81 | 0.78 | 0.623 | 41322_s_at | AI816034 | Hs.23990 | 55651 | nucleolar protein |
| | | | | | | | family A, member |
| | | | | | | | 2 (H/ACA small |
| | | | | | | | nucleolar RNPs) |
| 82 | 0.78 | 0.622 | 36941_at | U16954 | Hs.75823 | 10962 | ALL1 -fused gene |
| | | | | | | | from chromosome |
| | | | | | | | 1q |
| 83 | 0.78 | 0.621 | 37228_at | U01038 | Hs.77597 | 5347 | polo (Drosophia)- |
| | | | | | | | like kinase |
| 84 | 0.78 | 0.620 | 140_s_at | U68063 | Hs.30035 | 6434 | splicing factor, |
| | | | | | | | arginine/serine- |
| | | | | | | | rich (transformer 2 |
| | | | | | | | Drosophila |
| | | | | | | | homolog) 10 |
| 85 | 0.77 | 0.620 | 149_at | U90426 | Hs.179606 | 10212 | nuclear RNA |
| | | | | | | | helicase, DECD |
| | | | | | | | variant of DEAD |
| | | | | | | | box family |
| 86 | 0.77 | 0.620 | 349_g_at | D14678 | Hs.20830 | 3833 | kinesin-like 2 |
| 87 | 0.77 | 0.619 | 1599_at | L25876 | Hs.84113 | 1033 | cyclin-dependent |
| | | | | | | | kinase inhibitor 3 |
| | | | | | | | (CDK2-associated |
| | | | | | | | dual specificity |
| | | | | | | | phosphatase) |
| 88 | 0.77 | 0.619 | 39056_at | X53793 | Hs.117950 | 10606 | multifunctional |
| | | | | | | | polypeptide similar |
| | | | | | | | to SAICAR |
| | | | | | | | synthetase and |
| | | | | | | | AIR carboxylase |
| 89 | 0.77 | 0.618 | 32594_at | AF026291 | Hs.79150 | 10575 | chaperonin |
| | | | | | | | containing TCP1, |
| | | | | | | | subunit 4 (delta) |
| 90 | 0.77 | 0.618 | 37985_at | L37747 | | | lamin B1 |
| 91 | 0.77 | 0.618 | 584_s_at | M30938 | Hs.84981 | 7520 | X-ray repair |
| | | | | | | | complementing |
| | | | | | | | defective repair in |
| | | | | | | | Chinese hamster |
| | | | | | | | cells 5 (double- |
| | | | | | | | strand-break |
| | | | | | | | rejoining; Ku |
| | | | | | | | autoantigen, 80 kD) |
| 92 | 0.77 | 0.618 | 34659_at | AB018334 | Hs.23255 | 9631 | nucleoporin 155 kD |
| 93 | 0.77 | 0.616 | 39812_at | X79865 | Hs.109059 | 6182 | mitochondrial |
| | | | | | | | ribosomal protein |
| | | | | | | | L12 |
| 94 | 0.77 | 0.615 | 41403_at | AI032612 | Hs.105465 | 6636 | small nuclear |
| | | | | | | | ribonucleoprotein |
| | | | | | | | polypeptide F |
| 95 | 0.76 | 0.615 | 33252_at | D38073 | Hs.179565 | 4172 | minichromosome |
| | | | | | | | maintenance |
| | | | | | | | deficient (S. |
| | | | | | | | cerevisiae) 3 |
| 96 | 0.76 | 0.614 | 37738_g_at | D25547 | Hs.79137 | 5110 | protein-L- |
| | | | | | | | isoaspartate (D- |
| | | | | | | | aspartate) O- |
| | | | | | | | methyltransferase |
| 97 | 0.76 | 0.614 | 35916_s_at | AA877215 | | | cDNA, 3 end |
| 98 | 0.75 | 0.613 | 32843_s_at | M30448 | | | casein kinase 2, |
| | | | | | | | beta polypeptide |
| 99 | 0.75 | 0.613 | 1674_at | M15990 | Hs.194148 | 7525 | v-yes-1 |
| | | | | | | | Yamaguchi |
| | | | | | | | sarcoma viral |
| | | | | | | | oncogene homolog |
| | | | | | | | 1 |
| 100 | 0.74 | 0.611 | 40842_at | M60784 | | | small nuclear |
| | | | | | | | ribonucleoprotein |
| | | | | | | | polypeptide A |
| 101 | 0.74 | 0.610 | 38847_at | D79997 | Hs.184339 | 9833 | KIAA0175 gene |
| | | | | | | | product |
| 102 | 0.74 | 0.609 | 39965_at | AI570572 | Hs.45002 | 5881 | ras-related C3 |
| | | | | | | | botulinum toxin |
| | | | | | | | substrate 3 (rho |
| | | | | | | | family, small GTP |
| | | | | | | | binding protein |
| | | | | | | | Rac3) |
| 103 | 0.74 | 0.609 | 351_f_at | D28423 | | | pre-mRNA |
| | | | | | | | splicing factor |
| | | | | | | | SRp20, 5″UTR |
| 104 | 0.73 | 0.607 | 36135_at | U86602 | Hs.74407 | 10969 | nucleolar protein |
| | | | | | | | p40; homolog of |
| | | | | | | | yeast EBNA1- |
| | | | | | | | binding protein |
| 105 | 0.73 | 0.607 | 39076_s_at | AI991040 | Hs.334879 | 10589 | DR1-associated |
| | | | | | | | protein 1 (negative |
| | | | | | | | cofactor 2 alpha) |
| 106 | 0.73 | 0.606 | 34878_at | AB019987 | Hs.50758 | 10051 | SMC4 (structural |
| | | | | | | | maintenance of |
| | | | | | | | chromosomes 4, |
| | | | | | | | yeast)-like 1 |
| 107 | 0.73 | 0.604 | 41855_at | AF030424 | Hs.13340 | 8520 | histone |
| | | | | | | | acetyltransferase 1 |
| 108 | 0.73 | 0.604 | 38792_at | AD001528 | Hs.89718 | 6611 | spermine synthase |
| 109 | 0.72 | 0.602 | 38123_at | D14878 | Hs.82043 | 8872 | D123 gene product |
| 110 | 0.72 | 0.602 | 40145_at | AI375913 | Hs.156346 | 7153 | topoisomerase |
| | | | | | | | (DNA) II alpha |
| | | | | | | | (170 kD) |
| 111 | 0.72 | 0.601 | 39262_at | U79266 | Hs.23642 | 29901 | protein predicted |
| | | | | | | | by clone 23627 |
| 112 | 0.72 | 0.600 | 36107_at | AA845575 | Hs.73851 | 522 | ATP synthase, H+ |
| | | | | | | | transporting, |
| | | | | | | | mitochondrial F0 |
| | | | | | | | complex, subunit |
| | | | | | | | F6 |
| 113 | 0.72 | 0.599 | 37305_at | U61145 | Hs.77256 | 2146 | enhancer of zeste |
| | | | | | | | (Drosophila) |
| | | | | | | | homolog 2 |
| 114 | 0.72 | 0.599 | 34380_at | AC004472 | Hs.3439 | 30968 | stomatin-like 2 |
| 115 | 0.72 | 0.599 | 276_at | L08069 | Hs.94 | 3301 | heat shock protein, |
| | | | | | | | DNAJ-like 2 |
| 116 | 0.72 | 0.599 | 34795_at | U84573 | Hs.41270 | 5352 | procollagen-lysine, |
| | | | | | | | 2-oxoglutarate 5- |
| | | | | | | | dioxygenase |
| | | | | | | | (lysine |
| | | | | | | | hydroxylase) 2 |
| 117 | 0.71 | 0.599 | 39969_at | AA255502 | Hs.46423 | 8364 | H4 histone family, |
| | | | | | | | member G |
| 118 | 0.71 | 0.599 | 32844_at | AF104913 | Hs.211568 | 1981 | eukaryotic |
| | | | | | | | translation |
| | | | | | | | initiation factor 4 |
| | | | | | | | gamma, 1 |
| 119 | 0.71 | 0.599 | 41407_at | L03411 | Hs.106061 | 7936 | RD RNA-binding |
| | | | | | | | protein |
| 120 | 0.71 | 0.598 | 39759_at | AL031781 | Hs.15020 | 9444 | homolog of mouse |
| | | | | | | | quaking QKI (KH |
| | | | | | | | domain RNA |
| | | | | | | | binding protein) |
| 121 | 0.71 | 0.598 | 35364_at | U50939 | Hs.61828 | 8883 | amyloid beta |
| | | | | | | | precursor protein- |
| | | | | | | | binding protein 1, |
| | | | | | | | 59 kD |
| 122 | 0.71 | 0.598 | 36812_at | U92715 | Hs.6564 | 8412 | breast cancer anti- |
| | | | | | | | estrogen resistance |
| | | | | | | | 3 |
| 123 | 0.71 | 0.598 | 36837_at | U63743 | Hs.69360 | 11004 | kinesin-like 6 |
| | | | | | | | (mitotic |
| | | | | | | | centromere- |
| | | | | | | | associated kinesin) |
| 124 | 0.71 | 0.597 | 471_f_at | U47634 | Hs.159154 | 10381 | tubulin, beta, 4 |
| 125 | 0.71 | 0.597 | 40879_at | AB014599 | Hs.330988 | 23299 | KIAA0699 protein |
| 126 | 0.71 | 0.596 | 947_at | D55716 | Hs.77152 | 4176 | minichromosome |
| | | | | | | | maintenance |
| | | | | | | | deficient (S. |
| | | | | | | | cerevisiae) 7 |
| 127 | 0.71 | 0.595 | 157_at | U65011 | Hs.30743 | 23532 | preferentially |
| | | | | | | | expressed antigen |
| | | | | | | | in melanoma |
| 128 | 0.7 | 0.593 | 35200_at | X92518 | Hs.2726 | 8091 | high-mobility |
| | | | | | | | group (nonhistone |
| | | | | | | | chromosomal) |
| | | | | | | | protein isoform I-C |
| 129 | 0.7 | 0.592 | 32194_at | M37197 | Hs.184760 | 10153 | CCAAT-box- |
| | | | | | | | binding |
| | | | | | | | transcription factor |
| 130 | 0.7 | 0.592 | 39173_at | X56597 | Hs.99853 | 2091 | fibrillarin |
| 131 | 0.7 | 0.590 | 1840_g_at | HG1112- | | | Ras-Like Protein |
| | | | | HT1112 | | | Tc4 |
| 132 | 0.7 | 0.588 | 37739_at | M86737 | Hs.79162 | 6749 | structure specific |
| | | | | | | | recognition protein |
| | | | | | | | 1 |
| 133 | 0.7 | 0.587 | 34510_at | AF070552 | Hs.122908 | 81620 | DNA replication |
| | | | | | | | factor |
| 134 | 0.7 | 0.585 | 36536_at | AF070614 | Hs.61490 | 29970 | schwannomin |
| | | | | | | | interacting protein |
| | | | | | | | 1 |
| 135 | 0.7 | 0.583 | 36863_at | AF032862 | Hs.72550 | 3161 | hyaluronan- |
| | | | | | | | mediated motility |
| | | | | | | | receptor |
| | | | | | | | (RHAMM) |
| 136 | 0.69 | 0.583 | 34790_at | S70154 | Hs.278544 | 39 | acetyl-Coenzyme |
| | | | | | | | A acetyltransferase |
| | | | | | | | 2 (acetoacetyl |
| | | | | | | | Coenzyme A |
| | | | | | | | thiolase) |
| 137 | 0.69 | 0.583 | 527_at | U14518 | Hs.1594 | 1058 | centromere protein |
| | | | | | | | A (17 kD) |
| 138 | 0.69 | 0.581 | 38679_g_at | AA733050 | Hs.1066 | 6635 | small nuclear |
| | | | | | | | ribonucleoprotein |
| | | | | | | | polypeptide E |
| 139 | 0.69 | 0.581 | 39984_g_at | U73704 | Hs.49105 | 11146 | FKBP-associated |
| | | | | | | | protein |
| 140 | 0.68 | 0.581 | 40610_at | AI743507 | Hs.173518 | 51663 | likely ortholog of |
| | | | | | | | mouse zinc finger |
| | | | | | | | protein Zfr |
| 141 | 0.68 | 0.581 | 39792_at | AF000364 | Hs.15265 | 10236 | heterogeneous |
| | | | | | | | nuclear |
| | | | | | | | ribonucleoprotein |
| | | | | | | | R |
| 142 | 0.68 | 0.579 | 33266_at | AF015254 | Hs.180655 | 9212 | serine/threonine |
| | | | | | | | kinase 12 |
| 143 | 0.68 | 0.578 | 31858_at | X07315 | Hs.151734 | 10204 | nuclear transport |
| | | | | | | | factor 2 (placental |
| | | | | | | | protein 15) |
| 144 | 0.68 | 0.578 | 32340_s_at | M85234 | Hs.74497 | 4904 | nuclease sensitive |
| | | | | | | | element binding |
| | | | | | | | protein 1 |
| 145 | 0.68 | 0.577 | 34099_f_at | W26056 | Hs.343569 | | cDNA |
| 146 | 0.68 | 0.577 | 831_at | U28042 | Hs.41706 | 1662 | DEAD/H (Asp- |
| | | | | | | | Glu-Ala-Asp/His) |
| | | | | | | | box polypeptide 10 |
| | | | | | | | (RNA helicase) |
| 147 | 0.68 | 0.576 | 37945_at | U91316 | Hs.8679 | 11332 | cytosolic acyl |
| | | | | | | | coenzyme A |
| | | | | | | | thioester hydrolase |
| 148 | 0.68 | 0.576 | 33035_at | AL021397 | Hs.137576 | 26514 | ribosomal protein |
| | | | | | | | L34 pseudogene 1 |
| 149 | 0.68 | 0.575 | 32120_at | AF063308 | Hs.16244 | 10615 | mitotic spindle |
| | | | | | | | coiled-coil related |
| | | | | | | | protein |
| 150 | 0.68 | 0.575 | 36104_at | AA526497 | Hs.73818 | 7388 | ubiquinol- |
| | | | | | | | cytochrome c |
| | | | | | | | reductase hinge |
| | | | | | | | protein |
| 151 | 0.67 | 0.575 | 32548_at | L24804 | Hs.278270 | 10728 | unactive |
| | | | | | | | progesterone |
| | | | | | | | receptor, 23 kD |
| 152 | 0.67 | 0.574 | 36872_at | AL120559 | Hs.7351 | 10776 | cyclic AMP |
| | | | | | | | phosphoprotein, 19 |
| | | | | | | | kD |
| 153 | 0.67 | 0.573 | 38634_at | M11433 | Hs.101850 | 5947 | retinol-binding |
| | | | | | | | protein 1, cellular |
| 154 | 0.67 | 0.573 | 37683_at | D80012 | Hs.78829 | 9100 | ubiquitin specific |
| | | | | | | | protease 10 |
| 155 | 0.67 | 0.573 | 33127_at | U89942 | Hs.83354 | 4017 | lysyl oxidase-like |
| | | | | | | | 2 |
| 156 | 0.67 | 0.572 | 41401_at | U57646 | Hs.10526 | 1466 | cysteine and |
| | | | | | | | glycine-rich |
| | | | | | | | protein 2 |
| 157 | 0.67 | 0.572 | 40074_at | X16396 | Hs.154672 | 10797 | methylene |
| | | | | | | | tetrahydrofolate |
| | | | | | | | dehydrogenase |
| | | | | | | | (NAD+ |
| | | | | | | | dependent), |
| | | | | | | | methenyltetra- |
| | | | | | | | hydrofolate |
| | | | | | | | cyclohydrolase |
| 158 | 0.66 | 0.572 | 41600_at | U59435 | Hs.5181 | 5036 | proliferation- |
| | | | | | | | associated 2G4, |
| | | | | | | | 38 kD |
| 159 | 0.66 | 0.571 | 1449_at | D00763 | Hs.251531 | 5685 | proteasome |
| | | | | | | | (prosome, |
| | | | | | | | macropain) |
| | | | | | | | subunit, alpha |
| | | | | | | | type, 4 |
| 160 | 0.66 | 0.570 | 37046_at | AI246726 | Hs.76913 | 5686 | proteasome |
| | | | | | | | (prosome, |
| | | | | | | | macropain) |
| | | | | | | | subunit, alpha |
| | | | | | | | type, 5 |
| 161 | 0.66 | 0.570 | 34814_at | AL041443 | Hs.4311 | 10054 | SUMO-1 |
| | | | | | | | activating enzyme |
| | | | | | | | subunit 2 |
| 162 | 0.66 | 0.570 | 32615_at | J05032 | Hs.80758 | 1615 | aspartyl-tRNA |
| | | | | | | | synthetase |
| 163 | 0.66 | 0.569 | 39086_g_at | AA768912 | Hs.923 | 6742 | single-stranded |
| | | | | | | | DNA-binding |
| | | | | | | | protein 1 |
| 164 | 0.65 | 0.569 | 39747_at | U52427 | Hs.14839 | 5436 | polymerase (RNA) |
| | | | | | | | II (DNA directed) |
| | | | | | | | polypeptide G |
| 165 | 0.65 | 0.568 | 39009_at | N98670 | | | cDNA, 5 end |
| 166 | 0.65 | 0.568 | 40124_at | Y18418 | Hs.272822 | 8607 | RuvB (E coli |
| | | | | | | | homolog)-like 1 |
| 167 | 0.65 | 0.568 | 32730_at | AL080059 | Hs.173094 | 85453 | Homo sapiens |
| | | | | | | | mRNA for |
| | | | | | | | KIAA1750 |
| | | | | | | | protein, partial cds |
| 168 | 0.64 | 0.567 | 38662_at | AL047596 | Hs.306117 | 23152 | KIAA0306 protein |
| 169 | 0.64 | 0.567 | 33679_f_at | X02344 | Hs.251653 | 10383 | tubulin, beta, 2 |
| 170 | 0.64 | 0.567 | 37302_at | U30872 | Hs.77204 | 1063 | centromere protein |
| | | | | | | | F (350/400 kD, |
| | | | | | | | mitosin) |
| 171 | 0.64 | 0.566 | 39704_s_at | L17131 | Hs.139800 | 3159 | high-mobility |
| | | | | | | | group (nonhistone |
| | | | | | | | chromosomal) |
| | | | | | | | protein isoforms I |
| | | | | | | | and Y |
| 172 | 0.64 | 0.565 | 131_at | X83928 | Hs.83126 | 6882 | TATA box binding |
| | | | | | | | protein (TBP)- |
| | | | | | | | associated factor, |
| | | | | | | | RNA polymerase |
| | | | | | | | II, I, 28 kD |
| 173 | 0.64 | 0.565 | 40779_at | U59919 | Hs.171374 | 22920 | smg GDS- |
| | | | | | | | ASSOCIATED |
| | | | | | | | PROTEIN |
| 174 | 0.64 | 0.564 | 38114_at | D38551 | Hs.81848 | 5885 | RAD21 (S. |
| | | | | | | | pombe) homolog |
| 175 | 0.64 | 0.564 | 32850_at | Z25535 | Hs.211608 | 9972 | nucleoporin 153 kD |
| 176 | 0.64 | 0.564 | 1250_at | U47077 | Hs.155637 | 5591 | protein kinase, |
| | | | | | | | DNA-activated, |
| | | | | | | | catalytic |
| | | | | | | | polypeptide |
| 177 | 0.64 | 0.564 | 37345_at | AF013759 | Hs.7753 | 813 | calumenin |
| 178 | 0.64 | 0.563 | 37293_at | D43948 | Hs.76989 | 9793 | KIAA0097 gene |
| | | | | | | | product |
| 179 | 0.64 | 0.563 | 40418_at | X74262 | Hs.16003 | 5928 | retinoblastoma- |
| | | | | | | | binding protein 4 |
| 180 | 0.64 | 0.562 | 38158_at | D79987 | Hs.153479 | 9700 | extra spindle poles, |
| | | | | | | | S. cerevisiae, |
| | | | | | | | homolog of |
| 181 | 0.64 | 0.562 | 910_at | M15205 | Hs.105097 | 7083 | thymidine kinase |
| | | | | | | | 1, soluble |
| 182 | 0.64 | 0.562 | 35314_at | D63880 | Hs.5719 | 9918 | chromosome |
| | | | | | | | condensation- |
| | | | | | | | related SMC- |
| | | | | | | | associated protein |
| | | | | | | | 1 |
| 183 | 0.64 | 0.561 | 41601_at | AA142964 | Hs.64311 | 6868 | a disintegrin and |
| | | | | | | | metalloproteinase |
| | | | | | | | domain 17 (tumor |
| | | | | | | | necrosis factor, |
| | | | | | | | alpha, converting |
| | | | | | | | enzyme) |
| 184 | 0.63 | 0.561 | 41824_at | AI140114 | Hs.6153 | 51096 | CGI-48 protein |
| 185 | 0.63 | 0.560 | 36184_at | L06419 | Hs.75093 | 5351 | procollagen-lysine, |
| | | | | | | | 2-oxoglutarate 5- |
| | | | | | | | dioxygenase |
| | | | | | | | (lysine |
| | | | | | | | hydroxylase, |
| | | | | | | | Ehlers-Danlos |
| | | | | | | | syndrome type VI) |
| 186 | 0.63 | 0.560 | 41133_at | U32519 | Hs.220689 | 10146 | Ras-GTPase- |
| | | | | | | | activating protein |
| | | | | | | | SH3-domain- |
| | | | | | | | binding protein |
| 187 | 0.63 | 0.559 | 35694_at | AB014587 | Hs.3628 | 9448 | mitogen-activated |
| | | | | | | | protein kinase |
| | | | | | | | kinase kinase |
| | | | | | | | kinase 4 |
| 188 | 0.63 | 0.559 | 39070_at | U03057 | Hs.118400 | 6624 | singed |
| | | | | | | | (Drosophila)-like |
| | | | | | | | (sea urchin fascin |
| | | | | | | | homolog like) |
| 189 | 0.63 | 0.559 | 1801_at | U76638 | Hs.54089 | 580 | BRCA1 associated |
| | | | | | | | RING domain 1 |
| 190 | 0.63 | 0.557 | 38405_at | U25165 | Hs.82712 | 8087 | fragile X mental |
| | | | | | | | retardation, |
| | | | | | | | autosomal |
| | | | | | | | homolog 1 |
| 191 | 0.63 | 0.557 | 38684_at | AJ010953 | Hs.106778 | 27032 | ATPase, Ca++ |
| | | | | | | | transporting, type |
| | | | | | | | 2C, member 1 |
| 192 | 0.63 | 0.554 | 31832_at | AB006624 | Hs.14912 | 23306 | KIAA0286 protein |
| 193 | 0.63 | 0.554 | 410_s_at | X57152 | Hs.165843 | 1460 | casein kinase 2, |
| | | | | | | | beta polypeptide |
| 194 | 0.62 | 0.554 | 39060_at | D38048 | Hs.118065 | 5695 | proteasome |
| | | | | | | | (prosome, |
| | | | | | | | macropain) |
| | | | | | | | subunit, beta type, |
| | | | | | | | 7 |
| 195 | 0.62 | 0.553 | 40412_at | AA203476 | Hs.252587 | 9232 | pituitary tumor- |
| | | | | | | | transforming 1 |
| 196 | 0.62 | 0.552 | 37729_at | Y08614 | Hs.79090 | 7514 | exportin 1 (CRM1, |
| | | | | | | | yeast, homolog) |
| 197 | 0.62 | 0.552 | 38863_at | L07540 | Hs.171075 | 5985 | replication factor C |
| | | | | | | | (activator 1) 5 |
| | | | | | | | (36.5 kD) |
| 198 | 0.62 | 0.551 | 37726_at | X06323 | Hs.79086 | 11222 | mitochondrial |
| | | | | | | | ribosomal protein |
| | | | | | | | L3 |
| 199 | 0.62 | 0.551 | 41003_at | U41816 | Hs.91161 | 5203 | prefoldin 4 |
| 200 | 0.62 | 0.550 | 592_at | M34079 | Hs.250758 | 5702 | proteasome |
| | | | | | | | (prosome, |
| | | | | | | | macropain) 26S |
| | | | | | | | subunit, ATPase, 3 |

According to the invention, preferred markers are markers 1-30, preferably 1-20, and more preferably 1-10.

TABLE 2
C2 Markers
Class C2

| | | | | | UNIGENE | | |
|---|---|---|---|---|---|---|---|
| | | | | | (as of | | Desc |
| | | Perm | | GB/TIGR | summer | | (unigene/locuslink |
| | s2n_obs | 0.1% | non_norm_list | Identifier | 2001) | LL_num | or affy) |
| 1 | 1.46 | 0.781 | 40035_at | AB012917 | Hs.57771 | 11012 | kallikrein 11 |
| 2 | 1.27 | 0.736 | 40544_g_at | L08424 | Hs.1619 | 429 | achaete-scute |
| | | | | | | | complex |
| | | | | | | | (Drosophila) |
| | | | | | | | homolog-like 1 |
| 3 | 1.27 | 0.721 | 36606_at | X51405 | Hs.75360 | 1363 | carboxypeptidase |
| | | | | | | | E |
| 4 | 1.21 | 0.715 | 31477_at | L08044 | Hs.82961 | 7033 | trefoil factor 3 |
| | | | | | | | (intestinal) |
| 5 | 1.18 | 0.708 | 36299_at | X02330 | | | calcitonin/ |
| | | | | | | | calcitonin- |
| | | | | | | | related |
| | | | | | | | polypeptide, |
| | | | | | | | alpha |
| 6 | 1.17 | 0.699 | 40649_at | X64810 | Hs.78977 | 5122 | proprotein |
| | | | | | | | convertase |
| | | | | | | | subtilisin/kexin |
| | | | | | | | type 1 |
| 7 | 1.16 | 0.684 | 442_at | X15187 | Hs.82689 | 7184 | tumor rejection |
| | | | | | | | antigen (gp96) 1 |
| 8 | 1.05 | 0.660 | 36300_at | X15943 | Hs.37058 | 796 | calcitonin/ |
| | | | | | | | calcitonin- |
| | | | | | | | related |
| | | | | | | | polypeptide, |
| | | | | | | | alpha |
| 9 | 1.02 | 0.658 | 39332_at | AF035316 | Hs.336780 | 7280 | tubulin, beta |
| | | | | | | | polypeptide |
| 10 | 0.97 | 0.651 | 39756_g_at | Z93930 | Hs.149923 | 7494 | X-box binding |
| | | | | | | | protein 1 |
| 11 | 0.96 | 0.647 | 39135_at | AB018310 | Hs.95180 | 23151 | KIAA0767 |
| | | | | | | | protein |
| 12 | 0.95 | 0.645 | 34785_at | AB028948 | Hs.4084 | 23389 | KIAA1025 |
| | | | | | | | protein |
| 13 | 0.92 | 0.644 | 37617_at | U90912 | Hs.81897 | 54462 | KIAA1128 |
| | | | | | | | protein |
| 14 | 0.85 | 0.630 | 1788_s_at | U48807 | Hs.2359 | 1846 | dual specificity |
| | | | | | | | phosphatase 4 |
| 15 | 0.85 | 0.630 | 37928_at | AA621555 | Hs.84928 | 4801 | nuclear |
| | | | | | | | transcription |
| | | | | | | | factor Y, beta |
| 16 | 0.84 | 0.625 | 37141_at | U39840 | Hs.299867 | 3169 | hepatocyte |
| | | | | | | | nuclear factor 3, |
| | | | | | | | alpha |
| 17 | 0.84 | 0.623 | 35995 at | AF067656 | Hs.42650 | 11130 | ZW10 interactor |
| 18 | 0.83 | 0.622 | 40201_at | M76180 | Hs.150403 | 1644 | dopa |
| | | | | | | | decarboxylase |
| | | | | | | | (aromatic L- |
| | | | | | | | amino acid |
| | | | | | | | decarboxylase) |
| 19 | 0.82 | 0.620 | 35800_at | D63391 | Hs.6793 | 5050 | platelet- |
| | | | | | | | activating factor |
| | | | | | | | acetylhydrolase, |
| | | | | | | | isoform Ib, |
| | | | | | | | gamma subunit |
| | | | | | | | (29 kD) |
| 20 | 0.8 | 0.618 | 33543_s_at | U77718 | Hs.44499 | 5411 | pinin, |
| | | | | | | | desmosome |
| | | | | | | | associated |
| | | | | | | | protein |
| 21 | 0.8 | 0.615 | 1822_at | HG4677- | | | Oncogene |
| | | | | HT5102 | | | Ret/Ptc2, Fusion |
| | | | | | | | Activated |
| 22 | 0.79 | 0.613 | 35343_at | M37400 | Hs.597 | 2805 | glutamic- |
| | | | | | | | oxaloacetic |
| | | | | | | | transaminase 1, |
| | | | | | | | soluble |
| | | | | | | | (aspartate |
| | | | | | | | aminotransferase |
| | | | | | | | 1) |
| 23 | 0.78 | 0.610 | 41403_at | AI032612 | Hs.105465 | 6636 | small nuclear |
| | | | | | | | ribonucleoprotein |
| | | | | | | | polypeptide F |
| 24 | 0.78 | 0.606 | 37426_at | U80736 | Hs.110826 | 27324 | trinucleotide |
| | | | | | | | repeat |
| | | | | | | | containing 9 |
| 25 | 0.77 | 0.605 | 39113_at | AI262789 | Hs.93659 | 9601 | protein disulfide |
| | | | | | | | isomerase |
| | | | | | | | related protein |
| | | | | | | | (calcium- |
| | | | | | | | binding protein, |
| | | | | | | | intestinal- |
| | | | | | | | related) |
| 26 | 0.77 | 0.604 | 40881_at | X64330 | Hs.174140 | 47 | ATP citrate |
| | | | | | | | lyase |
| 27 | 0.77 | 0.603 | 32137_at | AF029778 | Hs.166154 | 3714 | jagged 2 |
| 28 | 0.77 | 0.600 | 34690_at | U66616 | Hs.236030 | 6601 | SWI/SNF |
| | | | | | | | related, matrix |
| | | | | | | | associated, actin |
| | | | | | | | dependent |
| | | | | | | | regulator of |
| | | | | | | | chromatin, |
| | | | | | | | subfamily c, |
| | | | | | | | member 2 |
| 29 | 0.77 | 0.599 | 41395_at | AB003791 | Hs.104576 | 8534 | carbohydrate |
| | | | | | | | (keratan sulfate |
| | | | | | | | Gal-6) |
| | | | | | | | sulfotransferase |
| | | | | | | | 1 |
| 30 | 0.76 | 0.599 | 39891_at | AI246730 | Hs.126901 | | cDNA, 3 end |
| 31 | 0.76 | 0.598 | 41250_at | U24169 | Hs.301613 | 7965 | JTV1 gene |
| 32 | 0.76 | 0.598 | 37545_at | W22110 | Hs.7934 | 9314 | Kruppel-like |
| | | | | | | | factor 4 (gut) |
| 33 | 0.75 | 0.597 | 41146_at | J03473 | Hs.177766 | 142 | ADP- |
| | | | | | | | ribosyltransferase |
| | | | | | | | (NAD+; poly |
| | | | | | | | (ADP-ribose) |
| | | | | | | | polymerase) |
| 34 | 0.74 | 0.597 | 40865_at | U51166 | Hs.173824 | 6996 | thymine-DNA |
| | | | | | | | glycosylase |
| 35 | 0.74 | 0.597 | 35147_at | AB002360 | Hs.25515 | 23263 | MCF.2 cell line |
| | | | | | | | derived |
| | | | | | | | transforming |
| | | | | | | | sequence-like |
| 36 | 0.74 | 0.591 | 36847_r_at | AA121509 | Hs.70830 | 51690 | U6 snRNA- |
| | | | | | | | associated Sm- |
| | | | | | | | like protein |
| | | | | | | | LSm7 |
| 37 | 0.73 | 0.588 | 37293_at | D43948 | Hs.76989 | 9793 | KIAA0097 gene |
| | | | | | | | product |
| 38 | 0.73 | 0.587 | 36482_s_at | Y15724 | Hs.5541 | 489 | ATPase, Ca++ |
| | | | | | | | transporting, |
| | | | | | | | ubiquitous |
| 39 | 0.72 | 0.586 | 38654_at | X65488 | Hs.103804 | 3192 | heterogeneous |
| | | | | | | | nuclear |
| | | | | | | | ribonucleoprotein |
| | | | | | | | U (scaffold |
| | | | | | | | attachment |
| | | | | | | | factor A) |
| 40 | 0.72 | 0.583 | 37359_at | D14658 | Hs.77665 | 9789 | KIAA0102 gene |
| | | | | | | | product |
| 41 | 0.72 | 0.582 | 37638_at | D50857 | Hs.82295 | 1793 | dedicator of |
| | | | | | | | cyto-kinesis 1 |
| 42 | 0.72 | 0.582 | 39824_at | AI391564 | Hs.110820 | | cDNA, 3 end |
| 43 | 0.71 | 0.580 | 37019_at | J00129 | Hs.7645 | 2244 | fibrinogen, B |
| | | | | | | | beta polypeptide |
| 44 | 0.71 | 0.578 | 40074_at | X16396 | Hs.154672 | 10797 | methylene |
| | | | | | | | tetrahydrofolate |
| | | | | | | | dehydrogenase |
| | | | | | | | (NAD+ |
| | | | | | | | dependent), |
| | | | | | | | methenyltetra- |
| | | | | | | | hydrofolate |
| | | | | | | | cyclohydrolase |
| 45 | 0.71 | 0.576 | 40584_at | Y08612 | Hs.172108 | 4927 | nucleoporin |
| | | | | | | | 88 kD |
| 46 | 0.7 | 0.576 | 33266_at | AF015254 | Hs.180655 | 9212 | serine/threonine |
| | | | | | | | kinase 12 |
| 47 | 0.69 | 0.575 | 36008_at | AF041434 | Hs.43666 | 11156 | protein tyrosine |
| | | | | | | | phosphatase |
| | | | | | | | type IVA, |
| | | | | | | | member 3 |
| 48 | 0.69 | 0.574 | 37333_at | X63692 | Hs.77462 | 1786 | DNA (cytosine- |
| | | | | | | | 5-)- |
| | | | | | | | methyltransferase |
| | | | | | | | 1 |
| 49 | 0.69 | 0.574 | 1660_at | D83004 | Hs.75355 | 7334 | ubiquitin- |
| | | | | | | | conjugating |
| | | | | | | | enzyme E2N |
| | | | | | | | (homologous to |
| | | | | | | | yeast UBC13) |
| 50 | 0.69 | 0.573 | 36149_at | D78014 | Hs.74566 | 1809 | dihydro- |
| | | | | | | | pyrimidinase- |
| | | | | | | | like 3 |
| 51 | 0.68 | 0.573 | 39692_at | AL080209 | Hs.13659 | 64764 | hypothetical |
| | | | | | | | protein |
| | | | | | | | DKFZp586F2423 |
| 52 | 0.68 | 0.570 | 40317_at | U57352 | Hs.6517 | 40 | amiloride- |
| | | | | | | | sensitive cation |
| | | | | | | | channel 1, |
| | | | | | | | neuronal |
| | | | | | | | (degenerin) |
| 53 | 0.67 | 0.568 | 31906_at | AF068754 | Hs.250899 | 3281 | heat shock |
| | | | | | | | factor binding |
| | | | | | | | protein 1 |
| 54 | 0.67 | 0.567 | 149_at | U90426 | Hs.179606 | 10212 | nuclear RNA |
| | | | | | | | helicase, DECD |
| | | | | | | | variant of |
| | | | | | | | DEAD box |
| | | | | | | | family |
| 55 | 0.67 | 0.567 | 38978_at | AF013758 | Hs.109643 | 10605 | polyadenylate |
| | | | | | | | binding protein- |
| | | | | | | | interacting |
| | | | | | | | protein 1 |
| 56 | 0.67 | 0.565 | 35566_f_at | AF015128 | Hs.301365 | | IgG heavy chain |
| | | | | | | | variable region |
| | | | | | | | (Vh26) |
| 57 | 0.66 | 0.564 | 36745_at | AF035308 | Hs.167036 | | clone 23798 and |
| | | | | | | | 23825 |
| 58 | 0.66 | 0.563 | 36133_at | AL031058 | Hs.74316 | 1832 | desmoplakin |
| | | | | | | | (DPI, DPII) |
| 59 | 0.66 | 0.563 | 35966_at | X71125 | Hs.79033 | 25797 | glutaminyl- |
| | | | | | | | peptide |
| | | | | | | | cyclotransferase |
| | | | | | | | (glutaminyl |
| | | | | | | | cyclase) |
| 60 | 0.66 | 0.562 | 37955_at | AB015631 | Hs.8752 | 10330 | transmembrane |
| | | | | | | | protein 4 |
| 61 | 0.65 | 0.562 | 40846_g_at | U10324 | Hs.256583 | 3609 | interleukin |
| | | | | | | | enhancer |
| | | | | | | | binding factor 3, |
| | | | | | | | 90 kD |
| 62 | 0.65 | 0.560 | 37101_at | AL050008 | Hs.306186 | 25855 | DKFZP564A063 |
| | | | | | | | protein |
| 63 | 0.65 | 0.559 | 40580_r_at | M24398 | Hs.171814 | 5763 | parathymosin |
| 64 | 0.65 | 0.559 | 36489_at | D00860 | Hs.56 | 5631 | phosphoribosyl |
| | | | | | | | pyrophosphate |
| | | | | | | | synthetase 1 |
| 65 | 0.65 | 0.558 | 37133_at | AF027406 | Hs.104865 | 26576 | serine/threonine |
| | | | | | | | kinase 23 |
| 66 | 0.64 | 0.557 | 33714_at | Y10043 | Hs.19114 | 3149 | high-mobility |
| | | | | | | | group |
| | | | | | | | (nonhistone |
| | | | | | | | chromosomal) |
| | | | | | | | protein 4 |
| 67 | 0.64 | 0.557 | 35351_at | U89505 | Hs.6106 | 5936 | RNA binding |
| | | | | | | | motif protein 4 |
| 68 | 0.64 | 0.557 | 41829_at | AB018274 | Hs.6214 | 23367 | KIAA0731 |
| | | | | | | | protein |
| 69 | 0.64 | 0.555 | 39158_at | AB021663 | Hs.9754 | 22809 | activating |
| | | | | | | | transcription |
| | | | | | | | factor 5 |
| 70 | 0.64 | 0.555 | 35163_at | AB028964 | Hs.26023 | 22887 | KIAA1041 |
| | | | | | | | protein |
| 71 | 0.64 | 0.555 | 36406_at | AA401397 | Hs.165296 | 26085 | kallikrein 13 |
| 72 | 0.63 | 0.554 | 32149_at | AA532495 | Hs.183752 | 4477 | microsemino- |
| | | | | | | | protein, beta- |
| 73 | 0.63 | 0.554 | 32825_at | Y10805 | Hs.20521 | 3276 | HMT1 (hnRNP |
| | | | | | | | methyltransferase, |
| | | | | | | | S. cerevisiae)- |
| | | | | | | | like 2 |
| 74 | 0.63 | 0.553 | 35590_s_at | X81832 | | | gastric |
| | | | | | | | inhibitory |
| | | | | | | | polypeptide |
| | | | | | | | receptor |
| 75 | 0.63 | 0.553 | 36636_at | M12267 | Hs.75485 | 4942 | ornithine |
| | | | | | | | aminotransferase |
| | | | | | | | (gyrate |
| | | | | | | | atrophy) |
| 76 | 0.63 | 0.553 | 37944_at | U19523 | Hs.86724 | 2643 | GTP |
| | | | | | | | cyclohydrolase |
| | | | | | | | 1 (dopa- |
| | | | | | | | responsive |
| | | | | | | | dystonia) |
| 77 | 0.63 | 0.552 | 41083_at | AC006276 | Hs.99093 | | chromosome 19, |
| | | | | | | | cosmid R28379 |
| 78 | 0.62 | 0.550 | 39317_at | D86324 | Hs.24697 | 8418 | cytidine |
| | | | | | | | monophosphate- |
| | | | | | | | N- |
| | | | | | | | acetylneuraminic |
| | | | | | | | acid |
| | | | | | | | hydroxylase |
| | | | | | | | (CMP-N- |
| | | | | | | | acetylneuraminate |
| | | | | | | | monooxygenase) |
| 79 | 0.62 | 0.550 | 33162_at | X02160 | Hs.89695 | 3643 | insulin receptor |
| 80 | 0.62 | 0.549 | 31586_f_at | X72475 | Hs.156110 | 3514 | immunoglobulin |
| | | | | | | | kappa constant |
| 81 | 0.62 | 0.549 | 34289_f_at | D50920 | Hs.23106 | 9862 | KIAA0130 gene |
| | | | | | | | product |
| 82 | 0.62 | 0.549 | 36615_at | M83751 | Hs.75412 | 7873 | Arginine-rich |
| | | | | | | | protein |
| 83 | 0.62 | 0.546 | 904_s_at | L47276 | | | (cell line HL- |
| | | | | | | | 60) alpha |
| | | | | | | | topoisomerase |
| | | | | | | | truncated-form |
| | | | | | | | mRNA, 3 UTR |
| 84 | 0.62 | 0.545 | 39791_at | M23114 | Hs.1526 | 488 | ATPase, Ca++ |
| | | | | | | | transporting, |
| | | | | | | | cardiac muscle, |
| | | | | | | | slow twitch 2 |
| 85 | 0.62 | 0.544 | 36203_at | X16277 | Hs.75212 | 4953 | ornithine |
| | | | | | | | decarboxylase 1 |
| 86 | 0.61 | 0.544 | 1582_at | M29540 | Hs.220529 | 1048 | carcinoembryonic |
| | | | | | | | antigen- |
| | | | | | | | related cell |
| | | | | | | | adhesion |
| | | | | | | | molecule 5 |
| 87 | 0.61 | 0.544 | 38456_s_at | AL049650 | Hs.83753 | 6628 | small nuclear |
| | | | | | | | ribonucleoprotein |
| | | | | | | | polypeptides |
| | | | | | | | B and B1 |
| 88 | 0.61 | 0.544 | 39610_at | X16665 | Hs.2733 | 3212 | homeo box B2 |
| 89 | 0.61 | 0.544 | 37272_at | X57206 | Hs.78877 | 3707 | inositol 1,4,5- |
| | | | | | | | trisphosphate 3- |
| | | | | | | | kinase B |
| 90 | 0.61 | 0.544 | 36185_at | D32050 | Hs.75102 | 16 | alanyl-tRNA |
| | | | | | | | synthetase |
| 91 | 0.61 | 0.544 | 38435_at | U25182 | Hs.83383 | 10549 | thioredoxin |
| | | | | | | | peroxidase |
| | | | | | | | (antioxidant |
| | | | | | | | enzyme) |
| 92 | 0.6 | 0.544 | 32447_at | U76388 | Hs.157037 | 2516 | nuclear receptor |
| | | | | | | | subfamily 5, |
| | | | | | | | group A, |
| | | | | | | | member 1 |
| 93 | 0.6 | 0.544 | 38753_at | AF039022 | Hs.85951 | 11260 | exportin, tRNA |
| | | | | | | | (nuclear export |
| | | | | | | | receptor for |
| | | | | | | | tRNAs) |
| 94 | 0.6 | 0.543 | 38248_at | AB011124 | Hs.90232 | 9762 | KIAA0552 gene |
| | | | | | | | product |
| 95 | 0.6 | 0.543 | 38719_at | U03985 | Hs.108802 | 4905 | N- |
| | | | | | | | ethylmaleimide- |
| | | | | | | | sensitive factor |
| 96 | 0.6 | 0.543 | 34105_f_at | AI147237 | Hs.300697 | 3502 | immunoglobulin |
| | | | | | | | heavy constant |
| | | | | | | | gamma 3 (G3m |
| | | | | | | | marker) |
| 97 | 0.6 | 0.543 | 40840_at | M80254 | Hs.173125 | 10105 | peptidylprolyl |
| | | | | | | | isomerase F |
| | | | | | | | (cyclophilin F) |
| 98 | 0.6 | 0.542 | 1745_at | HG4679- | | | Oncogene |
| | | | | HT5104 | | | Ret/Ptc, Fusion |
| | | | | | | | Activated |
| 99 | 0.59 | 0.542 | 1884_s_at | M15796 | Hs.78996 | 5111 | proliferating |
| | | | | | | | cell nuclear |
| | | | | | | | antigen |
| 100 | 0.59 | 0.542 | 31935_s_at | U75968 | Hs.27424 | 1663 | DEAD/H (Asp- |
| | | | | | | | Glu-Ala- |
| | | | | | | | Asp/His) box |
| | | | | | | | polypeptide 11 |
| | | | | | | | (S. cerevisiae |
| | | | | | | | CHL1-like |
| | | | | | | | helicase) |
| 101 | 0.59 | 0.542 | 34933_at | AJ238381 | Hs.132576 | 5083 | paired box gene |
| | | | | | | | 9 |
| 102 | 0.59 | 0.542 | 33304_at | U88964 | Hs.183487 | 3669 | interferon |
| | | | | | | | stimulated gene |
| | | | | | | | (20 kD) |
| 103 | 0.59 | 0.542 | 38340_at | AB014555 | Hs.96731 | 9026 | huntingtin |
| | | | | | | | interacting |
| | | | | | | | protein- 1- |
| | | | | | | | related |
| 104 | 0.58 | 0.542 | 1796_s_at | U05681 | | | B-cell |
| | | | | | | | CLL/lymphoma |
| | | | | | | | 3 |
| 105 | 0.58 | 0.542 | 34726_at | U07139 | Hs.250712 | 784 | calcium |
| | | | | | | | channel, |
| | | | | | | | voltage- |
| | | | | | | | dependent, beta |
| | | | | | | | 3 subunit |
| 106 | 0.58 | 0.541 | 35253_at | AB011143 | Hs.30687 | 9846 | GRB2- |
| | | | | | | | associated |
| | | | | | | | binding protein |
| | | | | | | | 2 |
| 107 | 0.58 | 0.541 | 35151_at | AF089814 | Hs.25664 | 10263 | tumor |
| | | | | | | | suppressor |
| | | | | | | | deleted in oral |
| | | | | | | | cancer-related 1 |
| 108 | 0.58 | 0.541 | 38635_at | Z69043 | Hs.102135 | 6748 | signal sequence |
| | | | | | | | receptor, delta |
| | | | | | | | (translocon- |
| | | | | | | | associated |
| | | | | | | | protein delta) |
| 109 | 0.58 | 0.541 | 39040_at | W28360 | Hs.184325 | 51632 | CGI-76 protein |
| 110 | 0.57 | 0.541 | 38860_at | U66346 | Hs.189 | 5143 | phosphodiesterase |
| | | | | | | | 4C, cAMP- |
| | | | | | | | specific (dunce |
| | | | | | | | (Drosophila)- |
| | | | | | | | homolog |
| | | | | | | | phosphodiesterase |
| | | | | | | | E1) |
| 111 | 0.57 | 0.541 | 1432_s_at | D16105 | Hs.210 | 4058 | leukocyte |
| | | | | | | | tyrosine kinase |
| 112 | 0.57 | 0.541 | 36851_g_at | U42360 | | | Putative |
| | | | | | | | prostate cancer |
| | | | | | | | tumor |
| | | | | | | | suppressor |
| 113 | 0.57 | 0.540 | 37985_at | L37747 | | | lamin B1 |
| 114 | 0.57 | 0.540 | 38708_at | AF054183 | Hs.10842 | 5901 | RAN, member |
| | | | | | | | RAS oncogene |
| | | | | | | | family |
| 115 | 0.57 | 0.540 | 32404_at | AF065314 | Hs.234785 | 1261 | cyclic |
| | | | | | | | nucleotide gated |
| | | | | | | | channel alpha 3 |
| 116 | 0.57 | 0.540 | 36970_at | D80004 | Hs.75909 | 23199 | KIAA0182 |
| | | | | | | | protein |
| 117 | 0.57 | 0.540 | 32646_at | AB007918 | Hs.169182 | 23046 | KIAA0449 |
| | | | | | | | protein |
| 118 | 0.57 | 0.539 | 32485_at | X00371 | Hs.118836 | 4151 | myoglobin |
| 119 | 0.57 | 0.538 | 37774_at | AI819942 | Hs.90998 | 23157 | septin 2 |
| 120 | 0.57 | 0.538 | 36153_at | L13848 | Hs.74578 | 1660 | DEAD/H (Asp- |
| | | | | | | | Glu-Ala- |
| | | | | | | | Asp/His) box |
| | | | | | | | polypeptide 9 |
| | | | | | | | (RNA helicase |
| | | | | | | | A, nuclear DNA |
| | | | | | | | helicase II; |
| | | | | | | | leukophysin) |
| 121 | 0.57 | 0.538 | 288_s_at | L25931 | Hs.152931 | 3930 | lamin B |
| | | | | | | | receptor |
| 122 | 0.56 | 0.538 | 33347_at | AA883868 | Hs.216354 | 6048 | ring finger |
| | | | | | | | protein 5 |
| 123 | 0.56 | 0.538 | 33399_at | AA142942 | Hs.241507 | 6194 | ribosomal |
| | | | | | | | protein S6 |
| 124 | 0.56 | 0.538 | 1888_s_at | X06182 | Hs.81665 | 3815 | v-kit Hardy- |
| | | | | | | | Zuckerman 4 |
| | | | | | | | feline sarcoma |
| | | | | | | | viral oncogene |
| | | | | | | | homolog |
| 125 | 0.56 | 0.538 | 1846_at | L78132 | Hs.4082 | 3964 | prostate |
| | | | | | | | carcinoma |
| | | | | | | | tumor antigen |
| | | | | | | | (pcta-1)/lectin |
| 126 | 0.56 | 0.537 | 34338_at | D49738 | Hs.31053 | 1155 | cytoskeleton- |
| | | | | | | | associated |
| | | | | | | | protein 1 |
| 127 | 0.56 | 0.537 | 41241_at | D84273 | Hs.181311 | 4677 | asparaginyl- |
| | | | | | | | tRNA |
| | | | | | | | synthetase |
| 128 | 0.56 | 0.536 | 35670_at | M37457 | | | ATPase, |
| | | | | | | | Na+/K+ |
| | | | | | | | transporting, |
| | | | | | | | alpha 3 |
| | | | | | | | polypeptide |
| 129 | 0.56 | 0.536 | 41399_at | AB029034 | Hs.285641 | 23133 | KIAA1111 |
| | | | | | | | protein |
| 130 | 0.55 | 0.536 | 36676_at | AL031659 | Hs.75722 | 6185 | growth hormone |
| | | | | | | | releasing |
| | | | | | | | hormone |
| 131 | 0.55 | 0.536 | 39927_at | U17032 | Hs.267831 | 394 | Rho GTPase |
| | | | | | | | activating |
| | | | | | | | protein 5 |
| 132 | 0.55 | 0.536 | 1257_s_at | L42379 | Hs.77266 | 5768 | quiescin Q6 |
| 133 | 0.55 | 0.535 | 37576_at | U52969 | Hs.80296 | 5121 | Purkinje cell |
| | | | | | | | protein 4 |
| 134 | 0.55 | 0.535 | 34987_s_at | X79536 | Hs.249495 | 3178 | heterogeneous |
| | | | | | | | nuclear |
| | | | | | | | ribonucleoprotein |
| | | | | | | | A1 |
| 135 | 0.55 | 0.535 | 1798_at | U41060 | Hs.79136 | 25800 | LIV-1 protein, |
| | | | | | | | estrogen |
| | | | | | | | regulated |
| 136 | 0.55 | 0.535 | 40674_s_at | S82986 | Hs.820 | 3223 | homeo box C6 |
| 137 | 0.55 | 0.535 | 39342_at | X94754 | Hs.279946 | 4141 | methionine- |
| | | | | | | | tRNA |
| | | | | | | | synthetase |
| 138 | 0.55 | 0.535 | 38707_r_at | S75174 | Hs.108371 | 1874 | E2F |
| | | | | | | | transcription |
| | | | | | | | factor 4, |
| | | | | | | | p107/p130- |
| | | | | | | | binding |
| 139 | 0.55 | 0.535 | 34648_at | Z12830 | Hs.250773 | 6745 | signal sequence |
| | | | | | | | receptor, alpha |
| | | | | | | | (translocon- |
| | | | | | | | associated |
| | | | | | | | protein alpha) |
| 140 | 0.54 | 0.535 | 40653_at | U32439 | Hs.79348 | 6000 | regulator of G- |
| | | | | | | | protein |
| | | | | | | | signalling 7 |
| 141 | 0.54 | 0.534 | 34827_at | AF045458 | Hs.47061 | 8408 | unc-51 (C. |
| | | | | | | | elegans)-like |
| | | | | | | | kinase 1 |
| 142 | 0.54 | 0.534 | 36178_at | U23143 | Hs.75069 | 6472 | serine |
| | | | | | | | hydroxymethyl- |
| | | | | | | | transferase 2 |
| | | | | | | | (mitochondrial) |
| 143 | 0.54 | 0.534 | 34264_at | AB026894 | Hs.226499 | 23623 | nesca protein |
| 144 | 0.54 | 0.534 | 41750_at | D49489 | Hs.182429 | 10130 | protein disulfide |
| | | | | | | | isomerase- |
| | | | | | | | related protein |
| 145 | 0.54 | 0.534 | 36971_at | D87446 | Hs.75912 | 23505 | KIAA0257 |
| | | | | | | | protein |
| 146 | 0.54 | 0.534 | 38399_at | AL034428 | Hs.82575 | 6629 | small nuclear |
| | | | | | | | ribonucleoprotein |
| | | | | | | | polypeptide |
| | | | | | | | B″ |
| 147 | 0.54 | 0.534 | 32190_at | AL050118 | Hs.184641 | 9415 | fatty acid |
| | | | | | | | desaturase 2 |
| 148 | 0.54 | 0.534 | 38835_at | U94831 | Hs.91586 | 10548 | transmembrane |
| | | | | | | | 9 superfamily |
| | | | | | | | member 1 |
| 149 | 0.54 | 0.533 | 37316_r_at | AI057607 | Hs.7731 | 55837 | uncharacterized |
| | | | | | | | bone marrow |
| | | | | | | | protein BM036 |

The C2 class is a robust class of markers. According to the invention, preferred markers are markers 1-30, preferably 1-20, and more preferably 1-10. Highly preferred markers are kallikrein 11, achaete-scute complex (Drosophila) homolog-like 1, carboxypeptidase E, trefoil factor 3 (intestinal), calcitonin/calcitonin-related polypeptide alpha, proprotein convertase, dual specificity phosphatase 4, and dopa decarboxylase.

TABLE 3
C3 Markers
Class C3

| | | | | | UNIGENE | | |
|---|---|---|---|---|---|---|---|
| | | | | | (as of | | Desc |
| | s2n_o | Perm | | GB/TIGR | summer | | (unigene/locuslink |
| | bs | 0.1% | non_norm_list | Identifier | 2001) | LL_num | or affy) |
| 1 | 1.42 | 0.866 | 37669_s_at | U16799 | Hs.78629 | 481 | ATPase, Na+/K+ |
| | | | | | | | transporting, beta 1 |
| | | | | | | | polypeptide |
| 2 | 1.2 | 0.724 | 36066_at | AB020635 | Hs.4984 | 23382 | KIAA0828 protein |
| 3 | 1.17 | 0.707 | 33699_at | M18667 | | | progastricsin |
| | | | | | | | (pepsinogen C) |
| 4 | 1.06 | 0.706 | 1081_at | M33764 | Hs.75212 | 4953 | ornithine |
| | | | | | | | decarboxylase 1 |
| 5 | 1.06 | 0.688 | 33396_at | U12472 | Hs.226795 | 2950 | glutathione S- |
| | | | | | | | transferase pi |
| 6 | 1.06 | 0.679 | 34319_at | AA131149 | Hs.2962 | 6286 | S100 calcium- |
| | | | | | | | binding protein P |
| 7 | 1.02 | 0.674 | 40409_at | U46689 | Hs.159608 | 224 | aldehyde |
| | | | | | | | dehydrogenase 10 |
| | | | | | | | (fatty aldehyde |
| | | | | | | | dehydrogenase) |
| 8 | 1.02 | 0.673 | 32805_at | U05861 | | | aldo-keto reductase |
| | | | | | | | family 1, member |
| | | | | | | | C1 (dihydrodiol |
| | | | | | | | dehydrogenase 1; |
| | | | | | | | 20-alpha (3-alpha)- |
| | | | | | | | hydroxysteroid |
| | | | | | | | dehydrogenase) |
| 9 | 0.99 | 0.667 | 33383_f_at | AI820718 | Hs.250505 | 5914 | retinoic acid |
| | | | | | | | receptor, alpha |
| 10 | 0.98 | 0.663 | 35207_at | X76180 | Hs.2794 | 6337 | sodium channel, |
| | | | | | | | nonvoltage-gated 1 |
| | | | | | | | alpha |
| 11 | 0.98 | 0.655 | 33052_at | U95301 | Hs.144442 | 8399 | phospholipase A2, |
| | | | | | | | group X |
| 12 | 0.98 | 0.649 | 38526_at | U02882 | Hs.172081 | 5144 | phosphodiesterase |
| | | | | | | | 4D, cAMP-specific |
| | | | | | | | (dunce |
| | | | | | | | (Drosophila)- |
| | | | | | | | homolog |
| | | | | | | | phosphodiesterase |
| | | | | | | | E3) |
| 13 | 0.97 | 0.646 | 38066_at | M81600 | | | diaphorase |
| | | | | | | | (NADH/NADPH) |
| | | | | | | | (cytochrome b-5 |
| | | | | | | | reductase) |
| 14 | 0.93 | 0.644 | 1882_g_at | HG4058- | | | Oncogene Aml1- |
| | | | | HT4328 | | | Evi-1, Fusion |
| | | | | | | | Activated |
| 15 | 0.93 | 0.643 | 37779_at | Y08134 | Hs.123659 | 27293 | acid |
| | | | | | | | sphingomyelinase- |
| | | | | | | | like |
| | | | | | | | phosphodiesterase |
| 16 | 0.92 | 0.641 | 38773_at | AB003151 | Hs.88778 | 873 | carbonyl reductase |
| | | | | | | | 1 |
| 17 | 0.9 | 0.639 | 700_s_at | HG371- | | | Mucin 1, |
| | | | | HT26388 | | | Epithelial, Alt. |
| | | | | | | | Splice 9 |
| 18 | 0.89 | 0.639 | 37004_at | J02761 | Hs.76305 | 6439 | surfactant, |
| | | | | | | | pulmonary- |
| | | | | | | | associated protein B |
| 19 | 0.88 | 0.639 | 38986_at | Z49835 | Hs.289101 | 2923 | glucose regulated |
| | | | | | | | protein, 58 kD |
| 20 | 0.88 | 0.638 | 40685_at | U10868 | Hs.83155 | 221 | aldehyde |
| | | | | | | | dehydrogenase 7 |
| 21 | 0.87 | 0.636 | 35938_at | M72393 | Hs.211587 | 5321 | phospholipase A2, |
| | | | | | | | group IV A |
| | | | | | | | (cytosolic, calcium- |
| | | | | | | | dependent) |
| 22 | 0.87 | 0.632 | 41267_at | AB028972 | Hs.227835 | 22980 | KIAA1049 protein |
| 23 | 0.86 | 0.628 | 34839_at | AB029027 | Hs.279039 | 22910 | KIAA1104 protein |
| 24 | 0.85 | 0.627 | 38784_g_at | J05581 | Hs.89603 | 4582 | mucin 1, |
| | | | | | | | transmembrane |
| 25 | 0.83 | 0.627 | 33439_at | D15050 | Hs.232068 | 6935 | transcription factor |
| | | | | | | | 8 (represses |
| | | | | | | | interleukin 2 |
| | | | | | | | expression) |
| 26 | 0.82 | 0.627 | 38429_at | U29344 | Hs.83190 | 2194 | fatty acid synthase |
| 27 | 0.82 | 0.626 | 39248_at | N74607 | Hs.234642 | 360 | aquaporin 3 |
| 28 | 0.8 | 0.625 | 1563_s_at | M58286 | Hs.159 | 7132 | tumor necrosis |
| | | | | | | | factor receptor |
| | | | | | | | superfamily, |
| | | | | | | | member 1A |
| 29 | 0.8 | 0.623 | 39260_at | U59185 | Hs.23590 | 9122 | solute carrier family |
| | | | | | | | 16 (monocarboxylic |
| | | | | | | | acid transporters), |
| | | | | | | | member 4 |
| 30 | 0.79 | 0.623 | 38801_at | AI742846 | Hs.9006 | 9218 | VAMP (vesicle- |
| | | | | | | | associated |
| | | | | | | | membrane protein)- |
| | | | | | | | associated protein A |
| | | | | | | | (33 kD) |
| 31 | 0.79 | 0.622 | 37311_at | AF010400 | | | transaldolase 1 |
| 32 | 0.78 | 0.622 | 36200_at | X69838 | Hs.75196 | 10919 | ankyrin repeat- |
| | | | | | | | containing protein |
| 33 | 0.78 | 0.620 | 36938_at | U70063 | Hs.75811 | 427 | N-acylsphingosine |
| | | | | | | | amidohydrolase |
| | | | | | | | (acid ceramidase) |
| 34 | 0.77 | 0.618 | 41051_at | X95073 | Hs.96247 | 7257 | translin-associated |
| | | | | | | | factor X |
| 35 | 0.77 | 0.618 | 32072_at | U40434 | Hs.155981 | 10232 | mesothelin |
| 36 | 0.76 | 0.618 | 41402_at | AL080121 | Hs.105460 | 25849 | DKFZP564O0823 |
| | | | | | | | protein |
| 37 | 0.76 | 0.617 | 39392_at | AJ002190 | Hs.12482 | 8443 | glyceronephosphate |
| | | | | | | | O-acyltransferase |
| 38 | 0.75 | 0.617 | 1346_at | S72043 | Hs.73133 | 4504 | metallothionein 3 |
| | | | | | | | (growth inhibitory |
| | | | | | | | factor |
| | | | | | | | (neurotrophic)) |
| 39 | 0.74 | 0.617 | 34798_at | Z35491 | Hs.41714 | 573 | BCL2-associated |
| | | | | | | | athanogene |
| 40 | 0.72 | 0.616 | 35151_at | AF089814 | Hs.25664 | 10263 | tumor suppressor |
| | | | | | | | deleted in oral |
| | | | | | | | cancer-related 1 |
| 41 | 0.72 | 0.616 | 41772_at | M68840 | Hs.183109 | 4128 | monoamine oxidase |
| | | | | | | | A |
| 42 | 0.72 | 0.613 | 40223_r_at | AI677689 | Hs.296406 | 9701 | KIAA0685 gene |
| | | | | | | | product |
| 43 | 0.71 | 0.612 | 37399_at | D17793 | Hs.78183 | 8644 | aldo-keto reductase |
| | | | | | | | family 1, member |
| | | | | | | | C3 (3-alpha |
| | | | | | | | hydroxysteroid |
| | | | | | | | dehydrogenase, |
| | | | | | | | type II) |
| 44 | 0.71 | 0.611 | 37748_at | D86985 | Hs.79276 | 9778 | KIAA0232 gene |
| | | | | | | | product |
| 45 | 0.7 | 0.610 | 39689_at | AI362017 | Hs.135084 | 1471 | cystatin C (amyloid |
| | | | | | | | angiopathy and |
| | | | | | | | cerebral |
| | | | | | | | hemorrhage) |
| 46 | 0.7 | 0.610 | 38827_at | AF038451 | Hs.91011 | 10551 | anterior gradient 2 |
| | | | | | | | (Xenepus laevis) |
| | | | | | | | homolog |
| 47 | 0.7 | 0.609 | 36945_at | X94910 | Hs.75841 | 10961 | endoplasmic |
| | | | | | | | reticulum lumenal |
| | | | | | | | protein |
| 48 | 0.7 | 0.608 | 1662_r_at | HG2261- | | | Antigen, Prostate |
| | | | | HT2351 | | | Specific, Alt. Splice |
| | | | | | | | Form 2 |
| 49 | 0.69 | 0.608 | 38482_at | AJ011497 | Hs.278562 | 1366 | claudin 7 |
| 50 | 0.68 | 0.606 | 33325_at | W26667 | Hs.184581 | | cDNA |
| 51 | 0.68 | 0.606 | 35311_at | AF084523 | Hs.5710 | 8804 | cellular repressor of |
| | | | | | | | E1A-stimulated |
| | | | | | | | genes |
| 52 | 0.67 | 0.604 | 38063_at | U00952 | Hs.8068 | 57326 | hematopoietic |
| | | | | | | | PBX-interacting |
| | | | | | | | protein |
| 53 | 0.67 | 0.604 | 33863_at | U65785 | Hs.277704 | 10525 | oxygen regulated |
| | | | | | | | protein (150 kD) |
| 54 | 0.66 | 0.604 | 38790_at | L25879 | Hs.89649 | 2052 | epoxide hydrolase |
| | | | | | | | 1, microsomal |
| | | | | | | | (xenobiotic) |
| 55 | 0.66 | 0.602 | 35214_at | AF061016 | Hs.28309 | 7358 | UDP-glucose |
| | | | | | | | dehydrogenase |
| 56 | 0.66 | 0.602 | 37279_at | U10550 | Hs.79022 | 2669 | GTP-binding |
| | | | | | | | protein |
| | | | | | | | overexpressed in |
| | | | | | | | skeletal muscle |
| 57 | 0.65 | 0.602 | 37639_at | X07732 | Hs.823 | 3249 | hepsin |
| | | | | | | | (transmembrane |
| | | | | | | | protease, serine 1) |
| 58 | 0.64 | 0.602 | 33730_at | AF095448 | Hs.194691 | 9052 | retinoic acid |
| | | | | | | | induced 3 |
| 59 | 0.64 | 0.602 | 37003_at | X62654 | Hs.76294 | 967 | CD63 antigen |
| | | | | | | | (melanoma 1 |
| | | | | | | | antigen) |
| 60 | 0.64 | 0.601 | 36959_at | U49278 | Hs.75875 | 7335 | ubiquitin- |
| | | | | | | | conjugating enzyme |
| | | | | | | | E2 variant 1 |
| 61 | 0.64 | 0.601 | 36488_at | AB011542 | Hs.5599 | 1955 | EGF-like-domain, |
| | | | | | | | multiple 5 |
| 62 | 0.64 | 0.601 | 37552_at | U33632 | Hs.79351 | 3775 | potassium channel, |
| | | | | | | | subfamily K, |
| | | | | | | | member 1 (TWIK- |
| | | | | | | | 1) |
| 63 | 0.64 | 0.601 | 36540_at | AB018260 | Hs.62113 | 23221 | KIAA0717 protein |
| 64 | 0.63 | 0.600 | 40031_at | M74542 | Hs.575 | 218 | aldehyde |
| | | | | | | | dehydrogenase 3 |
| 65 | 0.63 | 0.599 | 34485_r_at | M21868 | Hs.118249 | 10564 | brefeldin A- |
| | | | | | | | inhibited guanine |
| | | | | | | | nucleotide- |
| | | | | | | | exchange protein 2 |
| 66 | 0.63 | 0.599 | 206_at | M84424 | | | cathepsin E |
| 67 | 0.63 | 0.599 | 38376_at | L46590 | Hs.82208 | 37 | acyl-Coenzyme A |
| | | | | | | | dehydrogenase, |
| | | | | | | | very long chain |
| 68 | 0.63 | 0.599 | 36644_at | D29963 | Hs.75564 | 977 | CD151 antigen |
| 69 | 0.63 | 0.599 | 36963_at | U30255 | Hs.75888 | 5226 | phosphogluconate |
| | | | | | | | dehydrogenase |
| 70 | 0.62 | 0.599 | 271_s_at | J05036 | Hs.1355 | 1510 | cathepsin E |
| 71 | 0.62 | 0.599 | 36647_at | AA526812 | Hs.262823 | 55699 | hypothetical protein |
| | | | | | | | FLJ10326 |
| 72 | 0.62 | 0.599 | 32081_at | AB023166 | Hs.15767 | 11113 | citron (rho- |
| | | | | | | | interacting, |
| | | | | | | | serine/threonine |
| | | | | | | | kinase 21) |
| 73 | 0.62 | 0.598 | 691_g_at | J02783 | Hs.75655 | 5034 | procollagen-proline, |
| | | | | | | | 2-oxoglutarate 4- |
| | | | | | | | dioxygenase |
| | | | | | | | (proline 4- |
| | | | | | | | hydroxylase), beta |
| | | | | | | | polypeptide (protein |
| | | | | | | | disulfide isomerase; |
| | | | | | | | thyroid hormone |
| | | | | | | | binding protein |
| | | | | | | | p55) |
| 74 | 0.62 | 0.598 | 34835_at | D87442 | Hs.4788 | 23385 | nicastrin |
| 75 | 0.62 | 0.598 | 38642_at | Y10183 | Hs.10247 | 214 | activated leucocyte |
| | | | | | | | cell adhesion |
| | | | | | | | molecule |
| 76 | 0.62 | 0.598 | 32892_at | X85106 | Hs.301664 | 6196 | ribosomal protein |
| | | | | | | | S6 kinase, 90 kD, |
| | | | | | | | polypeptide 2 |
| 77 | 0.62 | 0.597 | 1826_at | M12174 | Hs.204354 | 388 | ras homolog gene |
| | | | | | | | family, member B |
| 78 | 0.61 | 0.597 | 38816_at | AF095791 | Hs.272023 | 10579 | transforming, acidic |
| | | | | | | | coiled-coil |
| | | | | | | | containing protein 2 |
| 79 | 0.61 | 0.597 | 39379_at | AL049397 | Hs.12314 | | clone |
| | | | | | | | DKFZp586C1019 |
| 80 | 0.61 | 0.595 | 38385_at | S65738 | Hs.82306 | 11034 | destrin (actin |
| | | | | | | | depolymerizing |
| | | | | | | | factor) |
| 81 | 0.61 | 0.595 | 39698_at | U51712 | Hs.13775 | 84525 | hypothetical protein |
| | | | | | | | SMAP31 |
| 82 | 0.61 | 0.595 | 36151_at | U60644 | Hs.74573 | 23646 | similar to vaccinia |
| | | | | | | | virus HindIII K4L |
| | | | | | | | ORF |
| 83 | 0.61 | 0.595 | 32747_at | X05409 | Hs.195432 | 217 | aldehyde |
| | | | | | | | dehydrogenase 2, |
| | | | | | | | mitochondrial |
| 84 | 0.6 | 0.594 | 39512_s_at | AA457029 | Hs.342682 | | clone RP11- |
| | | | | | | | 127K18 |

According to the invention, preferred markers are markers 1-30, preferably 1-20, and more preferably 1-10.

TABLE 4
C4 Markers
Class C4

| | | | | | UNIGENE | | |
|---|---|---|---|---|---|---|---|
| | | | | | (as of | | Desc |
| | | Perm | | GB/TIGR | summer | | (unigene/locuslink or |
| | s2n_obs | 0.1% | non_norm_list | Identifier | 2001) | LL_num | affy) |
| 1 | 1.07 | 0.786 | 1411_at | D16154 | | | cytochrome P-450c11 |
| 2 | 1.04 | 0.704 | 37021_at | X16832 | Hs.288181 | 1512 | cathepsin H |
| 3 | 1.02 | 0.701 | 534_s_at | U20391 | Hs.73769 | 2348 | folate receptor 1 |
| | | | | | | | (adult) |
| 4 | 0.95 | 0.655 | 38394_at | D42047 | Hs.82432 | 23171 | KIAA0089 protein |
| 5 | 0.94 | 0.653 | 1460_g_at | M68941 | Hs.73826 | 5775 | protein tyrosine |
| | | | | | | | phosphatase, non- |
| | | | | | | | receptor type 4 |
| | | | | | | | (megakaryocyte) |
| 6 | 0.92 | 0.650 | 33331_at | U17077 | Hs.185055 | 7851 | BENE protein |
| 7 | 0.91 | 0.648 | 38336_at | AB023230 | Hs.96427 | 23150 | KIAA1013 protein |
| 8 | 0.89 | 0.647 | 31883_at | AF025794 | Hs.153792 | 4552 | 5- |
| | | | | | | | methyltetrahydro- |
| | | | | | | | folate-homocysteine |
| | | | | | | | methyltransferase |
| | | | | | | | reductase |
| 9 | 0.88 | 0.641 | 35016_at | M13560 | | | Ia-associated |
| | | | | | | | invariant gamma- |
| | | | | | | | chain gene |
| 10 | 0.87 | 0.635 | 1629_s_at | HG3187- | | | Tyrosine |
| | | | | HT3366 | | | Phosphatase 1, Non- |
| | | | | | | | Receptor, Alt. Splice |
| | | | | | | | 3 |
| 11 | 0.87 | 0.632 | 37512_at | U89281 | Hs.11958 | 8630 | oxidative 3 alpha |
| | | | | | | | hydroxysteroid |
| | | | | | | | dehydrogenase; |
| | | | | | | | retinol |
| | | | | | | | dehydrogenase; 3- |
| | | | | | | | hydroxysteroid |
| | | | | | | | epimerase |
| 12 | 0.86 | 0.631 | 38459_g_at | L39945 | | | cytochrome b-5 |
| 13 | 0.86 | 0.631 | 36965_at | U13616 | Hs.75893 | 288 | ankyrin 3, node of |
| | | | | | | | Ranvier (ankyrin G) |
| 14 | 0.85 | 0.630 | 593_s_at | M34353 | Hs.1041 | 6098 | v-ros avian UR2 |
| | | | | | | | sarcoma virus |
| | | | | | | | oncogene homolog 1 |
| 15 | 0.85 | 0.615 | 821_s_at | U78793 | | | folate receptor 1 |
| | | | | | | | (adult) |
| 16 | 0.84 | 0.611 | 130_s_at | X82850 | Hs.197764 | 7080 | thyroid transcription |
| | | | | | | | factor 1 |
| 17 | 0.83 | 0.610 | 33278_at | AC004381 | Hs.181345 | 6296 | SA (rat hypertension- |
| | | | | | | | associated) homolog |
| 18 | 0.82 | 0.608 | 33967_at | M31525 | Hs.342656 | 3111 | major |
| | | | | | | | histocompatibility |
| | | | | | | | complex, class II, DN |
| | | | | | | | alpha |
| 19 | 0.82 | 0.605 | 35792_at | U67963 | Hs.6721 | 11343 | lysophospholipase- |
| | | | | | | | like |
| 20 | 0.81 | 0.599 | 33584_at | U35146 | Hs.158512 | 8999 | cyclin-dependent |
| | | | | | | | kinase-like 2 (CDC2- |
| | | | | | | | related kinase) |
| 21 | 0.8 | 0.598 | 38785_at | X52228 | Hs.89603 | 4582 | mucin 1, |
| | | | | | | | transmembrane |
| 22 | 0.8 | 0.597 | 34198_at | U12128 | Hs.211595 | 5783 | protein tyrosine |
| | | | | | | | phosphatase, non- |
| | | | | | | | receptor type 13 |
| | | | | | | | (APO-1/CD95 (Fas)- |
| | | | | | | | associated |
| | | | | | | | phosphatase) |
| 23 | 0.8 | 0.595 | 33249_at | M16801 | Hs.1790 | 4306 | nuclear receptor |
| | | | | | | | subfamily 3, group C, |
| | | | | | | | member 2 |
| 24 | 0.79 | 0.592 | 40310_at | AF051152 | Hs.63668 | 7097 | toll-like receptor 2 |
| 25 | 0.79 | 0.587 | 37189_at | AL023553 | Hs.75835 | 5372 | phosphomannomutase |
| | | | | | | | 1 |
| 26 | 0.79 | 0.587 | 37038_at | X83467 | Hs.76781 | 5825 | ATP-binding cassette, |
| | | | | | | | sub-family D (ALD), |
| | | | | | | | member 3 |
| 27 | 0.77 | 0.583 | 37218_at | D64110 | Hs.77311 | 10950 | BTG family, member |
| | | | | | | | 3 |
| 28 | 0.77 | 0.582 | 34823_at | X60708 | Hs.44926 | 1803 | dipeptidylpeptidase |
| | | | | | | | IV (CD26, adenosine |
| | | | | | | | deaminase |
| | | | | | | | complexing protein 2) |
| 29 | 0.77 | 0.579 | 715_s_at | D87002 | Hs.284380 | 2678 | similar to rat integral |
| | | | | | | | membrane |
| | | | | | | | glycoprotein |
| | | | | | | | POM121 |
| 30 | 0.77 | 0.578 | 38984_at | AB007896 | Hs.110 | 9581 | putative L-type |
| | | | | | | | neutral amino acid |
| | | | | | | | transporter |
| 31 | 0.77 | 0.577 | 38627_at | M95585 | Hs.250692 | 3131 | hepatic leukemia |
| | | | | | | | factor |
| 32 | 0.77 | 0.576 | 39419_at | AB011088 | Hs.129872 | 9043 | sperm associated |
| | | | | | | | antigen 9 |
| 33 | 0.76 | 0.575 | 34760_at | D14664 | Hs.2441 | 9936 | KIAA0022 gene |
| | | | | | | | product |
| 34 | 0.76 | 0.572 | 554_at | U03634 | Hs.301946 | 3928 | lymphoid blast crisis |
| | | | | | | | oncogene |
| 35 | 0.76 | 0.571 | 34996_at | U75329 | Hs.318545 | 7113 | transmembrane |
| | | | | | | | protease, serine 2 |
| 36 | 0.75 | 0.570 | 35232_f_at | AI056696 | Hs.29463 | 1070 | centrin, EF-hand |
| | | | | | | | protein, 3 (CDC31 |
| | | | | | | | yeast homolog) |
| 37 | 0.75 | 0.570 | 37886_at | AB015332 | Hs.96200 | 26993 | neighbor of A-kinase |
| | | | | | | | anchoring protein 95 |
| 38 | 0.74 | 0.570 | 36252_at | U43030 | Hs.25537 | 1489 | cardiotrophin 1 |
| 39 | 0.74 | 0.569 | 1709_g_at | U07620 | Hs.151051 | 5602 | mitogen-activated |
| | | | | | | | protein kinase 10 |
| 40 | 0.73 | 0.568 | 35221_at | X91648 | Hs.29117 | 5813 | purine-rich element |
| | | | | | | | binding protein A |
| 41 | 0.73 | 0.568 | 33933_at | X63187 | Hs.2719 | 10406 | epididymis-specific, |
| | | | | | | | whey-acidic protein |
| | | | | | | | type, four-disulfide |
| | | | | | | | core; putative ovarian |
| | | | | | | | carcinoma marker |
| 42 | 0.73 | 0.567 | 33561_at | X80031 | Hs.530 | 1285 | collagen, type IV, |
| | | | | | | | alpha 3 (Goodpasture |
| | | | | | | | antigen) |
| 43 | 0.73 | 0.566 | 41809_at | AI656421 | Hs.322404 | 79161 | hypothetical protein |
| | | | | | | | MGC4175 |
| 44 | 0.73 | 0.566 | 36511_at | AB020658 | Hs.5867 | 22908 | KIAA0851 protein |
| 45 | 0.73 | 0.565 | 41109_at | M31452 | Hs.1012 | 722 | complement |
| | | | | | | | component 4-binding |
| | | | | | | | protein, alpha |
| 46 | 0.72 | 0.562 | 32893_s_at | M30474 | Hs.289098 | 2679 | gamma- |
| | | | | | | | glutamyltransferase 2 |
| 47 | 0.72 | 0.561 | 39345_at | AI525834 | Hs.119529 | 10577 | Niemann-Pick |
| | | | | | | | disease, type C2 gene |
| 48 | 0.72 | 0.559 | 39115_at | AL050275 | Hs.9383 | 25982 | DKFZP566D213 |
| | | | | | | | protein |
| 49 | 0.72 | 0.558 | 40508_at | AF025887 | Hs.169907 | 2941 | glutathione S- |
| | | | | | | | transferase A4 |
| 50 | 0.71 | 0.557 | 1137_at | L20852 | Hs.10018 | 6575 | solute carrier family |
| | | | | | | | 20 (phosphate |
| | | | | | | | transporter), member |
| | | | | | | | 2 |
| 51 | 0.71 | 0.557 | 40101_g_at | U72206 | Hs.337774 | 9181 | rho/rac guanine |
| | | | | | | | nucleotide exchange |
| | | | | | | | factor (GEF) 2 |
| 52 | 0.7 | 0.556 | 711_at | HG2339- | | | Nuclear Factor 1, |
| | | | | HT2435 | | | Variant Hepatic |
| 53 | 0.7 | 0.555 | 40834_at | AB002298 | Hs.173035 | 23037 | KIAA0300 protein |
| 54 | 0.7 | 0.554 | 41302_at | R59606 | Hs.4113 | 10768 | S- |
| | | | | | | | adenosylhomocysteine |
| | | | | | | | hydrolase-like 1 |
| 55 | 0.69 | 0.552 | 1922_g_at | HG2510- | | | Ras-Specific Guanine |
| | | | | HT2606 | | | Nucleotide-Releasing |
| | | | | | | | Factor |
| 56 | 0.69 | 0.552 | 37579_at | L47738 | Hs.258503 | 26999 | p53 inducible protein |
| 57 | 0.69 | 0.551 | 32902_at | U28281 | Hs.2199 | 6344 | secretin receptor |
| 58 | 0.69 | 0.548 | 704_at | HG4167- | | | Nuclear Factor 1, A |
| | | | | HT4437 | | | Type |
| 59 | 0.69 | 0.547 | 37676_at | AF056490 | Hs.78746 | 5151 | phosphodiesterase 8A |
| 60 | 0.69 | 0.547 | 33621_at | X71348 | | | transcription factor 2, |
| | | | | | | | hepatic; LF-B3; |
| | | | | | | | variant hepatic |
| | | | | | | | nuclear factor |
| 61 | 0.69 | 0.547 | 38252_s_at | U84007 | Hs.904 | 178 | amylo-1,6- |
| | | | | | | | glucosidase, 4-alpha- |
| | | | | | | | glucanotransferase |
| | | | | | | | (glycogen |
| | | | | | | | debranching enzyme, |
| | | | | | | | glycogen storage |
| | | | | | | | disease type III) |
| 62 | 0.68 | 0.544 | 34213_at | AB020676 | Hs.21543 | 23286 | KIAA0869 protein |
| 63 | 0.68 | 0.544 | 37405_at | U29091 | Hs.334841 | 8991 | selenium binding |
| | | | | | | | protein 1 |
| 64 | 0.68 | 0.543 | 34767_at | AI670788 | Hs.24719 | 64112 | modulator of |
| | | | | | | | apoptosis 1 |
| 65 | 0.68 | 0.542 | 35955_at | S80864 | Hs.262219 | 25835 | cytochrome c-like |
| | | | | | | | antigen |
| 66 | 0.68 | 0.541 | 38790_at | L25879 | Hs.89649 | 2052 | epoxide hydrolase 1, |
| | | | | | | | microsomal |
| | | | | | | | (xenobiotic) |
| 67 | 0.68 | 0.540 | 36508_at | AF030186 | Hs.58367 | 2239 | glypican 4 |
| 68 | 0.68 | 0.540 | 33942_s_at | AF004563 | Hs.239356 | 6812 | syntaxin binding |
| | | | | | | | protein 1 |
| 69 | 0.67 | 0.540 | 37629_at | M55268 | Hs.82201 | 1459 | casein kinase 2, alpha |
| | | | | | | | prime polypeptide |
| 70 | 0.67 | 0.539 | 32822_at | J02966 | Hs.2043 | 291 | solute carrier family |
| | | | | | | | 25 (mitochondrial |
| | | | | | | | carrier; adenine |
| | | | | | | | nucleotide |
| | | | | | | | translocator), member |
| | | | | | | | 4 |
| 71 | 0.67 | 0.538 | 35472_at | Y10745 | Hs.17287 | 3772 | potassium inwardly- |
| | | | | | | | rectifying channel, |
| | | | | | | | subfamily J, member |
| | | | | | | | 15 |
| 72 | 0.67 | 0.537 | 34163_g_at | D84111 | Hs.80248 | 11030 | RNA-binding protein |
| | | | | | | | gene with multiple |
| | | | | | | | splicing |
| 73 | 0.67 | 0.536 | 31925_s_at | L26584 | Hs.169350 | 5923 | Ras protein-specific |
| | | | | | | | guanine nucleotide- |
| | | | | | | | releasing factor 1 |
| 74 | 0.67 | 0.536 | 32854_at | AB014596 | Hs.21229 | 23291 | f-box and WD-40 |
| | | | | | | | domain protein 1B |
| 75 | 0.67 | 0.535 | 35645_at | AL050148 | Hs.31834 | | clone |
| | | | | | | | DKFZp586G1520 |
| 76 | 0.66 | 0.535 | 1986_at | X74594 | Hs.79362 | 5934 | retinoblastoma-like 2 |
| | | | | | | | (p130) |
| 77 | 0.66 | 0.533 | 1938_at | K03218 | | | v-src avian sarcoma |
| | | | | | | | (Schmidt-Ruppin A- |
| | | | | | | | 2) viral oncogene |
| | | | | | | | homolog |
| 78 | 0.66 | 0.532 | 1616_at | D14838 | Hs.111 | 2254 | fibroblast growth |
| | | | | | | | factor 9 (glia- |
| | | | | | | | activating factor) |
| 79 | 0.66 | 0.532 | 41440_at | D82061 | Hs.288354 | 7923 | FabG (beta-ketoacyl- |
| | | | | | | | [acyl-carrier-protein] |
| | | | | | | | reductase, E coli) like |
| 80 | 0.66 | 0.530 | 41129_at | D26067 | Hs.174905 | 23027 | KIAA0033 protein |
| 81 | 0.66 | 0.530 | 40209_at | U72671 | Hs.151250 | 7087 | intercellular adhesion |
| | | | | | | | molecule 5, |
| | | | | | | | telencephalin |
| 82 | 0.65 | 0.529 | 32676_at | M93405 | Hs.293970 | 4329 | methylmalonate- |
| | | | | | | | semialdehyde |
| | | | | | | | dehydrogenase |
| 83 | 0.65 | 0.528 | 36557_at | M92303 | Hs.635 | 782 | calcium channel, |
| | | | | | | | voltage-dependent, |
| | | | | | | | beta 1 subunit |
| 84 | 0.65 | 0.528 | 35228_at | Y08682 | Hs.29331 | 1375 | carnitine |
| | | | | | | | palmitoyltransferase |
| | | | | | | | I, muscle |
| 85 | 0.65 | 0.527 | 1667_s_at | J02871 | Hs.687 | 1580 | cytochrome P450, |
| | | | | | | | subfamily IVB, |
| | | | | | | | polypeptide 1 |
| 86 | 0.65 | 0.526 | 40701_at | U75362 | Hs.85482 | 8975 | ubiquitin specific |
| | | | | | | | protease 13 |
| | | | | | | | (isopeptidase T-3) |
| 87 | 0.65 | 0.525 | 40343_at | AJ005814 | Hs.70954 | 3204 | homeo box A7 |
| 88 | 0.65 | 0.524 | 39301_at | X85030 | Hs.40300 | 825 | calpain 3, (p94) |
| 89 | 0.65 | 0.524 | 35435_s_at | AF001903 | Hs.8110 | 3033 | L-3-hydroxyacyl- |
| | | | | | | | Coenzyme A |
| | | | | | | | dehydrogenase, short |
| | | | | | | | chain |
| 90 | 0.64 | 0.523 | 34235_at | AB018301 | Hs.22039 | 23282 | KIAA0758 protein |
| 91 | 0.64 | 0.523 | 37344_at | X62744 | Hs.77522 | 3108 | major |
| | | | | | | | histocompatibility |
| | | | | | | | complex, class II, DM |
| | | | | | | | alpha |
| 92 | 0.64 | 0.522 | 41120_at | D14686 | | | aminomethyltransferase |
| | | | | | | | (glycine cleavage |
| | | | | | | | system protein T) |
| 93 | 0.64 | 0.522 | 40673_at | U12778 | Hs.81934 | 36 | acyl-Coenzyme A |
| | | | | | | | dehydrogenase, |
| | | | | | | | short/branched chain |
| 94 | 0.63 | 0.521 | 34353_at | AB014548 | Hs.31921 | 23244 | KIAA0648 protein |
| 95 | 0.63 | 0.520 | 35285_at | AF007216 | Hs.5462 | 8671 | solute carrier family |
| | | | | | | | 4, sodium bicarbonate |
| | | | | | | | cotransporter, |
| | | | | | | | member 4 |
| 96 | 0.63 | 0.520 | 40822_at | L41067 | Hs.172674 | 4775 | nuclear factor of |
| | | | | | | | activated T-cells, |
| | | | | | | | cytoplasmic, |
| | | | | | | | calcineurin-dependent |
| | | | | | | | 3 |
| 97 | 0.63 | 0.519 | 41331_at | R93981 | Hs.24279 | 9860 | KIAA0806 gene |
| | | | | | | | product |
| 98 | 0.63 | 0.519 | 40278_at | AB029003 | Hs.155546 | 23062 | KIAA1080 protein; |
| | | | | | | | Golgi-associated, |
| | | | | | | | gamma-adaptin ear |
| | | | | | | | containing, ARF- |
| | | | | | | | binding protein 2 |
| 99 | 0.63 | 0.519 | 36828_at | AB002324 | Hs.301094 | 23361 | KIAA0326 protein |
| 100 | 0.63 | 0.519 | 40128_at | D79993 | Hs.132853 | 9685 | KIAA0171 gene |
| | | | | | | | product |
| 101 | 0.63 | 0.519 | 35382_at | AF043244 | Hs.278439 | 8996 | nucleolar protein 3 |
| | | | | | | | (apoptosis repressor |
| | | | | | | | with CARD domain) |
| 102 | 0.63 | 0.518 | 40217_s_at | U65887 | Hs.152981 | 1040 | CDP-diacylglycerol |
| | | | | | | | synthase |
| | | | | | | | (phosphatidate |
| | | | | | | | cytidylyltransferase) |
| | | | | | | | 1 |
| 103 | 0.63 | 0.518 | 38095_i_at | M83664 | Hs.814 | 3115 | major |
| | | | | | | | histocompatibility |
| | | | | | | | complex, class II, DP |
| | | | | | | | beta 1 |
| 104 | 0.62 | 0.518 | 34555_at | X63755 | Hs.2743 | 3846 | keratin, cuticle, |
| | | | | | | | ultrahigh sulphur 1 |
| 105 | 0.62 | 0.517 | 33263_at | X67098 | | | rTS beta protein |
| 106 | 0.62 | 0.517 | 33267_at | AF035315 | Hs.180737 | | clone 23664 and |
| | | | | | | | 23905 |
| 107 | 0.62 | 0.517 | 1594_at | J05448 | Hs.79402 | 5432 | polymerase (RNA) II |
| | | | | | | | (DNA directed) |
| | | | | | | | polypeptide C (33 kD) |
| 108 | 0.62 | 0.516 | 40013_at | Y12696 | Hs.54570 | 1193 | chloride intracellular |
| | | | | | | | channel 2 |
| 109 | 0.62 | 0.516 | 32122_at | L31573 | Hs.16340 | 6821 | sulfite oxidase |
| 110 | 0.62 | 0.515 | 34800_at | AL039458 | Hs.4193 | 26018 | ortholog of mouse |
| | | | | | | | integral membrane |
| | | | | | | | glycoprotein LIG-1 |
| 111 | 0.62 | 0.515 | 41723_s_at | M32578 | Hs.180255 | 3123 | major |
| | | | | | | | histocompatibility |
| | | | | | | | complex, class II, DR |
| | | | | | | | beta 1 |
| 112 | 0.62 | 0.515 | 38683_s_at | AB029008 | Hs.301226 | 57450 | KIAA1085 protein |
| 113 | 0.62 | 0.514 | 32235_at | AB011116 | Hs.284251 | 23295 | KIAA0544 protein |
| 114 | 0.62 | 0.514 | 41689_at | R16035 | Hs.12701 | 51090 | plasmolipin |
| 115 | 0.62 | 0.514 | 38318_at | AL050128 | Hs.95260 | 51439 | Autosomal Highly |
| | | | | | | | Conserved Protein |
| 116 | 0.61 | 0.513 | 1619_g_at | D21241 | | | cytochrome P-450 |
| | | | | | | | aromatase |
| 117 | 0.61 | 0.513 | 39266_at | AF070632 | Hs.23729 | | clone 24405 |
| 118 | 0.61 | 0.513 | 40711_at | AL049340 | Hs.86405 | | clone |
| | | | | | | | DKFZp564P056 |
| 119 | 0.61 | 0.512 | 39247_at | U66689 | Hs.274260 | 368 | ATP-binding cassette, |
| | | | | | | | sub-family C |
| | | | | | | | (CFTR/MRP), |
| | | | | | | | member 6 |
| 120 | 0.61 | 0.512 | 39820_at | AF001549 | Hs.110103 | 54700 | RNA polymerase I |
| | | | | | | | transcription factor |
| | | | | | | | RRN3 |
| 121 | 0.61 | 0.511 | 39974_at | AF039917 | Hs.47042 | 956 | ectonucleoside |
| | | | | | | | triphosphate |
| | | | | | | | diphosphohydrolase 3 |
| 122 | 0.61 | 0.511 | 37704_at | Z14093 | Hs.78950 | 593 | branched chain keto |
| | | | | | | | acid dehydrogenase |
| | | | | | | | E1, alpha polypeptide |
| | | | | | | | (maple syrup urine |
| | | | | | | | disease) |
| 123 | 0.61 | 0.510 | 34521_at | AB001872 | Hs.21291 | 9175 | mitogen-activated |
| | | | | | | | protein kinase kinase |
| | | | | | | | kinase 13 |
| 124 | 0.6 | 0.509 | 38072_at | AL031432 | Hs.8084 | 57035 | hypothetical protein |
| | | | | | | | dJ465N24.2.1 |
| 125 | 0.6 | 0.509 | 40149_at | AL049924 | Hs.15744 | 25970 | SH2-B homolog |
| 126 | 0.6 | 0.509 | 39138_g_at | X80878 | Hs.95262 | 4798 | nuclear factor related |
| | | | | | | | to kappa B binding |
| | | | | | | | protein |
| 127 | 0.6 | 0.508 | 38064_at | X79882 | Hs.80680 | 9961 | major vault protein |
| 128 | 0.6 | 0.508 | 34473_at | AF051151 | Hs.114408 | 7100 | toll-like receptor 5 |
| 129 | 0.6 | 0.508 | 36755_s_at | M75914 | Hs.68876 | 3568 | Interleukin 5 receptor, |
| | | | | | | | alpha |
| 130 | 0.6 | 0.507 | 41686_s_at | AL042668 | Hs.337629 | | cDNA, 5 end |
| 131 | 0.6 | 0.507 | 41424_at | L48516 | Hs.296259 | 5446 | paraoxonase 3 |
| 132 | 0.6 | 0.507 | 903_at | L42373 | Hs.155079 | 5525 | protein phosphatase |
| | | | | | | | 2, regulatory subunit |
| | | | | | | | B (B56), alpha |
| | | | | | | | isoform |
| 133 | 0.6 | 0.506 | 35408_i_at | X16281 | Hs.278480 | 7595 | zinc finger protein 44 |
| | | | | | | | (KOX 7) |
| 134 | 0.59 | 0.506 | 1270_at | M64788 | Hs.75151 | 5909 | RAP1, GTPase |
| | | | | | | | activating protein 1 |
| 135 | 0.59 | 0.506 | 1087_at | M60459 | Hs.89548 | 2057 | erythropoietin |
| | | | | | | | receptor |
| 136 | 0.59 | 0.505 | 33290_at | M74161 | Hs.182577 | 3633 | inositol |
| | | | | | | | polyphosphate-5- |
| | | | | | | | phosphatase, 75 kD |
| 137 | 0.59 | 0.505 | 39408_at | Z80345 | Hs.127610 | 35 | acyl-Coenzyme A |
| | | | | | | | dehydrogenase, C-2 |
| | | | | | | | to C-3 short chain |
| 138 | 0.59 | 0.505 | 40766_at | U24578 | Hs.278625 | 721 | complement |
| | | | | | | | component 4B |
| 139 | 0.59 | 0.505 | 39612_at | AL050061 | Hs.27371 | | clone DKFZp566J123 |
| 140 | 0.59 | 0.504 | 38850_at | M11119 | Hs.272951 | | endogenous retrovirus |
| | | | | | | | envelope region |
| | | | | | | | mRNA (PL1) |
| 141 | 0.59 | 0.504 | 34529 at | W26760 | Hs.336635 | | cDNA |
| 142 | 0.59 | 0.504 | 40394_at | L17128 | Hs.77719 | 2677 | gamma-glutamyl |
| | | | | | | | carboxylase |
| 143 | 0.59 | 0.503 | 37811_at | AF042792 | Hs.127436 | 9254 | calcium channel, |
| | | | | | | | voltage-dependent, |
| | | | | | | | alpha 2/delta subunit |
| | | | | | | | 2 |
| 144 | 0.58 | 0.503 | 37150_at | AB026190 | Hs.106290 | 27252 | Kelch motif |
| | | | | | | | containing protein |
| 145 | 0.58 | 0.503 | 41346_at | AJ007583 | Hs.25220 | 9215 | like- |
| | | | | | | | glycosyltransferase |
| 146 | 0.58 | 0.502 | 37609_at | U01833 | Hs.81469 | 4682 | nucleotide binding |
| | | | | | | | protein 1 (E. coli |
| | | | | | | | MinD like) |
| 147 | 0.58 | 0.502 | 35988_i_at | AI417075 | Hs.42343 | 84148 | hypothetical protein |
| | | | | | | | FLJ14040 |
| 148 | 0.58 | 0.501 | 32427_at | U66583 | Hs.72911 | 1421 | crystallin, gamma D |
| 149 | 0.58 | 0.501 | 37151_at | AF052120 | Hs.106334 | | clone 23836 |
| 150 | 0.58 | 0.501 | 37172_at | M75106 | Hs.75572 | 1361 | carboxypeptidase B2 |
| | | | | | | | (plasma) |
| 151 | 0.58 | 0.500 | 35815_at | AL049470 | Hs.306184 | 25767 | Huntingtin interacting |
| | | | | | | | protein B |
| 152 | 0.58 | 0.499 | 37722_s_at | U26266 | Hs.79064 | 1725 | deoxyhypusine |
| | | | | | | | synthase |
| 153 | 0.58 | 0.499 | 40600_at | AW024467 | Hs.172847 | 3338 | DnaJ (Hsp40) |
| | | | | | | | homolog, subfamily |
| | | | | | | | C, member 4 |
| 154 | 0.57 | 0.499 | 38086_at | AB007935 | Hs.81234 | 3321 | immunoglobulin |
| | | | | | | | superfamily, member |
| | | | | | | | 3 |
| 155 | 0.57 | 0.499 | 38285_at | AF039397 | | | crystallin, mu |
| 156 | 0.57 | 0.499 | 41381_at | AB002306 | Hs.10351 | 23337 | KIAA0308 protein |
| 157 | 0.57 | 0.498 | 34716_at | AF067730 | Hs.3530 | 63902 | TLS-associated |
| | | | | | | | serine-arginine |
| | | | | | | | protein 2 |
| 158 | 0.57 | 0.498 | 38492_at | D55639 | Hs.169139 | 8942 | kynureninase (L- |
| | | | | | | | kynurenine |
| | | | | | | | hydrolase) |
| 159 | 0.57 | 0.497 | 39438_at | AF039081 | Hs.13313 | 1389 | cAMP responsive |
| | | | | | | | element binding |
| | | | | | | | protein-like 2 |
| 160 | 0.57 | 0.497 | 36997_at | J04809 | Hs.76240 | 203 | adenylate kinase 1 |
| 161 | 0.57 | 0.497 | 32076_at | D83407 | Hs.156007 | 10231 | Down syndrome |
| | | | | | | | critical region gene 1- |
| | | | | | | | like 1 |
| 162 | 0.57 | 0.497 | 32185_at | U00946 | Hs.184592 | 65125 | protein kinase, lysine |
| | | | | | | | deficient 1 |
| 163 | 0.57 | 0.496 | 36538_at | AB018314 | Hs.6162 | 23368 | KIAA0771 protein |
| 164 | 0.56 | 0.496 | 41339_at | AF043117 | Hs.24594 | 10277 | ubiquitination factor |
| | | | | | | | E4B (homologous to |
| | | | | | | | yeast UFD2) |
| 165 | 0.56 | 0.495 | 32144_at | AL050135 | Hs.166891 | 5993 | regulatory factor X, 5 |
| | | | | | | | (influences HLA |
| | | | | | | | class II expression) |
| 166 | 0.56 | 0.495 | 37402_at | D26129 | Hs.78224 | 6035 | ribonuclease, RNase |
| | | | | | | | A family, 1 |
| | | | | | | | (pancreatic) |
| 167 | 0.56 | 0.494 | 700_s_at | HG371- | | | Mucin 1, Epithelial, |
| | | | | HT26388 | | | Alt. Splice 9 |
| 168 | 0.56 | 0.494 | 33521_at | M63962 | Hs.36992 | 495 | ATPase, H+/K+ |
| | | | | | | | exchanging, alpha |
| | | | | | | | polypeptide |
| 169 | 0.56 | 0.494 | 34934_at | L29376 | Hs.132807 | | (clone 3.8-1) MHC |
| | | | | | | | class I |
| 170 | 0.56 | 0.494 | 41018_at | AL050015 | Hs.92700 | 25864 | DKFZP564O243 |
| | | | | | | | protein |
| 171 | 0.56 | 0.493 | 37539_at | AB023176 | Hs.79219 | 23179 | RalGDS-like gene; |
| | | | | | | | KIAA0959 protein |
| 172 | 0.56 | 0.493 | 36626_at | X87176 | Hs.75441 | 3295 | hydroxysteroid (17- |
| | | | | | | | beta) dehydrogenase |
| | | | | | | | 4 |
| 173 | 0.56 | 0.493 | 36012_at | Y09631 | Hs.43913 | 10464 | PIBF1 gene product |
| 174 | 0.56 | 0.493 | 41491_s_at | AB028944 | Hs.29189 | 23250 | ATPase, Class VI, |
| | | | | | | | type 11A |
| 175 | 0.56 | 0.493 | 32746_at | AF015451 | Hs.195175 | 8837 | CASP8 and FADD- |
| | | | | | | | like apoptosis |
| | | | | | | | regulator |
| 176 | 0.56 | 0.492 | 40833_r_at | AL050126 | Hs.234265 | 26092 | DKFZP586G011 |
| | | | | | | | protein |
| 177 | 0.56 | 0.492 | 34256_at | AB018356 | Hs.225939 | 8869 | sialyltransferase 9 |
| | | | | | | | (CMP- |
| | | | | | | | NeuAc: lactosyl- |
| | | | | | | | ceramide alpha-2,3- |
| | | | | | | | sialyltransferase; |
| | | | | | | | GM3 synthase) |
| 178 | 0.56 | 0.491 | AFFX- | L38424 | | | B subtilis dapB, jojF, |
| | | | DapX-M_at | | | | jojG genes |
| | | | | | | | corresponding to |
| | | | | | | | nucleotides 1358- |
| | | | | | | | 3197 of L38424 |
| | | | | | | | (−5, −M, |
| | | | | | | | −3 represent |
| | | | | | | | transcript regions 5 |
| | | | | | | | prime, Middle, and 3 |
| | | | | | | | prime respectively) |
| 179 | 0.55 | 0.491 | 40547_at | AI688516 | Hs.163867 | 4695 | NADH |
| | | | | | | | dehydrogenase |
| | | | | | | | (ubiquinone) 1 alpha |
| | | | | | | | subcomplex, 2 (8 kD, |
| | | | | | | | B8) |
| 180 | 0.55 | 0.491 | 41488_at | AC002394 | Hs.144852 | | hypothetical protein |
| | | | | | | | A-211C6.1 |
| 181 | 0.55 | 0.491 | 41501_at | AF004849 | Hs.30148 | 10114 | homeodomain- |
| | | | | | | | interacting protein |
| | | | | | | | kinase 3 |
| 182 | 0.55 | 0.490 | 35287_at | AF046888 | Hs.54673 | 8741 | tumor necrosis factor |
| | | | | | | | (ligand) superfamily, |
| | | | | | | | member 13 |
| 183 | 0.55 | 0.490 | 33284_at | M19507 | Hs.1817 | 4353 | myeloperoxidase |
| 184 | 0.55 | 0.490 | 40152_r_at | Z48054 | Hs.158084 | 5830 | peroxisome receptor |
| | | | | | | | 1 |
| 185 | 0.55 | 0.490 | 34001_at | AF033199 | Hs.8198 | 7754 | zinc finger protein |
| | | | | | | | 204 |
| 186 | 0.55 | 0.489 | 1527_s_at | U50527 | Hs.22174 | | BRCA2 region |
| 187 | 0.55 | 0.489 | 34141_at | AL109681 | Hs.226017 | | clone EUROIMAGE |
| | | | | | | | 112333 |
| 188 | 0.55 | 0.489 | 34116_at | AF038852 | Hs.21903 | 785 | calcium channel, |
| | | | | | | | voltage-dependent, |
| | | | | | | | beta 4 subunit |
| 189 | 0.55 | 0.488 | 36806_at | X83877 | Hs.289104 | 11256 | Alu-binding protein |
| | | | | | | | with zinc finger |
| | | | | | | | domain |
| 190 | 0.55 | 0.488 | 39557_at | AI625844 | Hs.295963 | | cDNA, 3 end |
| 191 | 0.55 | 0.487 | 40595_at | AI345337 | Hs.301266 | 6949 | Treacher Collins- |
| | | | | | | | Franceschetti |
| | | | | | | | syndrome 1 |
| 192 | 0.55 | 0.487 | 39993_at | D11466 | Hs.51 | 5277 | phosphatidylinositol |
| | | | | | | | glycan, class A |
| | | | | | | | (paroxysmal |
| | | | | | | | nocturnal |
| | | | | | | | hemoglobinuria) |
| 193 | 0.55 | 0.487 | 39947_at | AJ006352 | Hs.42331 | 1945 | ephrin-A4 |
| 194 | 0.55 | 0.487 | 785_at | U96114 | Hs.315493 | 11060 | Nedd-4-like |
| | | | | | | | ubiquitin-protein |
| | | | | | | | ligase |
| 195 | 0.55 | 0.487 | 33569_at | D50532 | Hs.54403 | 10462 | macrophage lectin 2 |
| | | | | | | | (calcium dependent) |
| 196 | 0.54 | 0.486 | 39171_at | W21787 | Hs.99816 | 56998 | beta-catenin- |
| | | | | | | | interacting protein |
| | | | | | | | ICAT |
| 197 | 0.54 | 0.486 | 39678_at | D10511 | | | acetyl-Coenzyme A |
| | | | | | | | acetyltransferase 1 |
| | | | | | | | (acetoacetyl |
| | | | | | | | Coenzyme A |
| | | | | | | | thiolase) |
| 198 | 0.54 | 0.486 | 881_at | M35198 | Hs.123125 | 3694 | integrin, beta 6 |
| 199 | 0.54 | 0.485 | 40064_at | AB011121 | Hs.154248 | 66008 | amyotrophic lateral |
| | | | | | | | sclerosis 2 (juvenile) |
| | | | | | | | chromosome region, |
| | | | | | | | candidate 3 |
| 200 | 0.54 | 0.485 | 33800_at | AF036927 | Hs.20196 | 115 | adenylate cyclase 9 |

According to the invention, preferred markers are markers 1-30, preferably 1-20, and more preferably 1-10. Highly preferred markers are cathepsin H, folate receptor 1 (adult), BENE protein, and cytochrome b-5.

TABLE 5
Normal Lung Markers
Class Norm

| | | | | | UNIGENE | | |
|---|---|---|---|---|---|---|---|
| | | | | | (as of | | Desc |
| | | Perm | | GB/TIGR | summer | | (unigene/locuslink or |
| | s2n_obs | 0.1% | non_norm_list | Identifier | 2001) | LL_num | affy) |
| 1 | 1.97 | 0.677 | 32542_at | AF063002 | Hs.239069 | 2273 | four and a half LIM |
| | | | | | | | domains 1 |
| 2 | 1.85 | 0.631 | 1815_g_at | D50683 | Hs.82028 | 7048 | transforming growth |
| | | | | | | | factor, beta receptor II |
| | | | | | | | (70-80 kD) |
| 3 | 1.82 | 0.626 | 36119_at | AF070648 | Hs.74034 | | clone 24651 |
| 4 | 1.75 | 0.603 | 35868_at | M91211 | Hs.184 | 177 | advanced |
| | | | | | | | glycosylation end |
| | | | | | | | product-specific |
| | | | | | | | receptor |
| 5 | 1.71 | 0.600 | 39031_at | AA152406 | Hs.114346 | 1346 | cytochrome c oxidase |
| | | | | | | | subunit VIIa |
| | | | | | | | polypeptide 1 (muscle) |
| 6 | 1.7 | 0.594 | 37398_at | AA100961 | Hs.78146 | 5175 | platelet/endothelial |
| | | | | | | | cell adhesion molecule |
| | | | | | | | (CD31 antigen) |
| 7 | 1.7 | 0.592 | 40331_at | AF035819 | Hs.67726 | 8685 | macrophage receptor |
| | | | | | | | with collagenous |
| | | | | | | | structure |
| 8 | 1.7 | 0.589 | 40607_at | U97105 | Hs.173381 | 1808 | dihydropyrimidinase- |
| | | | | | | | like 2 |
| 9 | 1.7 | 0.588 | 40841_at | AF049910 | Hs.173159 | 6867 | transforming, acidic |
| | | | | | | | coiled-coil containing |
| | | | | | | | protein 1 |
| 10 | 1.69 | 0.587 | 38454_g_at | X15606 | Hs.83733 | 3384 | intercellular adhesion |
| | | | | | | | molecule 2 |
| 11 | 1.65 | 0.582 | 36569_at | X64559 | Hs.65424 | 7123 | tetranectin |
| | | | | | | | (plasminogen-binding |
| | | | | | | | protein) |
| 12 | 1.63 | 0.578 | 39066_at | L38486 | Hs.296049 | 4239 | microfibrillar- |
| | | | | | | | associated protein 4 |
| 13 | 1.6 | 0.576 | 40282_s_at | M84526 | Hs.155597 | 1675 | D component of |
| | | | | | | | complement (adipsin) |
| 14 | 1.6 | 0.575 | 34320_at | AL050224 | Hs.29759 | 22939 | polymerase I and |
| | | | | | | | transcript release |
| | | | | | | | factor |
| 15 | 1.6 | 0.574 | 37027_at | M80899 | Hs.301417 | 195 | AHNAK |
| | | | | | | | nucleoprotein |
| | | | | | | | (desmoyokin) |
| 16 | 1.58 | 0.574 | 33328_at | W28612 | Hs.296326 | | cDNA |
| 17 | 1.58 | 0.573 | 35985_at | AB023137 | Hs.42322 | 11217 | A kinase (PRKA) |
| | | | | | | | anchor protein 2 |
| 18 | 1.57 | 0.572 | 770_at | D00632 | Hs.336920 | 2878 | glutathione peroxidase |
| | | | | | | | 3 (plasma) |
| 19 | 1.55 | 0.570 | 38177_at | AJ001015 | Hs.155106 | 10266 | receptor (calcitonin) |
| | | | | | | | activity modifying |
| | | | | | | | protein 2 |
| 20 | 1.54 | 0.568 | 39760_at | AL031781 | Hs.15020 | 9444 | homolog of mouse |
| | | | | | | | quaking QKI (KH |
| | | | | | | | domain RNA binding |
| | | | | | | | protein) |
| 21 | 1.54 | 0.567 | 268_at | L34657 | | | platelet/endothelial |
| | | | | | | | cell adhesion molecule |
| | | | | | | | (CD31 antigen) |
| 22 | 1.53 | 0.567 | 33756_at | U39447 | Hs.198241 | 8639 | amine oxidase, copper |
| | | | | | | | containing 3 (vascular |
| | | | | | | | adhesion protein 1) |
| 23 | 1.51 | 0.567 | 32562_at | X72012 | Hs.76753 | 2022 | endoglin (Osler- |
| | | | | | | | Rendu-Weber |
| | | | | | | | syndrome 1) |
| 24 | 1.51 | 0.566 | 40419_at | X85116 | Hs.160483 | 2040 | erythrocyte membrane |
| | | | | | | | protein band 7.2 |
| | | | | | | | (stomatin) |
| 25 | 1.48 | 0.565 | 40994_at | L15388 | Hs.211569 | 2869 | G protein-coupled |
| | | | | | | | receptor kinase 5 |
| 26 | 1.48 | 0.564 | 38430_at | AA128249 | Hs.83213 | 2167 | fatty acid binding |
| | | | | | | | protein 4, adipocyte |
| 27 | 1.47 | 0.564 | 36155_at | D87465 | Hs.74583 | 9806 | KIAA0275 gene |
| | | | | | | | product |
| 28 | 1.47 | 0.564 | 39631_at | U52100 | Hs.29191 | 2013 | epithelial membrane |
| | | | | | | | protein 2 |
| 29 | 1.45 | 0.563 | 36627_at | X86693 | Hs.75445 | 8404 | SPARC-like 1 (mast9, |
| | | | | | | | hevin) |
| 30 | 1.45 | 0.562 | 35730_at | X03350 | Hs.4 | 125 | alcohol dehydrogenase |
| | | | | | | | 2 (class I), beta |
| | | | | | | | polypeptide |
| 31 | 1.42 | 0.561 | 34708_at | D88587 | Hs.333383 | 8547 | ficolin |
| | | | | | | | (collagen/fibrinogen |
| | | | | | | | domain-containing) 3 |
| | | | | | | | (Hakata antigen) |
| 32 | 1.42 | 0.560 | 39775_at | X54486 | Hs.151242 | 710 | serine (or cysteine) |
| | | | | | | | proteinase inhibitor, |
| | | | | | | | clade G (C1 inhibitor), |
| | | | | | | | member 1 |
| 33 | 1.41 | 0.560 | 38239_at | AI312905 | Hs.16762 | | cDNA, 3 end |
| 34 | 1.41 | 0.559 | 35261_at | W07033 | Hs.5210 | 9535 | glia maturation factor, |
| | | | | | | | gamma |
| 35 | 1.4 | 0.559 | 39350_at | U50410 | Hs.119651 | 2719 | glypican 3 |
| 36 | 1.39 | 0.559 | 40560_at | U28049 | Hs.168357 | 6909 | T-box 2 |
| 37 | 1.39 | 0.559 | 607_s_at | M10321 | Hs.110802 | 7450 | von Willebrand factor |
| 38 | 1.36 | 0.557 | 1596_g_at | L06139 | Hs.89640 | 7010 | TEK tyrosine kinase, |
| | | | | | | | endothelial (venous |
| | | | | | | | malformations, |
| | | | | | | | multiple cutaneous and |
| | | | | | | | mucosal) |
| 39 | 1.36 | 0.557 | 38653_at | D11428 | Hs.103724 | 5376 | peripheral myelin |
| | | | | | | | protein 22 |
| 40 | 1.35 | 0.557 | 36577_at | Z24725 | Hs.75260 | 10979 | mitogen inducible 2 |
| 41 | 1.33 | 0.555 | 37976_at | AL034397 | Hs.8904 | 11326 | Ig superfamily protein |
| 42 | 1.33 | 0.554 | 34210_at | N90866 | Hs.276770 | 1043 | CDW52 antigen |
| | | | | | | | (CAMPATH-1 |
| | | | | | | | antigen) |
| 43 | 1.33 | 0.554 | 38508_s_at | U89337 | Hs.169886 | 7148 | DIR1 protein |
| 44 | 1.32 | 0.553 | 32780_at | AB018271 | Hs.198689 | 26029 | KIAA0728 protein |
| 45 | 1.31 | 0.553 | 39634_at | AB017168 | Hs.29802 | 9353 | slit (Drosophila) |
| | | | | | | | homolog 2 |
| 46 | 1.31 | 0.552 | 38995_at | AF000959 | Hs.110903 | 7122 | claudin 5 |
| | | | | | | | (transmembrane |
| | | | | | | | protein deleted in |
| | | | | | | | velocardiofacial |
| | | | | | | | syndrome) |
| 47 | 1.3 | 0.552 | 37099_at | AI806222 | Hs.100194 | 241 | arachidonate 5- |
| | | | | | | | lipoxygenase- |
| | | | | | | | activating protein |
| 48 | 1.3 | 0.552 | 37196_at | X79981 | Hs.76206 | 1003 | cadherin 5, type 2, |
| | | | | | | | VE-cadherin (vascular |
| | | | | | | | epithelium) |
| 49 | 1.29 | 0.552 | 36958_at | X95735 | Hs.75873 | 7791 | zyxin |
| 50 | 1.28 | 0.552 | 38685_at | AL035306 | Hs.106823 | 84295 | hypothetical protein |
| | | | | | | | MGC14797 |
| 51 | 1.28 | 0.551 | 37307_at | X04828 | Hs.77269 | 2771 | guanine nucleotide |
| | | | | | | | binding protein (G |
| | | | | | | | protein), alpha |
| | | | | | | | inhibiting activity |
| | | | | | | | polypeptide 2 |
| 52 | 1.27 | 0.551 | 38704_at | AB007934 | Hs.108258 | 23499 | actin binding protein; |
| | | | | | | | macrophin |
| | | | | | | | (microfilament and |
| | | | | | | | actin filament cross- |
| | | | | | | | linker protein) |
| 53 | 1.27 | 0.551 | 32166_at | AB028950 | Hs.18420 | 7094 | KIAA1027 protein |
| 54 | 1.26 | 0.550 | 34874_at | AJ004832 | Hs.5038 | 10908 | neuropathy target |
| | | | | | | | esterase |
| 55 | 1.26 | 0.549 | 36937_s_at | U90878 | Hs.75807 | 9124 | PDZ and LIM domain |
| | | | | | | | 1 (elfin) |
| 56 | 1.25 | 0.549 | 37247_at | AF047419 | Hs.78061 | 6943 | transcription factor 21 |
| 57 | 1.25 | 0.549 | 39541_at | W52003 | Hs.10491 | 57493 | KIAA1237 protein |
| 58 | 1.25 | 0.547 | 590_at | M32334 | | | intercellular adhesion |
| | | | | | | | molecule 2 |
| 59 | 1.24 | 0.547 | 37168_at | AB013924 | Hs.10887 | 27074 | similar to lysosome- |
| | | | | | | | associated membrane |
| | | | | | | | glycoprotein |
| 60 | 1.23 | 0.547 | 39038_at | AF093118 | Hs.11494 | 10516 | fibulin 5 |
| 61 | 1.23 | 0.547 | 40456_at | AL049963 | Hs.284205 | 64116 | up-regulated by BCG- |
| | | | | | | | CWS |
| 62 | 1.23 | 0.546 | 40202_at | D31716 | Hs.150557 | 687 | basic transcription |
| | | | | | | | element binding |
| | | | | | | | protein 1 |
| 63 | 1.21 | 0.546 | 31856_at | Z24680 | Hs.151641 | 2615 | glycoprotein A |
| | | | | | | | repetitions |
| | | | | | | | predominant |
| 64 | 1.2 | 0.545 | 32321_at | X56841 | Hs.181392 | 3133 | major |
| | | | | | | | histocompatibility |
| | | | | | | | complex, class I, E |
| 65 | 1.19 | 0.545 | 37042_at | U09577 | Hs.76873 | 8692 | hyaluronoglucos- |
| | | | | | | | aminidase 2 |
| 66 | 1.19 | 0.545 | 1897_at | L07594 | Hs.79059 | 7049 | transforming growth |
| | | | | | | | factor, beta receptor III |
| | | | | | | | (betaglycan, 300 kD) |
| 67 | 1.18 | 0.544 | 35783_at | H93123 | Hs.66708 | 9341 | vesicle-associated |
| | | | | | | | membrane protein 3 |
| | | | | | | | (cellubrevin) |
| 68 | 1.17 | 0.544 | 32052_at | L48215 | Hs.155376 | 3043 | hemoglobin, beta |
| 69 | 1.17 | 0.544 | 33862_at | AF017786 | Hs.173717 | 8613 | phosphatidic acid |
| | | | | | | | phosphatase type 2B |
| 70 | 1.16 | 0.543 | 32812_at | AB029025 | Hs.202949 | 22998 | KIAA1102 protein |
| 71 | 1.16 | 0.543 | 36452_at | AB028952 | Hs.5307 | 11346 | synaptopodin |
| 72 | 1.15 | 0.542 | 37407_s_at | AF013570 | Hs.78344 | 4629 | myosin, heavy |
| | | | | | | | polypeptide 11, |
| | | | | | | | smooth muscle |
| 73 | 1.15 | 0.541 | 38406_f_at | AI207842 | Hs.8272 | 5730 | prostaglandin D2 |
| | | | | | | | synthase (21 kD, brain) |
| 74 | 1.14 | 0.541 | 216_at | M98539 | | | prostaglandin D2 |
| | | | | | | | synthase (21 kD, brain) |
| 75 | 1.14 | 0.541 | 38700_at | M33146 | Hs.108080 | 1465 | cysteine and glycine- |
| | | | | | | | rich protein 1 |
| 76 | 1.13 | 0.541 | 39182_at | U87947 | Hs.9999 | 2014 | epithelial membrane |
| | | | | | | | protein 3 |
| 77 | 1.13 | 0.541 | 39315_at | D13628 | Hs.2463 | 284 | angiopoietin 1 |
| 78 | 1.13 | 0.540 | 36207_at | D67029 | Hs.75232 | 6397 | SEC14 (S. cerevisiae)- |
| | | | | | | | like 1 |
| 79 | 1.13 | 0.540 | 38338_at | AI201108 | Hs.9651 | 6237 | related RAS viral (r- |
| | | | | | | | ras) oncogene |
| | | | | | | | homolog |
| 80 | 1.11 | 0.540 | 38691_s_at | J03553 | Hs.1074 | 6440 | surfactant, pulmonary- |
| | | | | | | | associated protein C |
| 81 | 1.11 | 0.539 | 32109_at | AA524547 | Hs.160318 | 5348 | FXYD domain- |
| | | | | | | | containing ion |
| | | | | | | | transport regulator 1 |
| | | | | | | | (phospholemman) |
| 82 | 1.11 | 0.539 | 38044_at | AF035283 | Hs.8022 | 11170 | TU3A protein |
| 83 | 1.1 | 0.537 | 40567_at | X01703 | Hs.272897 | 7846 | Tubulin, alpha, brain- |
| | | | | | | | specific |
| 84 | 1.1 | 0.537 | 36908_at | M93221 | | | mannose receptor, C |
| | | | | | | | typel |
| 85 | 1.1 | 0.537 | 35183_at | U78735 | Hs.26630 | 21 | ATP-binding cassette, |
| | | | | | | | sub-family A (ABC1), |
| | | | | | | | member 3 |
| 86 | 1.09 | 0.537 | 538_at | S53911 | Hs.85289 | 947 | CD34 antigen |
| 87 | 1.09 | 0.536 | 33283_at | AF106941 | Hs.18142 | 409 | arrestin, beta 2 |
| 88 | 1.08 | 0.536 | 33295_at | X85785 | Hs.183 | 2532 | Duffy blood group |
| 89 | 1.08 | 0.536 | 38972_at | AF052169 | Hs.109438 | | clone 24775 |
| 90 | 1.07 | 0.536 | 33137_at | Y13622 | Hs.85087 | 8425 | latent transforming |
| | | | | | | | growth factor beta |
| | | | | | | | binding protein 4 |
| 91 | 1.07 | 0.535 | 39588_at | AF055872 | Hs.26401 | 8742 | tumor necrosis factor |
| | | | | | | | (ligand) superfamily, |
| | | | | | | | member 12 |
| 92 | 1.06 | 0.535 | 38786_at | AL079279 | Hs.8963 | | clone EUROIMAGE |
| | | | | | | | 248114 |
| 93 | 1.06 | 0.535 | 33833_at | J05243 | Hs.77196 | 6709 | spectrin, alpha, non- |
| | | | | | | | erythrocytic 1 (alpha- |
| | | | | | | | fodrin) |
| 94 | 1.06 | 0.534 | 35164_at | AF084481 | Hs.26077 | 7466 | Wolfram syndrome 1 |
| | | | | | | | (wolframin) |
| 95 | 1.05 | 0.534 | 37718_at | D43636 | Hs.79025 | 23182 | KIAA0096 protein |
| 96 | 1.05 | 0.534 | 1780_at | M19722 | Hs.1422 | 2268 | Gardner-Rasheed |
| | | | | | | | feline sarcoma viral |
| | | | | | | | (v-fgr) oncogene |
| | | | | | | | homolog |
| 97 | 1.05 | 0.534 | 36668_at | M28713 | | | diaphorase (NADH) |
| | | | | | | | (cytochrome b-5 |
| | | | | | | | reductase) |
| 98 | 1.05 | 0.534 | 41338_at | AI951946 | Hs.21907 | 11143 | histone |
| | | | | | | | acetyltransferase |
| 99 | 1.04 | 0.533 | 32527_at | AI381790 | Hs.74120 | 10974 | adipose specific 2 |
| 100 | 1.04 | 0.533 | 34363_at | Z11793 | Hs.3314 | 6414 | selenoprotein P, |
| | | | | | | | plasma, 1 |
| 101 | 1.04 | 0.533 | 37743_at | U60060 | Hs.79226 | 9638 | fasciculation and |
| | | | | | | | elongation protein zeta |
| | | | | | | | 1 (zygin I) |
| 102 | 1.03 | 0.533 | 32838_at | S67247 | Hs.296842 | | smooth muscle myosin |
| | | | | | | | heavy chain isoform |
| | | | | | | | SMemb [human, |
| | | | | | | | umbilical cord, fetal |
| | | | | | | | aorta, |
| 103 | 1.03 | 0.533 | 40739_at | M83670 | Hs.89485 | 762 | carbonic anhydrase IV |
| 104 | 1.03 | 0.533 | 39057_at | L04733 | Hs.117977 | 3831 | kinesin 2 (60-70 kD) |
| 105 | 1.03 | 0.532 | 35625_at | X94630 | Hs.3107 | 976 | CD97 antigen |
| 106 | 1.03 | 0.531 | 40742_at | M16591 | Hs.89555 | 3055 | hemopoietic cell |
| | | | | | | | kinase |
| 107 | 1.03 | 0.531 | 38717_at | AL050159 | Hs.288771 | 25840 | DKFZP586A0522 |
| | | | | | | | protein |
| 108 | 1.03 | 0.531 | 32254_at | AL050223 | Hs.194534 | 6844 | vesicle-associated |
| | | | | | | | membrane protein 2 |
| | | | | | | | (synaptobrevin 2) |
| 109 | 1.03 | 0.531 | 38026_at | U01244 | Hs.79732 | 2192 | fibulin 1 |
| 110 | 1.02 | 0.530 | 37958_at | AL049257 | Hs.8769 | 83604 | hypothetical protein |
| | | | | | | | DKFZp761J17121 |
| 111 | 1.02 | 0.530 | 37598_at | D79990 | Hs.80905 | 9770 | Ras association |
| | | | | | | | (RalGDS/AF-6) |
| | | | | | | | domain family 2 |
| 112 | 1.02 | 0.530 | 39145_at | J02854 | Hs.9615 | 10398 | myosin regulatory |
| | | | | | | | light chain 2, smooth |
| | | | | | | | muscle isoform |
| 113 | 1.02 | 0.530 | 40775_at | AL021786 | Hs.17109 | 9452 | integral membrane |
| | | | | | | | protein 2A |
| 114 | 1.02 | 0.529 | 35282_r_at | M33680 | Hs.54457 | 975 | CD81 antigen (target |
| | | | | | | | of antiproliferative |
| | | | | | | | antibody 1) |
| 115 | 1.02 | 0.529 | 37023_at | J02923 | Hs.76506 | 3936 | lymphocyte cytosolic |
| | | | | | | | protein 1 (L-plastin) |
| 116 | 1.02 | 0.529 | 38748_at | U76421 | Hs.85302 | 104 | adenosine deaminase, |
| | | | | | | | RNA-specific, B1 |
| | | | | | | | (homolog of rat |
| | | | | | | | RED1) |
| 117 | 1.01 | 0.529 | 41198_at | AF055008 | Hs.180577 | 2896 | granulin |
| 118 | 1 | 0.528 | 34194_at | AL049313 | Hs.21103 | | clone DKFZp564B076 |
| 119 | 1 | 0.528 | 33158_at | M97252 | Hs.89591 | 3730 | Kallmann syndrome 1 |
| | | | | | | | sequence |
| 120 | 0.99 | 0.528 | 31525_s_at | J00153 | | | hemoglobin, alpha 2 |
| 121 | 0.99 | 0.527 | 32847_at | U48959 | Hs.211582 | 4638 | myosin, light |
| | | | | | | | polypeptide kinase |
| 122 | 0.98 | 0.527 | 38110_at | AF000652 | Hs.8180 | 6386 | syndecan binding |
| | | | | | | | protein (syntenin) |
| 123 | 0.98 | 0.527 | 39220_at | T92248 | Hs.2240 | 7356 | uteroglobin |
| 124 | 0.98 | 0.527 | 38119_at | X12496 | Hs.81994 | 2995 | glycophorin C |
| | | | | | | | (Gerbich blood group) |
| 125 | 0.98 | 0.527 | 40936_at | AI651806 | Hs.19280 | 51232 | cysteine-rich motor |
| | | | | | | | neuron 1 |
| 126 | 0.98 | 0.527 | 37194_at | M68891 | Hs.334695 | 2624 | GATA-binding protein |
| | | | | | | | 2 |
| 127 | 0.97 | 0.526 | 41620_at | AB018259 | Hs.118140 | 9732 | KIAA0716 gene |
| | | | | | | | product |
| 128 | 0.96 | 0.526 | 37951_at | AF035119 | Hs.8700 | 10395 | deleted in liver cancer |
| | | | | | | | 1 |
| 129 | 0.95 | 0.526 | 657_at | L11373 | Hs.284180 | 5098 | protocadherin gamma |
| | | | | | | | subfamily C, 3 |
| 130 | 0.95 | 0.525 | 37009_at | AL035079 | Hs.76359 | 847 | catalase |
| 131 | 0.95 | 0.525 | 33390_at | AA203487 | Hs.314363 | | CD68 |
| 132 | 0.95 | 0.525 | 40434_at | U97519 | Hs.16426 | 5420 | podocalyxin-like |
| 133 | 0.95 | 0.525 | 37022_at | U41344 | | | proline arginine-rich |
| | | | | | | | end leucine-rich repeat |
| | | | | | | | protein |
| 134 | 0.95 | 0.525 | 31792_at | M20560 | Hs.1378 | 306 | annexin A3 |
| 135 | 0.94 | 0.524 | 38113_at | AB018339 | Hs.8182 | 23345 | synaptic nuclei |
| | | | | | | | expressed gene 1b |
| 136 | 0.94 | 0.524 | 35152_at | AJ001016 | Hs.25691 | 10268 | receptor (calcitonin) |
| | | | | | | | activity modifying |
| | | | | | | | protein 3 |
| 137 | 0.93 | 0.524 | 1879_at | M14949 | | | related RAS viral (r- |
| | | | | | | | ras) oncogene |
| | | | | | | | homolog |
| 138 | 0.93 | 0.524 | 41734_at | AB020677 | Hs.18166 | 22898 | KIAA0870 protein |
| 139 | 0.92 | 0.524 | 36495_at | U21931 | | | fructose-1,6- |
| | | | | | | | bisphosphatase 1 |
| 140 | 0.92 | 0.524 | 1370_at | M29696 | Hs.237868 | 3575 | interleukin 7 receptor |
| 141 | 0.92 | 0.523 | 1598_g_at | L13720 | Hs.78501 | 2621 | growth arrest-specific |
| | | | | | | | 6 |
| 142 | 0.92 | 0.523 | 38363_at | W60864 | Hs.9963 | 7305 | TYRO protein tyrosine |
| | | | | | | | kinase binding protein |
| 143 | 0.92 | 0.523 | 32035_at | M16942 | Hs.318720 | | MHC class II HLA- |
| | | | | | | | DRw53-associated |
| | | | | | | | glycoprotein beta- |
| | | | | | | | chain |
| 144 | 0.92 | 0.523 | 41209_at | M15856 | Hs.180878 | 4023 | lipoprotein lipase |
| 145 | 0.92 | 0.523 | 1612_s_at | X56681 | Hs.2780 | 3727 | jun D proto-oncogene |
| 146 | 0.91 | 0.523 | 34091_s_at | Z19554 | Hs.297753 | 7431 | vimentin |
| 147 | 0.91 | 0.522 | 479_at | U53446 | Hs.81988 | 1601 | disabled (Drosophila) |
| | | | | | | | homolog 2 (mitogen- |
| | | | | | | | responsive |
| | | | | | | | phosphoprotein) |
| 148 | 0.91 | 0.522 | 39615_at | AB028949 | Hs.27742 | 23254 | KIAA1026 protein |
| 149 | 0.9 | 0.522 | 692_s_at | J02947 | Hs.2420 | 6649 | superoxide dismutase |
| | | | | | | | 3, extracellular |
| 150 | 0.9 | 0.521 | 36065_at | AF052389 | Hs.4980 | 9079 | LIM domain binding 2 |
| 151 | 0.9 | 0.521 | 40570_at | AF032885 | Hs.170133 | 2308 | forkhead box O1A |
| | | | | | | | (rhabdomyosarcoma) |
| 152 | 0.9 | 0.521 | 37148_at | AF025533 | Hs.105928 | 11025 | leukocyte |
| | | | | | | | immunoglobulin-like |
| | | | | | | | receptor, subfamily B |
| | | | | | | | (with TM and ITIM |
| | | | | | | | domains), member 3 |
| 153 | 0.89 | 0.521 | 41288_at | AL036744 | Hs.279009 | 4256 | matrix Gla protein |
| 154 | 0.89 | 0.521 | 32811_at | X98507 | Hs.286226 | 4641 | myosin IB |
| 155 | 0.88 | 0.521 | 37384_at | D13640 | Hs.278441 | 9647 | KIAA0015 gene |
| | | | | | | | product |
| 156 | 0.88 | 0.520 | 41325_at | AF006823 | Hs.24040 | 3777 | potassium channel, |
| | | | | | | | subfamily K, member |
| | | | | | | | 3 (TASK) |
| 157 | 0.88 | 0.520 | 40322_at | D12763 | Hs.66 | 9173 | interleukin 1 receptor- |
| | | | | | | | like 1 |
| 158 | 0.88 | 0.520 | 32905_s_at | M30038 | Hs.334455 | 7176 | tryptase, alpha |
| 159 | 0.87 | 0.520 | 34873_at | Y16241 | Hs.5025 | 10529 | nebulette |
| 160 | 0.87 | 0.520 | 610_at | M15169 | Hs.2551 | 154 | adrenergic, beta-2-, |
| | | | | | | | receptor, surface |
| 161 | 0.87 | 0.520 | 41644_at | AB018333 | Hs.12002 | 23328 | KIAA0790 protein |
| 162 | 0.87 | 0.520 | 36894_at | AL031846 | | | chromobox homolog 7 |
| 163 | 0.87 | 0.520 | 33891_at | AL080061 | Hs.25035 | 25932 | chloride intracellular |
| | | | | | | | channel 4 |
| 164 | 0.87 | 0.520 | 40147_at | U18009 | Hs.157236 | 10493 | membrane protein of |
| | | | | | | | cholinergic synaptic |
| | | | | | | | vesicles |
| 165 | 0.87 | 0.520 | 38796_at | X03084 | Hs.8986 | 713 | complement |
| | | | | | | | component 1, q |
| | | | | | | | subcomponent, beta |
| | | | | | | | polypeptide |
| 166 | 0.87 | 0.520 | 36856_at | W28743 | Hs.7159 | 80301 | hypothetical protein |
| | | | | | | | PP1628 |
| 167 | 0.87 | 0.520 | 1038_s_at | U19247 | | | interferon gamma |
| | | | | | | | receptor 1 |
| 168 | 0.86 | 0.519 | 34637_f_at | M12963 | Hs.73843 | 124 | alcohol dehydrogenase |
| | | | | | | | 1 (class I), alpha |
| | | | | | | | polypeptide |
| 169 | 0.85 | 0.519 | 38747_at | M81945 | | | CD34 antigen |
| 170 | 0.84 | 0.519 | 32747_at | X05409 | Hs.195432 | 217 | aldehyde |
| | | | | | | | dehydrogenase 2, |
| | | | | | | | mitochondrial |
| 171 | 0.84 | 0.519 | 32749_s_at | AL050396 | Hs.195464 | 2316 | filamin A, alpha |
| | | | | | | | (actin-binding protein- |
| | | | | | | | 280) |
| 172 | 0.84 | 0.519 | 38087_s_at | W72186 | Hs.81256 | 6275 | S100 calcium-binding |
| | | | | | | | protein A4 (calcium |
| | | | | | | | protein, calvasculin, |
| | | | | | | | metastasin, murine |
| | | | | | | | placental homolog) |
| 173 | 0.84 | 0.518 | 38095_i_at | M83664 | Hs.814 | 3115 | major |
| | | | | | | | histocompatibility |
| | | | | | | | complex, class II, DP |
| | | | | | | | beta 1 |
| 174 | 0.84 | 0.518 | 40203_at | AJ012375 | Hs.150580 | 10209 | putative translation |
| | | | | | | | initiation factor |
| 175 | 0.84 | 0.518 | 34224_at | AC004770 | Hs.21765 | 3995 | flap structure-specific |
| | | | | | | | endonuclease 1 |
| 176 | 0.83 | 0.518 | 307_at | J03600 | Hs.89499 | 240 | arachidonate 5- |
| | | | | | | | lipoxygenase |
| 177 | 0.83 | 0.518 | 38968_at | AB005047 | Hs.109150 | 9467 | SH3-domain binding |
| | | | | | | | protein 5 (BTK- |
| | | | | | | | associated) |
| 178 | 0.83 | 0.517 | 39114_at | AB022718 | Hs.93675 | 11067 | decidual protein |
| | | | | | | | induced by |
| | | | | | | | progesterone |
| 179 | 0.83 | 0.517 | 41385_at | AB023204 | Hs.103839 | 23136 | differentially |
| | | | | | | | expressed in |
| | | | | | | | adenocarcinoma of the |
| | | | | | | | lung |
| 180 | 0.83 | 0.517 | 39400_at | AB028978 | Hs.126084 | 23102 | KIAA1055 protein |
| 181 | 0.83 | 0.517 | 39081_at | AI547258 | Hs.118786 | 4502 | metallothionein 2A |
| 182 | 0.82 | 0.517 | 33813_at | AI813532 | Hs.256278 | 7133 | tumor necrosis factor |
| | | | | | | | receptor superfamily, |
| | | | | | | | member 1B |
| 183 | 0.82 | 0.517 | 31775_at | X65018 | | | surfactant, pulmonary- |
| | | | | | | | associated protein D |
| 184 | 0.82 | 0.517 | 32855_at | L00352 | | | low density lipoprotein |
| | | | | | | | receptor (familial |
| | | | | | | | hypercholesterolemia) |
| 185 | 0.82 | 0.516 | 40480_s_at | M14333 | Hs.169370 | 2534 | FYN oncogene related |
| | | | | | | | to SRC, FOR, YES |
| 186 | 0.81 | 0.516 | 36156_at | U41518 | Hs.74602 | 358 | aquaporin 1 (channel- |
| | | | | | | | forming integral |
| | | | | | | | protein, 28 kD) |
| 187 | 0.81 | 0.516 | 41439_at | AJ001381 | Hs.121576 | | incomplete cDNA for |
| | | | | | | | a mutated allele of a |
| | | | | | | | myosin class I, myh-1c |
| 188 | 0.81 | 0.516 | 774_g_at | D10667 | | | myosin, heavy |
| | | | | | | | polypeptide 11, |
| | | | | | | | smooth muscle |
| 189 | 0.81 | 0.516 | 924_s_at | J03805 | Hs.80350 | 5516 | protein phosphatase 2 |
| | | | | | | | (formerly 2A), |
| | | | | | | | catalytic subunit, beta |
| | | | | | | | isoform |
| 190 | 0.81 | 0.516 | 40771_at | Z98946 | Hs.170328 | 4478 | moesin |
| 191 | 0.81 | 0.515 | 38833_at | X00457 | Hs.914 | | SB classII |
| | | | | | | | histocompatibility |
| | | | | | | | antigen alpha-chain |
| 192 | 0.81 | 0.515 | 41143_at | U12022 | | | calmodulin 1 |
| | | | | | | | (phosphorylase kinase, |
| | | | | | | | delta) |
| 193 | 0.8 | 0.515 | 37176_at | U96078 | Hs.75619 | 3373 | hyaluronoglucos- |
| | | | | | | | aminidase 1 |
| 194 | 0.8 | 0.515 | 36447_at | S80990 | | | ficolin |
| | | | | | | | (collagen/fibrinogen |
| | | | | | | | domain-containing) 1 |
| 195 | 0.8 | 0.515 | 1052_s_at | M83667 | Hs.76722 | 1052 | CCAAT/enhancer |
| | | | | | | | binding protein |
| | | | | | | | (C/EBP), delta |
| 196 | 0.8 | 0.515 | 41723_s_at | M32578 | Hs.180255 | 3123 | major |
| | | | | | | | histocompatibility |
| | | | | | | | complex, class II, DR |
| | | | | | | | beta 1 |
| 197 | 0.8 | 0.515 | 38404_at | M55153 | Hs.8265 | 7052 | transglutaminase 2 (C |
| | | | | | | | polypeptide, protein- |
| | | | | | | | glutamine-gamma- |
| | | | | | | | glutamyltransferase) |
| 198 | 0.8 | 0.515 | 34760_at | D14664 | Hs.2441 | 9936 | KIAA0022 gene |
| | | | | | | | product |
| 199 | 0.79 | 0.515 | 32569_at | L13385 | Hs.77318 | 5048 | platelet-activating |
| | | | | | | | factor acetylhydrolase, |
| | | | | | | | isoform Ib, alpha |
| | | | | | | | subunit (45 kD) |
| 200 | 0.79 | 0.514 | 505_at | U43077 | Hs.160958 | 11140 | CDC37 (cell division |
| | | | | | | | cycle 37, S. cerevisiae, |
| | | | | | | | homolog) |

According to the invention, preferred markers are markers 1-30, preferably 1-20, and more preferably 1-10. Highly preferred markers are transforming growth factor beta receptor II, dihydropyrimidinase-like 2, and tetranectin.

TABLE 6
Colorectal Matastasis Markers
Class: Colon

| | | | | | UNIGENE | | |
|---|---|---|---|---|---|---|---|
| | | | | | (as of | | Desc |
| | | Perm | | GB/TIGR | summer | | (unigene/locuslink |
| | s2n_obs | 0.1% | non_norm_list | Identifier | 2001) | LL_num | or affy) |
| 1 | 2.33 | 0.914 | 40392_at | U51096 | Hs.77399 | 1045 | caudal type homeo |
| | | | | | | | box transcription |
| | | | | | | | factor 2 |
| 2 | 1.58 | 0.728 | 40736_at | X83228 | Hs.89436 | 1015 | cadherin 17, LI |
| | | | | | | | cadherin (liver- |
| | | | | | | | intestine) |
| 3 | 1.55 | 0.719 | 37124_i_at | J04813 | Hs.104117 | 1577 | cytochrome P450, |
| | | | | | | | subfamily IIIA |
| | | | | | | | (niphedipine |
| | | | | | | | oxidase), |
| | | | | | | | polypeptide 5 |
| 4 | 1.52 | 0.715 | 169_at | U51095 | Hs.1545 | 1044 | caudal type homeo |
| | | | | | | | box transcription |
| | | | | | | | factor 1 |
| 5 | 1.45 | 0.701 | 40043_at | X71345 | Hs.58247 | 5647 | protease, serine, 4 |
| | | | | | | | (trypsin 4, brain) |
| 6 | 1.4 | 0.698 | 35644 at | AB014598 | Hs.31720 | 9843 | hephaestin |
| 7 | 1.37 | 0.688 | 38586_at | M10050 | Hs.5241 | 2168 | fatty acid binding |
| | | | | | | | protein 1, liver |
| 8 | 1.37 | 0.682 | 32972_at | Z83819 | Hs.132370 | 27035 | NADPH oxidase 1 |
| 9 | 1.34 | 0.679 | 39951_at | L20826 | Hs.430 | 5357 | plastin 1 (I isoform) |
| 10 | 1.3 | 0.677 | 1229_at | U78556 | Hs.166066 | 10903 | cisplatin resistance |
| | | | | | | | associated |
| 11 | 1.3 | 0.677 | 988_at | X16354 | Hs.50964 | 634 | carcinoembryonic |
| | | | | | | | antigen-related cell |
| | | | | | | | adhesion molecule |
| | | | | | | | 1 (biliary |
| | | | | | | | glycoprotein) |
| 12 | 1.3 | 0.669 | 37415_at | AB018258 | Hs.109358 | 23120 | ATPase, Class V, |
| | | | | | | | type 10B |
| 13 | 1.25 | 0.668 | 41708_at | AB028957 | Hs.12896 | 23314 | KIAA1034 protein |
| 14 | 1.22 | 0.656 | 765_s_at | AB006781 | Hs.5302 | 3960 | lectin, galactoside- |
| | | | | | | | binding, soluble, 4 |
| | | | | | | | (galectin 4) |
| 15 | 1.21 | 0.654 | 39697_at | U26726 | Hs.1376 | 3291 | hydroxysteroid (11- |
| | | | | | | | beta) |
| | | | | | | | dehydrogenase 2 |
| 16 | 1.2 | 0.650 | 33559_at | U61412 | | | PTK6 protein |
| | | | | | | | tyrosine kinase 6 |
| 17 | 1.2 | 0.649 | 33904_at | AB000714 | Hs.25640 | 1365 | claudin 3 |
| 18 | 1.19 | 0.649 | 41266_at | X53586 | Hs.227730 | 3655 | integrin, alpha 6 |
| 19 | 1.19 | 0.648 | 36170_at | D83198 | Hs.7486 | 23474 | protein expressed in |
| | | | | | | | thyroid |
| 20 | 1.18 | 0.648 | 37847_at | AB006955 | Hs.132945 | 10083 | PDZ-73 protein |
| 21 | 1.16 | 0.646 | 34595_at | AF105424 | Hs.5394 | 4640 | myosin, heavy |
| | | | | | | | polypeptide-like |
| | | | | | | | (110 kD) |
| 22 | 1.16 | 0.644 | 40694_at | X73502 | Hs.84905 | 54474 | cytokeratin 20 |
| 23 | 1.14 | 0.639 | 35415_at | X12901 | Hs.166068 | 7429 | villin 1 |
| 24 | 1.14 | 0.638 | 899_at | L38517 | Hs.69351 | 3549 | Indian hedgehog |
| | | | | | | | (Drosophila) |
| | | | | | | | homolog |
| 25 | 1.11 | 0.638 | 37875_at | U79725 | Hs.143131 | 10223 | glycoprotein A33 |
| | | | | | | | (transmembrane) |
| 26 | 1.11 | 0.635 | 41678_at | AF025304 | Hs.125124 | 2048 | EphB2 |
| 27 | 1.1 | 0.632 | 32649_at | X59871 | Hs.169294 | 6932 | transcription factor |
| | | | | | | | 7 (T-cell specific, |
| | | | | | | | HMG-box) |
| 28 | 1.08 | 0.629 | 35114_at | AF084645 | Hs.118138 | 8856 | nuclear receptor |
| | | | | | | | subfamily 1, group |
| | | | | | | | I, member 2 |
| 29 | 1.07 | 0.629 | 36832_at | AB015630 | Hs.69009 | 10331 | transmembrane |
| | | | | | | | protein 3 |
| 30 | 1.07 | 0.627 | 41396 at | AB006629 | Hs.104717 | 7461 | cytoplasmic linker 2 |
| 31 | 1.07 | 0.624 | 35256_at | AL096737 | Hs.5167 | | clone |
| | | | | | | | DKFZp434F152 |
| 32 | 1.07 | 0.620 | 33436_at | Z46629 | Hs.2316 | 6662 | SRY (sex |
| | | | | | | | determining region |
| | | | | | | | Y)-box 9 |
| | | | | | | | (campomelic |
| | | | | | | | dysplasia, |
| | | | | | | | autosomal sex- |
| | | | | | | | reversal) |
| 33 | 1.05 | 0.620 | 33789_at | AF088219 | Hs.272493 | 6359 | small inducible |
| | | | | | | | cytokine subfamily |
| | | | | | | | A (Cys-Cys), |
| | | | | | | | member 23 |
| 34 | 1.05 | 0.619 | 34450_at | M73489 | Hs.1085 | 2984 | guanylate cyclase |
| | | | | | | | 2C (heat stable |
| | | | | | | | enterotoxin |
| | | | | | | | receptor) |
| 35 | 1.04 | 0.619 | 31355_at | U77629 | Hs.135639 | 430 | achaete-scute |
| | | | | | | | complex |
| | | | | | | | (Drosophila) |
| | | | | | | | homolog-like 2 |
| 36 | 1.03 | 0.618 | 39732_at | X73882 | Hs.146388 | 9053 | microtubule- |
| | | | | | | | associated protein 7 |
| 37 | 1.03 | 0.617 | 40061_at | D83784 | Hs.154104 | 5326 | pleiomorphic |
| | | | | | | | adenoma gene-like |
| | | | | | | | 2 |
| 38 | 1.03 | 0.617 | 38469_at | M35252 | Hs.84072 | 7103 | transmembrane 4 |
| | | | | | | | superfamily |
| | | | | | | | member 3 |
| 39 | 1.03 | 0.615 | 246_at | M25629 | Hs.123107 | 3816 | kallikrein 1, |
| | | | | | | | renal/pancreas/salivary |
| 40 | 1.03 | 0.613 | 36742_at | U34249 | Hs.337461 | 89870 | ring finger protein 9 |
| 41 | 1.02 | 0.613 | 36816_s_at | M28668 | Hs.663 | 1080 | cystic fibrosis |
| | | | | | | | transmembrane |
| | | | | | | | conductance |
| | | | | | | | regulator, ATP- |
| | | | | | | | binding cassette |
| | | | | | | | (sub-family C, |
| | | | | | | | member 7) |
| 42 | 1.01 | 0.612 | 38495_s_at | U27328 | Hs.169238 | 2525 | fucosyltransferase 3 |
| | | | | | | | (galactoside 3(4)-L- |
| | | | | | | | fucosyltransferase, |
| | | | | | | | Lewis blood group |
| | | | | | | | included) |
| 43 | 1.01 | 0.611 | 1973_s_at | V00568 | Hs.79070 | 4609 | v-myc avian |
| | | | | | | | myelocytomatosis |
| | | | | | | | viral oncogene |
| | | | | | | | homolog |
| 44 | 1.01 | 0.611 | 37857_at | AL080188 | Hs.137556 | 92211 | MT-protocadherin |
| 45 | 1 | 0.610 | 40198_at | L06132 | Hs.149155 | 7416 | voltage-dependent |
| | | | | | | | anion channel 1 |
| 46 | 0.99 | 0.607 | 33824_at | X74929 | Hs.242463 | 3856 | keratin 8 |
| 47 | 0.99 | 0.607 | 38160_at | AF011333 | Hs.153563 | 4065 | lymphocyte antigen |
| | | | | | | | 75 |
| 48 | 0.99 | 0.607 | 34280_at | Y09765 | Hs.22785 | 2564 | gamma- |
| | | | | | | | aminobutyric acid |
| | | | | | | | (GABA) A |
| | | | | | | | receptor, epsilon |
| 49 | 0.98 | 0.606 | 31608_g_at | AJ002428 | Hs.201553 | 10065 | voltage-dependent |
| | | | | | | | anion channel 1 |
| | | | | | | | pseudogene |
| 50 | 0.98 | 0.606 | 820_at | U77604 | Hs.81874 | 4258 | microsomal |
| | | | | | | | glutathione S- |
| | | | | | | | transferase 2 |
| 51 | 0.98 | 0.606 | 34176_at | AF091087 | Hs.206501 | 57228 | hypothetical protein |
| | | | | | | | from clone 643 |
| 52 | 0.98 | 0.605 | 40647_at | Z32684 | Hs.78919 | 7504 | Kell blood group |
| | | | | | | | precursor (McLeod |
| | | | | | | | phenotype) |
| 53 | 0.98 | 0.604 | 36655_at | L27476 | Hs.75608 | 9414 | tight junction |
| | | | | | | | protein 2 (zona |
| | | | | | | | occludens 2) |
| 54 | 0.97 | 0.604 | 37050_r_at | AI130910 | Hs.76927 | 10953 | translocase of outer |
| | | | | | | | mitochondrial |
| | | | | | | | membrane 34 |
| 55 | 0.97 | 0.604 | 32324_at | X57346 | Hs.279920 | 7529 | tyrosine 3- |
| | | | | | | | monooxygenase/try |
| | | | | | | | ptophan 5- |
| | | | | | | | monooxygenase |
| | | | | | | | activation protein, |
| | | | | | | | beta polypeptide |
| 56 | 0.96 | 0.604 | 41715_at | Y11312 | Hs.132463 | 5287 | phosphoinositide-3- |
| | | | | | | | kinase, class 2, beta |
| | | | | | | | polypeptide |
| 57 | 0.96 | 0.604 | 40492_at | AB020633 | Hs.169600 | 23045 | KIAA0826 protein |
| 58 | 0.96 | 0.603 | 575_s_at | M93036 | | | tumor-associated |
| | | | | | | | calcium signal |
| | | | | | | | transducer 1 |
| 59 | 0.95 | 0.603 | 1756_f_at | D00003 | Hs.329704 | 1575 | cytochrome P450, |
| | | | | | | | subfamily IIIA |
| | | | | | | | (niphedipine |
| | | | | | | | oxidase), |
| | | | | | | | polypeptide 3 |
| 60 | 0.95 | 0.603 | 37950_at | X74496 | Hs.86978 | 5550 | prolyl |
| | | | | | | | endopeptidase |
| 61 | 0.95 | 0.603 | 35489_at | M82962 | Hs.179704 | 4224 | meprin A, alpha |
| | | | | | | | (PABA peptide |
| | | | | | | | hydrolase) |
| 62 | 0.95 | 0.603 | 39721_at | U09303 | Hs.144700 | 1947 | ephrin-B1 |
| 63 | 0.94 | 0.602 | 34803_at | AF022789 | Hs.42400 | 9959 | ubiquitin specific |
| | | | | | | | protease 12 |
| 64 | 0.94 | 0.602 | 32587_at | U07802 | Hs.78909 | 678 | butyrate response |
| | | | | | | | factor 2 (EGF- |
| | | | | | | | response factor 2) |
| 65 | 0.94 | 0.602 | 41359_at | Z98265 | Hs.26557 | 11187 | plakophilin 3 |
| 66 | 0.93 | 0.602 | 1291_s_at | L03840 | Hs.165950 | 2264 | fibroblast growth |
| | | | | | | | factor receptor 4 |
| 67 | 0.93 | 0.602 | 37253_at | X92493 | Hs.78406 | 8395 | phosphatidylinositol- |
| | | | | | | | 4-phosphate 5- |
| | | | | | | | kinase, type I, beta |
| 68 | 0.92 | 0.601 | 38005_at | AJ005866 | Hs.90078 | 11046 | nucleotide-sugar |
| | | | | | | | transporter similar |
| | | | | | | | to C. elegans sqv-7 |
| 69 | 0.92 | 0.601 | 41448_at | AC004080 | Hs.110637 | 3206 | even-skipped |
| | | | | | | | homeo box 1 |
| | | | | | | | (homolog of |
| | | | | | | | Drosophila) |
| 70 | 0.91 | 0.600 | 39748_at | AL050021 | Hs.14846 | | clone |
| | | | | | | | DKFZp564D016 |
| 71 | 0.91 | 0.600 | 35276_at | AB000712 | Hs.5372 | 1364 | claudin 4 |
| 72 | 0.9 | 0.599 | 37244_at | AA746355 | Hs.77917 | 7347 | ubiquitin carboxyl- |
| | | | | | | | terminal esterase L3 |
| | | | | | | | (ubiquitin |
| | | | | | | | thiolesterase) |
| 73 | 0.9 | 0.599 | 41530_at | D16294 | Hs.32500 | 10449 | acetyl-Coenzyme A |
| | | | | | | | acyltransferase 2 |
| | | | | | | | (mitochondrial 3- |
| | | | | | | | oxoacyl-Coenzyme |
| | | | | | | | A thiolase) |
| 74 | 0.9 | 0.598 | 36289_f_at | U27333 | Hs.32956 | 2528 | fucosyltransferase 6 |
| | | | | | | | (alpha (1,3) |
| | | | | | | | fucosyltransferase) |
| 75 | 0.9 | 0.598 | 36846_s_at | AA121509 | Hs.70830 | 51690 | U6 snRNA- |
| | | | | | | | associated Sm-like |
| | | | | | | | protein LSm7 |
| 76 | 0.89 | 0.597 | 35262_at | AF022229 | Hs.5215 | 3692 | integrin beta 4 |
| | | | | | | | binding protein |
| 77 | 0.89 | 0.597 | 41816_at | AL049851 | Hs.57973 | 29775 | hypothetical protein |
| 78 | 0.89 | 0.597 | 38739_at | AF017257 | Hs.85146 | 2114 | v-ets avian |
| | | | | | | | erythroblastosis |
| | | | | | | | virus E26 oncogene |
| | | | | | | | homolog 2 |
| 79 | 0.89 | 0.596 | 1936_s_at | HG3523- | | | Proto-Oncogene C- |
| | | | | HT4899 | | | Myc, Alt. Splice 3, |
| | | | | | | | Orf 114 |
| 80 | 0.89 | 0.596 | 31948_at | X79563 | Hs.1948 | 6227 | ribosomal protein |
| | | | | | | | S21 |
| 81 | 0.88 | 0.596 | 36687_at | N50520 | Hs.75752 | 1349 | cytochrome c |
| | | | | | | | oxidase subunit |
| | | | | | | | VIIb |
| 82 | 0.88 | 0.595 | 2042_s_at | M15024 | Hs.1334 | 4602 | v-myb avian |
| | | | | | | | myeloblastosis viral |
| | | | | | | | oncogene homolog |
| 83 | 0.87 | 0.595 | 38375_at | AF112219 | Hs.82193 | 2098 | esterase |
| | | | | | | | D/formylglutathione |
| | | | | | | | hydrolase |
| 84 | 0.86 | 0.594 | 35961_at | AL049390 | Hs.22689 | | clone |
| | | | | | | | DKFZp586O1318 |
| 85 | 0.86 | 0.594 | 1582_at | M29540 | Hs.220529 | 1048 | carcinoembryonic |
| | | | | | | | antigen-related cell |
| | | | | | | | adhesion molecule 5 |
| 86 | 0.86 | 0.594 | 37888_at | D87449 | Hs.82635 | 23169 | KIAA0260 protein |
| 87 | 0.86 | 0.594 | 266_s_at | L33930 | Hs.286124 | 934 | CD24 antigen |
| | | | | | | | (small cell lung |
| | | | | | | | carcinoma cluster 4 |
| | | | | | | | antigen) |
| 88 | 0.86 | 0.593 | 31845_at | U32645 | Hs.151139 | 2000 | E74-like factor 4 |
| | | | | | | | (ets domain |
| | | | | | | | transcription factor) |
| 89 | 0.86 | 0.593 | 37211_at | M93107 | Hs.76893 | 622 | 3-hydroxybutyrate |
| | | | | | | | dehydrogenase |
| | | | | | | | (heart, |
| | | | | | | | mitochondrial) |
| 90 | 0.86 | 0.592 | 35345_at | X83618 | Hs.59889 | 3158 | 3-hydroxy-3- |
| | | | | | | | methylglutaryl- |
| | | | | | | | Coenzyme A |
| | | | | | | | synthase 2 |
| | | | | | | | (mitochondrial) |
| 91 | 0.86 | 0.592 | 41236_at | U79252 | Hs.240062 | 29787 | hypothetical protein |
| 92 | 0.86 | 0.592 | 37698_at | X97335 | Hs.78921 | 8165 | A kinase (PRKA) |
| | | | | | | | anchor protein 1 |
| 93 | 0.85 | 0.591 | 32585_at | AF027299 | Hs.7857 | 2037 | erythrocyte |
| | | | | | | | membrane protein |
| | | | | | | | band 4.1-like 2 |
| 94 | 0.85 | 0.590 | 38808_at | D64154 | Hs.90107 | 11047 | cell membrane |
| | | | | | | | glycoprotein, |
| | | | | | | | 110000M (r) |
| | | | | | | | (surface antigen) |
| 95 | 0.85 | 0.590 | 37104_at | L40904 | Hs.100724 | 5468 | peroxisome |
| | | | | | | | proliferative |
| | | | | | | | activated receptor, |
| | | | | | | | gamma |
| 96 | 0.85 | 0.590 | 1317_at | X70040 | Hs.2942 | 4486 | macrophage |
| | | | | | | | stimulating 1 |
| | | | | | | | receptor (c-met- |
| | | | | | | | related tyrosine |
| | | | | | | | kinase) |
| 97 | 0.84 | 0.590 | 37413_at | J05257 | Hs.109 | 1800 | dipeptidase 1 |
| | | | | | | | (renal) |
| 98 | 0.84 | 0.589 | 36345_g_at | U34038 | Hs.154299 | 2150 | coagulation factor II |
| | | | | | | | (thrombin) |
| | | | | | | | receptor-like 1 |
| 99 | 0.84 | 0.589 | 38036_at | L35035 | Hs.79886 | 22934 | ribose 5-phosphate |
| | | | | | | | isomerase A (ribose |
| | | | | | | | 5-phosphate |
| | | | | | | | epimerase) |
| 100 | 0.84 | 0.589 | 39765_at | AB002318 | Hs.150443 | 23079 | KIAA0320 protein |
| 101 | 0.84 | 0.588 | 36363_at | U30930 | Hs.158540 | 7368 | UDP |
| | | | | | | | glycosyltransferase |
| | | | | | | | 8 (UDP-galactose |
| | | | | | | | ceramide |
| | | | | | | | galactosyltransferase) |
| 102 | 0.84 | 0.587 | 1031_at | U09564 | Hs.75761 | 6732 | SFRS protein |
| | | | | | | | kinase 1 |
| 103 | 0.84 | 0.587 | 35913_at | U88047 | Hs.198515 | 1820 | dead ringer |
| | | | | | | | (Drosophila)-like 1 |
| 104 | 0.83 | 0.587 | 39119_s_at | AA631972 | Hs.943 | 9235 | natural killer cell |
| | | | | | | | transcript 4 |
| 105 | 0.83 | 0.587 | 37896_at | AI474125 | Hs.82961 | 7033 | trefoil factor 3 |
| | | | | | | | (intestinal) |
| 106 | 0.83 | 0.587 | 33892_at | X97675 | Hs.25051 | 5318 | plakophilin 2 |
| 107 | 0.83 | 0.587 | 1506_at | D11086 | Hs.84 | 3561 | interleukin 2 |
| | | | | | | | receptor, gamma |
| | | | | | | | (severe combined |
| | | | | | | | immunodeficiency) |
| 108 | 0.83 | 0.587 | 1237_at | S81914 | Hs.76095 | 8870 | immediate early |
| | | | | | | | response 3 |
| 109 | 0.82 | 0.586 | 35194_at | X53463 | Hs.2704 | 2877 | glutathione |
| | | | | | | | peroxidase 2 |
| | | | | | | | (gastrointestinal) |
| 110 | 0.82 | 0.586 | 36650 at | D13639 | Hs.75586 | 894 | cyclin D2 |
| 111 | 0.82 | 0.586 | 2075_s_at | L36719 | Hs.180533 | 5606 | mitogen-activated |
| | | | | | | | protein kinase |
| | | | | | | | kinase 3 |
| 112 | 0.82 | 0.586 | 40182_s_at | AF055027 | Hs.143696 | 10498 | coactivator- |
| | | | | | | | associated arginine |
| | | | | | | | methyltransferase-1 |
| 113 | 0.82 | 0.586 | 786_at | X06745 | Hs.267289 | 5422 | polymerase (DNA |
| | | | | | | | directed), alpha |
| 114 | 0.82 | 0.585 | 901_g_at | L41349 | Hs.283006 | 5332 | phospholipase C, |
| | | | | | | | beta 4 |
| 115 | 0.82 | 0.585 | 41200_at | Z22555 | Hs.180616 | 949 | CD36 antigen |
| | | | | | | | (collagen type I |
| | | | | | | | receptor, |
| | | | | | | | thrombospondin |
| | | | | | | | receptor)-like 1 |
| 116 | 0.82 | 0.585 | 39339_at | AB018335 | Hs.119387 | 9725 | KIAA0792 gene |
| | | | | | | | product |
| 117 | 0.81 | 0.584 | 41355_at | N95229 | Hs.130881 | 53335 | B-cell |
| | | | | | | | CLL/lymphoma |
| | | | | | | | 11A (zinc finger |
| | | | | | | | protein) |
| 118 | 0.81 | 0.584 | 40002_r_at | AI935442 | Hs.53542 | 23230 | chorein |
| 119 | 0.81 | 0.584 | 40404_s_at | U18291 | Hs.1592 | 8881 | CDC16 (cell |
| | | | | | | | division cycle 16, S. |
| | | | | | | | cerevisiae, |
| | | | | | | | homolog) |
| 120 | 0.81 | 0.583 | 40893_at | AF058953 | Hs.182217 | 8803 | succinate-CoA |
| | | | | | | | ligase, ADP- |
| | | | | | | | forming, beta |
| | | | | | | | subunit |
| 121 | 0.8 | 0.583 | 34840_at | AI700633 | Hs.288232 | | cDNA, 3 end |
| 122 | 0.8 | 0.583 | 36123_at | D87292 | Hs.248267 | 7263 | thiosulfate |
| | | | | | | | sulfurtransferase |
| | | | | | | | (rhodanese) |
| 123 | 0.8 | 0.583 | 33248_at | H94842 | Hs.17882 | | EST |
| 124 | 0.8 | 0.582 | 34866_at | AF055029 | Hs.4988 | | clone 24711 |
| 125 | 0.8 | 0.582 | 34255_at | AF059202 | Hs.288627 | 8694 | diacylglycerol O- |
| | | | | | | | acyltransferase |
| | | | | | | | (mouse) homolog |
| 126 | 0.8 | 0.582 | 37186_s_at | U11863 | Hs.75741 | 26 | amiloride binding |
| | | | | | | | protein 1 (amine |
| | | | | | | | oxidase (copper- |
| | | | | | | | containing)) |
| 127 | 0.8 | 0.582 | 41223_at | M22760 | Hs.181028 | 9377 | cytochrome c |
| | | | | | | | oxidase subunit Va |
| 128 | 0.79 | 0.581 | 34335_at | AI765533 | Hs.30942 | 1948 | ephrin-B2 |
| 129 | 0.79 | 0.581 | 34712_at | AB023227 | Hs.23860 | 23268 | KIAA1010 protein |
| 130 | 0.79 | 0.581 | 1350_at | U02388 | Hs.101 | 8529 | cytochrome P450, |
| | | | | | | | subfamily IVF, |
| | | | | | | | polypeptide 2 |
| 131 | 0.79 | 0.580 | 34829_at | U59151 | Hs.4747 | 1736 | dyskeratosis |
| | | | | | | | congenita 1, |
| | | | | | | | dyskerin |
| 132 | 0.79 | 0.580 | 40527_at | AF000571 | Hs.156115 | 3784 | potassium voltage- |
| | | | | | | | gated channel, |
| | | | | | | | KQT-like |
| | | | | | | | subfamily, member 1 |
| 133 | 0.79 | 0.580 | 37757_at | L23959 | Hs.79353 | 7027 | transcription factor |
| | | | | | | | Dp-1 |
| 134 | 0.79 | 0.580 | 37926_at | D14520 | Hs.84728 | 688 | Kruppel-like factor |
| | | | | | | | 5 (intestinal) |
| 135 | 0.79 | 0.580 | 38048_at | D84110 | Hs.80248 | 11030 | RNA-binding |
| | | | | | | | protein gene with |
| | | | | | | | multiple splicing |
| 136 | 0.78 | 0.579 | 1562_g_at | U27193 | Hs.41688 | 1850 | dual specificity |
| | | | | | | | phosphatase 8 |
| 137 | 0.78 | 0.579 | 36059_at | AB011540 | Hs.4930 | 4038 | low density |
| | | | | | | | lipoprotein |
| | | | | | | | receptor-related |
| | | | | | | | protein 4 |
| 138 | 0.78 | 0.579 | 36580_at | AL050139 | Hs.75277 | 64795 | hypothetical protein |
| | | | | | | | FLJ13910 |
| 139 | 0.78 | 0.579 | 37263_at | U55206 | Hs.78619 | 8836 | gamma-glutamyl |
| | | | | | | | hydrolase |
| | | | | | | | (conjugase, |
| | | | | | | | folylpolygammaglut |
| | | | | | | | amyl hydrolase) |
| 140 | 0.78 | 0.579 | 38381_at | U32315 | Hs.82240 | 6809 | syntaxin 3A |
| 141 | 0.78 | 0.579 | 37534_at | Y07593 | Hs.79187 | 1525 | coxsackie virus and |
| | | | | | | | adenovirus receptor |
| 142 | 0.77 | 0.578 | 34998_at | AF059531 | Hs.152337 | 10196 | protein arginine N- |
| | | | | | | | methyltransferase |
| | | | | | | | 3 (hnRNP |
| | | | | | | | methyltransferase S. |
| | | | | | | | cerevisiae)-like 3 |
| 143 | 0.77 | 0.578 | 35492_at | AC004523 | Hs.180570 | 66002 | hypothetical protein |
| | | | | | | | similar to rat |
| | | | | | | | CYP4F1 |
| 144 | 0.77 | 0.578 | 2089_s_at | H06628 | Hs.199067 | 2065 | v-erb-b2 avian |
| | | | | | | | erythroblastic |
| | | | | | | | leukemia viral |
| | | | | | | | oncogene homolog 3 |
| 145 | 0.77 | 0.578 | 39362_r_at | AF043906 | Hs.121068 | 7105 | transmembrane 4 |
| | | | | | | | superfamily |
| | | | | | | | member 6 |
| 146 | 0.77 | 0.578 | 37690_at | U61263 | Hs.78880 | 10994 | ilvB (bacterial |
| | | | | | | | acetolactate |
| | | | | | | | synthase)-like |
| 147 | 0.77 | 0.577 | 35029_at | Y07828 | Hs.91096 | 11074 | ring finger protein |
| 148 | 0.77 | 0.577 | 31849_at | AB011136 | Hs.151385 | 23078 | KIAA0564 protein |
| 149 | 0.77 | 0.577 | 40333_at | U43842 | Hs.68879 | 652 | bone |
| | | | | | | | morphogenetic |
| | | | | | | | protein 4 |
| 150 | 0.77 | 0.577 | 1827_s_at | M13929U37122 | Hs.324470 | 120 | c-myc-P64 mRNA, |
| 151 | 0.76 | 0.577 | 33103_s_at | | | | initiating from |
| | | | | | | | promoter P0, |
| | | | | | | | (HLmyc2.5) |
| | | | | | | | adducin 3 (gamma) |
| 152 | 0.76 | 0.576 | 38247_at | U67058 | Hs.168102 | | Coagulation factor |
| | | | | | | | II (thrombin) |
| | | | | | | | receptor-like 1 |
| 153 | 0.76 | 0.576 | 31854_at | AF035582 | Hs.151469 | 8573 | calcium/calmodulin- |
| | | | | | | | dependent serine |
| | | | | | | | protein kinase |
| | | | | | | | (MAGUK family) |
| 154 | 0.76 | 0.576 | 35932_at | AF081507 | | | left-right |
| | | | | | | | determination, |
| | | | | | | | factor B |
| 155 | 0.76 | 0.576 | 39540_at | AF000561 | Hs.104640 | 51341 | HFV-1 inducer of |
| | | | | | | | short transcripts |
| | | | | | | | binding protein |
| 156 | 0.76 | 0.576 | 41713_at | U09848 | Hs.132390 | 7586 | zinc finger protein |
| | | | | | | | 36 (KOX 18) |
| 157 | 0.76 | 0.576 | 35444_at | AC004030 | Hs.71779 | | Cosmid F21856 |
| 158 | 0.75 | 0.576 | 39219_at | U20240 | Hs.2227 | 1054 | CCAAT/enhancer |
| | | | | | | | binding protein |
| | | | | | | | (C/EBP), gamma |
| 159 | 0.75 | 0.575 | 37672_at | Z72499 | Hs.78683 | 7874 | ubiquitin specific |
| | | | | | | | protease 7 (herpes |
| | | | | | | | virus-associated) |
| 160 | 0.75 | 0.575 | 32502_at | AL041124 | Hs.6748 | 81544 | hypothetical protein |
| | | | | | | | PP1665 |
| 161 | 0.75 | 0.574 | 37423_at | U30246 | Hs.110736 | 6558 | solute carrier family |
| | | | | | | | 12 |
| | | | | | | | (sodium/potassium/ |
| | | | | | | | chloride |
| | | | | | | | transporters), |
| | | | | | | | member 2 |
| 162 | 0.75 | 0.574 | 37720_at | M22382 | Hs.79037 | 3329 | heat shock 60 kD |
| | | | | | | | protein 1 |
| | | | | | | | (chaperonin) |
| 163 | 0.75 | 0.574 | 1445_at | AF014958 | Hs.302043 | 9034 | chemokine (C-C |
| | | | | | | | motif) receptor-like |
| | | | | | | | 2 |
| 164 | 0.75 | 0.574 | 36821_at | AL050367 | Hs.66762 | | clone |
| | | | | | | | DKFZp564A026 |
| 165 | 0.75 | 0.573 | 37188_at | X92720 | Hs.75812 | 5106 | phosphoenolpyruvate |
| | | | | | | | carboxykinase 2 |
| | | | | | | | (mitochondrial) |
| 166 | 0.75 | 0.573 | 37177_at | Y00636 | Hs.75626 | 965 | CD58 antigen, |
| | | | | | | | (lymphocyte |
| | | | | | | | function-associated |
| | | | | | | | antigen 3) |
| 167 | 0.75 | 0.573 | 31669_s_at | AF039307 | Hs.249171 | 3207 | homeo box A11 |
| 168 | 0.75 | 0.573 | 35673_at | U02082 | Hs.334 | 7984 | Rho guanine |
| | | | | | | | nucleotide |
| | | | | | | | exchange factor |
| | | | | | | | (GEF) 5 |
| 169 | 0.75 | 0.573 | 283_at | L16842 | Hs.119251 | 7384 | ubiquinol- |
| | | | | | | | cytochrome c |
| | | | | | | | reductase core |
| | | | | | | | protein I |
| 170 | 0.75 | 0.572 | 35727_at | AI249721 | Hs.39850 | 54963 | hypothetical protein |
| | | | | | | | FLJ20517 |
| 171 | 0.74 | 0.572 | 40445_at | AF017307 | Hs.166096 | 1999 | E74-like factor 3 |
| | | | | | | | (ets domain |
| | | | | | | | transcription factor, |
| | | | | | | | epithelial-specific) |
| 172 | 0.74 | 0.572 | 1943_at | X51688 | Hs.85137 | 890 | cyclin A2 |
| 173 | 0.74 | 0.572 | 39801_at | AF046889 | Hs.153357 | 8985 | procollagen-lysine, |
| | | | | | | | 2-oxoglutarate 5- |
| | | | | | | | dioxygenase 3 |
| 174 | 0.74 | 0.572 | 288_s_at | L25931 | Hs.152931 | 3930 | lamin B receptor |
| 175 | 0.74 | 0.571 | 32320_at | Z11502 | Hs.181107 | 312 | annexin A13 |
| 176 | 0.74 | 0.571 | 3750 l_at | Y07707 | Hs.119018 | 55922 | transcription factor |
| | | | | | | | NRF |
| 177 | 0.73 | 0.571 | 476_s_at | U50079 | Hs.88556 | 3065 | histone deacetylase |
| | | | | | | | 1 |
| 178 | 0.73 | 0.571 | 864_at | U07664 | | | homeo box HB9 |
| 179 | 0.73 | 0.570 | 34046_at | Z83844 | Hs.97858 | 23616 | hypothetical protein |
| | | | | | | | dJ37E16.5 |
| 180 | 0.73 | 0.570 | 1385_at | M77349 | Hs.118787 | 7045 | transforming |
| | | | | | | | growth factor, beta- |
| | | | | | | | induced, 68 kD |
| 181 | 0.73 | 0.570 | 31887_at | J04469 | Hs.153998 | 1159 | creatine kinase, |
| | | | | | | | mitochondrial 1 |
| | | | | | | | (ubiquitous) |
| 182 | 0.73 | 0.570 | 36764_at | AC004125 | Hs.7235 | 10368 | calcium channel, |
| | | | | | | | voltage-dependent, |
| | | | | | | | gamma subunit 3 |
| 183 | 0.73 | 0.570 | 35140_at | R59697 | Hs.25283 | 1024 | cyclin-dependent |
| | | | | | | | kinase 8 |
| 184 | 0.73 | 0.570 | 367_at | Z29067 | Hs.2236 | 4752 | NIMA (never in |
| | | | | | | | mitosis gene a)- |
| | | | | | | | related kinase 3 |
| 185 | 0.73 | 0.569 | 41276_at | W27641 | Hs.23964 | 10284 | sin3-associated |
| | | | | | | | polypeptide, 18 kD |
| 186 | 0.73 | 0.569 | 37562_at | L11370 | Hs.79769 | 5097 | protocadherin 1 |
| | | | | | | | (cadherin-like 1) |
| 187 | 0.73 | 0.569 | 38630_at | AL080192 | Hs.101282 | | clone |
| | | | | | | | DKFZp434B102) |
| 188 | 0.73 | 0.569 | 40123_at | D87435 | Hs.155499 | 8729 | golgi-specific |
| | | | | | | | brefeldin A |
| | | | | | | | resistance factor 1 |
| 189 | 0.73 | 0.569 | 32601_s_at | AC004382 | Hs.279832 | 55715 | small inducible |
| | | | | | | | cytokine subfamily |
| | | | | | | | A (Cys-Cys), |
| | | | | | | | member 17 |
| 190 | 0.72 | 0.569 | 33573_at | AB009426 | | | apolipoprotein B |
| | | | | | | | mRNA editing |
| | | | | | | | enzyme, catalytic |
| | | | | | | | polypeptide 1 |
| 191 | 0.72 | 0.569 | 35656_at | AJ010346 | Hs.32597 | 6049 | ring finger protein |
| | | | | | | | (C3H2C3 type) 6 |
| 192 | 0.72 | 0.569 | 39876_at | AL035252 | Hs.12330 | 955 | ectonucleoside |
| | | | | | | | triphosphate |
| | | | | | | | diphosphohydrolase |
| | | | | | | | 6 (putative |
| | | | | | | | function) |
| 193 | 0.72 | 0.569 | 2064_g_at | L20046 | Hs.48576 | 2073 | excision repair |
| | | | | | | | cross- |
| | | | | | | | complementing |
| | | | | | | | rodent repair |
| | | | | | | | deficiency, |
| | | | | | | | complementation |
| | | | | | | | group 5 (xeroderma |
| | | | | | | | pigmentosum, |
| | | | | | | | complementation |
| | | | | | | | group G (Cockayne |
| | | | | | | | syndrome)) |
| 194 | 0.72 | 0.569 | 40067_at | M82882 | Hs.154365 | 1997 | E74-like factor 1 |
| | | | | | | | (ets domain |
| | | | | | | | transcription factor) |
| 195 | 0.72 | 0.568 | 34339_at | AB009282 | Hs.79103 | 80777 | cytochrome b5 |
| | | | | | | | outer mitochondrial |
| | | | | | | | membrane |
| | | | | | | | precursor |
| 196 | 0.72 | 0.568 | 38518_at | Y18004 | Hs.171558 | 10389 | sex comb on midleg |
| | | | | | | | (Drosophila)-like 2 |
| 197 | 0.71 | 0.567 | 37809_at | U41813 | Hs.127428 | 3205 | homeo box A9 |
| 198 | 0.71 | 0.567 | 36613_at | U09585 | Hs.315177 | 7866 | interferon-related |
| | | | | | | | developmental |
| | | | | | | | regulator 2 |
| 199 | 0.71 | 0.567 | 31324_at | U82303 | Hs.123080 | | unknown protein |
| | | | | | | | mRNA |
| 200 | 0.71 | 0.567 | 308_f_at | J03756 | Hs.65149 | 2689 | growth hormone 2 |

According to the invention, preferred markers are markers 1-30, preferably 1-20, and more preferably 1-10. Highly preferred markers are cytokeratin 20 and villin 1.

TABLE 7
C0 Markers
According to the invention, preferred markers are markers 1-30, preferably 1-20,
and more preferably 1-10.
Class: C0

| | | | | | UNIGENE | | |
|---|---|---|---|---|---|---|---|
| | | | | | (as of | | Desc |
| | | Perm | | GB/TIGR | summer | | (unigene/locuslink |
| | s2n_obs | 0.1% | non_norm_list | Identifier | 2001) | LL_num | or affy) |
| 1 | 0.81 | 0.681 | 493_at | U29171 | Hs.75852 | 1453 | casein kinase 1, delta |
| 2 | 0.8 | 0.620 | 39431_at | AJ132583 | Hs.293007 | 9520 | Aminopeptidase |
| | | | | | | | puromycin sensitive |
| 3 | 0.78 | 0.599 | 1953_at | AF024710 | Hs.73793 | 7422 | vascular endothelial |
| | | | | | | | growth factor |
| 4 | 0.75 | 0.584 | 34678_at | AL096713 | Hs.234680 | 26509 | fer-1 (C. elegans)- |
| | | | | | | | like 3 (myoferlin) |
| 5 | 0.73 | 0.570 | 32919_at | AC004010 | Hs.121520 | | BAC clone |
| | | | | | | | GS099H08 |
| 6 | 0.72 | 0.545 | 884_at | M59911 | Hs.265829 | 3675 | integrin, alpha 3 |
| | | | | | | | (antigen CD49C, |
| | | | | | | | alpha 3 subunit of |
| | | | | | | | VLA-3 receptor) |
| 7 | 0.71 | 0.531 | 38261_at | AF085692 | Hs.90786 | 8714 | ATP-binding |
| | | | | | | | cassette, sub-family |
| | | | | | | | C (CFTR/MRP), |
| | | | | | | | member 3 |
| 8 | 0.7 | 0.528 | 33889_s_at | D79985 | Hs.2491 | 9993 | DiGeorge syndrome |
| | | | | | | | critical region gene 2 |
| 9 | 0.7 | 0.524 | 31888_s_at | AF001294 | Hs.154036 | 7262 | tumor suppressing |
| | | | | | | | subtransferable |
| | | | | | | | candidate 3 |
| 10 | 0.69 | 0.522 | 38127_at | Z48199 | Hs.82109 | 6382 | syndecan 1 |
| 11 | 0.66 | 0.514 | 38132_at | M88338 | Hs.148101 | 11135 | serum constituent |
| | | | | | | | protein |
| 12 | 0.65 | 0.511 | 2017_s_at | M64349 | Hs.82932 | 893 | cyclin D1 (PRAD1: |
| | | | | | | | parathyroid |
| | | | | | | | adenomatosis 1) |
| 13 | 0.64 | 0.510 | 36101_s_at | M63978 | | | vascular endothelial |
| | | | | | | | growth factor |
| 14 | 0.64 | 0.509 | 33354_at | AA630312 | Hs.194477 | 64750 | E3 ubiquitin ligase |
| | | | | | | | SMURF2 |
| 15 | 0.64 | 0.507 | 32206_at | AB007920 | Hs.18586 | 9876 | KIAA0451 gene |
| | | | | | | | product |
| 16 | 0.61 | 0.499 | 168_at | U50196 | Hs.94382 | 132 | adenosine kinase |
| 17 | 0.61 | 0.492 | 39962_at | U59305 | Hs.44708 | 8476 | Ser-Thr protein |
| | | | | | | | kinase related to the |
| | | | | | | | myotonic dystrophy |
| | | | | | | | protein kinase |
| 18 | 0.6 | 0.489 | 33944_at | S60099 | Hs.279518 | 334 | amyloid beta (A4) |
| | | | | | | | precursor-like |
| | | | | | | | protein 2 |
| 19 | 0.6 | 0.488 | 32094_at | AB017915 | Hs.158304 | 9469 | carbohydrate |
| | | | | | | | (chondroitin |
| | | | | | | | 6/keratan) |
| | | | | | | | sulfotransferase 3 |
| 20 | 0.6 | 0.486 | 40504_at | AF001601 | Hs.169857 | 5445 | paraoxonase 2 |
| 21 | 0.59 | 0.485 | 36117_at | L13616 | Hs.740 | 5747 | PTK2 protein |
| | | | | | | | tyrosine kinase 2 |
| 22 | 0.58 | 0.480 | 34256_at | AB018356 | Hs.225939 | 8869 | sialyltransferase 9 |
| | | | | | | | (CMP- |
| | | | | | | | NeuAc: lactosylcer- |
| | | | | | | | amide alpha-2,3- |
| | | | | | | | sialyltransferase; |
| | | | | | | | GM3 synthase) |
| 23 | 0.57 | 0.477 | 35212_at | AF064801 | Hs.28285 | 11236 | patched related |
| | | | | | | | protein translocated |
| | | | | | | | in renal cancer |
| 24 | 0.57 | 0.476 | 34796_at | X63679 | Hs.4147 | 23471 | translocating chain- |
| | | | | | | | associating |
| | | | | | | | membrane protein |
| 25 | 0.56 | 0.475 | 40229_at | AJ010071 | Hs.153504 | 10040 | target of myb1 |
| | | | | | | | (chicken) homolog- |
| | | | | | | | like 1 |
| 26 | 0.55 | 0.473 | 34793_s_at | M22299 | Hs.4114 | 5358 | plastin 3 (T isoform) |
| 27 | 0.55 | 0.473 | 38643_at | W87466 | Hs.246885 | 55041 | hypothetical protein |
| | | | | | | | FLJ20783 |
| 28 | 0.55 | 0.472 | 35350_at | AB011170 | Hs.6079 | 51363 | B cell RAG |
| | | | | | | | associated protein |
| 29 | 0.55 | 0.471 | 38028_at | AL050152 | Hs.301914 | 55885 | clone |
| | | | | | | | DKFZp586K1220 |
| 30 | 0.55 | 0.471 | 1030_s_at | U07806 | Hs.317 | 7150 | topoisomerase |
| | | | | | | | (DNA) I |
| 31 | 0.54 | 0.469 | 37741_at | M77836 | Hs.79217 | 5831 | pyrroline-5- |
| | | | | | | | carboxylate |
| | | | | | | | reductase 1 |
| 32 | 0.54 | 0.469 | 35294_at | M25077 | Hs.554 | 6738 | Sjogren syndrome |
| | | | | | | | antigen A2 (60 kD, |
| | | | | | | | ribonucleoprotein |
| | | | | | | | autoantigen SS- |
| | | | | | | | A/Ro) |
| 33 | 0.53 | 0.468 | 38306_at | AA477576 | Hs.94631 | 10565 | brefeldin A-inhibited |
| | | | | | | | guanine nucleotide- |
| | | | | | | | exchange protein 1 |
| 34 | 0.53 | 0.467 | 33128_s_at | W68521 | Hs.83393 | 1474 | cystatin E/M |
| 35 | 0.53 | 0.463 | 40471_at | Y09048 | Hs.168670 | 5824 | peroxisomal |
| | | | | | | | farnesylated protein |
| 36 | 0.52 | 0.462 | 31680_at | M55630 | | | topoisomerase I |
| | | | | | | | pseudogene 2 |
| 37 | 0.52 | 0.460 | 41140_at | U05875 | Hs.177559 | 3460 | interferon gamma |
| | | | | | | | receptor 2 |
| | | | | | | | (interferon gamma |
| | | | | | | | transducer 1) |
| 38 | 0.52 | 0.459 | 33931_at | X71973 | Hs.2706 | 2879 | glutathione |
| | | | | | | | peroxidase 4 |
| | | | | | | | (phospholipid |
| | | | | | | | hydroperoxidase) |
| 39 | 0.52 | 0.459 | 393_s_at | X90976 | Hs.129914 | 861 | runt-related |
| | | | | | | | transcription factor 1 |
| | | | | | | | (acute myeloid |
| | | | | | | | leukemia 1; aml1 |
| | | | | | | | oncogene) |
| 40 | 0.52 | 0.459 | 36036_at | J05500 | Hs.47431 | 6710 | spectrin, beta, |
| | | | | | | | erythrocytic |
| | | | | | | | (includes |
| | | | | | | | spherocytosis, |
| | | | | | | | clinical type I) |
| 41 | 0.51 | 0.459 | 39411_at | AL080156 | Hs.12813 | 25976 | DKFZP434J214 |
| | | | | | | | protein |
| 42 | 0.51 | 0.459 | 33454_at | AF016903 | Hs.273330 | 180 | agrin |
| 43 | 0.51 | 0.458 | 33121_g_at | AF045229 | Hs.82280 | 6001 | regulator of G- |
| | | | | | | | protein signalling 10 |
| 44 | 0.5 | 0.458 | 40093_at | X83425 | Hs.155048 | 4059 | Lutheran blood |
| | | | | | | | group (Auberger b |
| | | | | | | | antigen included) |
| 45 | 0.5 | 0.456 | 977_s_at | Z35402 | Hs.194657 | 999 | cadherin 1, type 1, |
| | | | | | | | E-cadherin |
| | | | | | | | (epithelial) |
| 46 | 0.5 | 0.456 | 33421_s_at | AB016247 | Hs.288031 | 6309 | sterol-C5-desaturase |
| | | | | | | | (fungal ERG3, delta- |
| | | | | | | | 5-desaturase)-like |
| 47 | 0.5 | 0.455 | 39712_at | AI541308 | Hs.14331 | 6284 | S100 calcium- |
| | | | | | | | binding protein A13 |
| 48 | 0.49 | 0.452 | 33894_at | AJ010046 | Hs.25155 | 10276 | neuroepithelial cell |
| | | | | | | | transforming gene 1 |
| 49 | 0.49 | 0.451 | 38042_at | X03674 | Hs.80206 | 2539 | glucose-6-phosphate |
| | | | | | | | dehydrogenase |
| 50 | 0.49 | 0.450 | 32715_at | N90862 | Hs.172684 | 8673 | vesicle-associated |
| | | | | | | | membrane protein 8 |
| | | | | | | | (endobrevin) |
| 51 | 0.49 | 0.448 | 41273_at | AL046940 | Hs.250723 | 79086 | hypothetical protein |
| | | | | | | | MGC2747 |
| 52 | 0.49 | 0.448 | 40303_at | U85658 | Hs.61796 | 7022 | transcription factor |
| | | | | | | | AP-2 gamma |
| | | | | | | | (activating enhancer- |
| | | | | | | | binding protein 2 |
| | | | | | | | gamma) |
| 53 | 0.49 | 0.446 | 39277_at | U60805 | Hs.238648 | 9180 | oncostatin M |
| | | | | | | | receptor |
| 54 | 0.48 | 0.446 | 35597_at | AJ000480 | Hs.7837 | 10221 | phosphoprotein |
| | | | | | | | regulated by |
| | | | | | | | mitogenic pathways |
| 55 | 0.48 | 0.444 | 38423_at | L38935 | Hs.83086 | | GT212 mRNA |
| 56 | 0.48 | 0.444 | 291_s_at | J04152 | Hs.23582 | 4070 | tumor-associated |
| | | | | | | | calcium signal |
| | | | | | | | transducer 2 |
| 57 | 0.48 | 0.444 | 34885_at | AJ002308 | Hs.5097 | 9144 | synaptogyrin 2 |
| 58 | 0.48 | 0.444 | 37001_at | M23254 | Hs.76288 | 824 | calpain 2, (m/II) |
| | | | | | | | large subunit |
| 59 | 0.48 | 0.443 | 40928_at | W26496 | Hs.187991 | 26118 | DKFZP564A122 |
| | | | | | | | protein |
| 60 | 0.48 | 0.443 | 41078_at | D63484 | Hs.98508 | 23144 | KIAA0150 protein |
| 61 | 0.47 | 0.443 | 32034_at | AF041259 | Hs.155040 | 7764 | zinc finger protein |
| | | | | | | | 217 |
| 62 | 0.47 | 0.442 | 37912_at | X80200 | Hs.8375 | 9618 | TNF receptor- |
| | | | | | | | associated factor 4 |
| 63 | 0.47 | 0.442 | 36933_at | D87953 | Hs.75789 | 10397 | N-myc downstream |
| | | | | | | | regulated |
| 64 | 0.47 | 0.442 | 35442_at | AB007958 | Hs.169431 | 57243 | KIAA0489 protein |
| 65 | 0.47 | 0.442 | 33754_at | U43203 | Hs.197764 | 7080 | thyroid transcription |
| | | | | | | | factor 1 |
| 66 | 0.47 | 0.442 | 34823_at | X60708 | Hs.44926 | 1803 | dipeptidylpeptidase |
| | | | | | | | IV (CD26, adenosine |
| | | | | | | | deaminase |
| | | | | | | | complexing protein |
| | | | | | | | 2) |
| 67 | 0.47 | 0.441 | 35276_at | AB000712 | Hs.5372 | 1364 | claudin 4 |
| 68 | 0.47 | 0.441 | 40088_at | X84373 | Hs.155017 | 8204 | nuclear receptor |
| | | | | | | | interacting protein 1 |
| 69 | 0.46 | 0.440 | 1274_s_at | L22005 | Hs.76932 | 997 | cell division cycle 34 |
| 70 | 0.46 | 0.440 | 39698_at | U51712 | Hs.13775 | 84525 | hypothetical protein |
| | | | | | | | SMAP31 |
| 71 | 0.46 | 0.440 | 37103_at | AF070610 | Hs.100543 | | clone 24505 |
| 72 | 0.46 | 0.439 | 39382_at | AB011089 | Hs.12372 | 23321 | KIAA0517 protein |
| 73 | 0.46 | 0.439 | 37360_at | U66711 | Hs.77667 | 4061 | lymphocyte antigen |
| | | | | | | | 6 complex, locus E |
| 74 | 0.46 | 0.439 | 32640_at | M24283 | Hs.168383 | 3383 | intercellular |
| | | | | | | | adhesion molecule 1 |
| | | | | | | | (CD54), human |
| | | | | | | | rhinovirus receptor |
| 75 | 0.45 | 0.438 | 38762_at | AF083255 | Hs.8765 | 11325 | RNA helicase- |
| | | | | | | | related protein |
| 76 | 0.45 | 0.438 | 39021_at | AB020684 | Hs.11217 | 23333 | KIAA0877 protein |
| 77 | 0.45 | 0.437 | 35326_at | AF004876 | Hs.5809 | 10897 | putative |
| | | | | | | | transmembrane |
| | | | | | | | protein; homolog of |
| | | | | | | | yeast Golgi |
| | | | | | | | membrane protein |
| | | | | | | | Yif1p (Yip1p- |
| | | | | | | | interacting factor) |
| 78 | 0.45 | 0.437 | 33942_s_at | AF004563 | Hs.239356 | 6812 | syntaxin binding |
| | | | | | | | protein 1 |
| 79 | 0.45 | 0.435 | 32830_g_at | X97544 | Hs.20716 | 10440 | translocase of inner |
| | | | | | | | mitochondrial |
| | | | | | | | membrane 17 (yeast) |
| | | | | | | | homolog A |
| 80 | 0.44 | 0.435 | 33448_at | AB000095 | Hs.233950 | 6692 | serine protease |
| | | | | | | | inhibitor, Kunitz |
| | | | | | | | type 1 |
| 81 | 0.44 | 0.434 | 36201_at | D13315 | Hs.75207 | 2739 | glyoxalase I |
| 82 | 0.44 | 0.434 | 2035_s_at | M55914 | Hs.284127 | 4346 | MYC promoter- |
| | | | | | | | binding protein 1 |
| 83 | 0.44 | 0.433 | 34759_at | U68494 | Hs.24385 | | hbc647 mRNA |
| | | | | | | | sequence |
| 84 | 0.44 | 0.433 | 38819_at | U33635 | Hs.90572 | 5754 | PTK7 protein |
| | | | | | | | tyrosine kinase 7 |

TABLE 8
Other Markers
Class: Other

| | | | | | UNIGENE | | |
|---|---|---|---|---|---|---|---|
| | | | | | (as of | | Desc |
| | | | | GB/TIGR | summer | | (unigene/locuslink |
| | s2n_obs | Perm 0.1% | non_norm_list | Identifier | 2001) | LL_num | or affy) |
| 1 | 0.46 | 0.436 | 608_at | M12529 | Hs.169401 | 348 | apolipoprotein E |
| 2 | 0.45 | 0.427 | 1665_s_at | HG544- | | | Endothelial Cell |
| | | | | HT544 | | | Growth Factor 1 |
| 3 | 0.45 | 0.373 | 35820_at | X62078 | | | GM2 ganglioside |
| | | | | | | | activator protein |
| 4 | 0.45 | 0.369 | 33338_at | M97936 | Hs.21486 | 6772 | transcription factor |
| | | | | | | | ISGF-3 |
| 5 | 0.44 | 0.362 | 37219_at | X72755 | Hs.77367 | 4283 | monokine induced |
| | | | | | | | by gamma interferon |
| 6 | 0.43 | 0.362 | 33956_at | AB018549 | Hs.69328 | 23643 | MD-2 protein |
| 7 | 0.42 | 0.355 | 34663_at | M28696 | Hs.278443 | 2213 | low-affinity IgG Fcreceptor |
| | | | | | | | (beta-Fc-gamma-RII) |
| 8 | 0.42 | 0.355 | 36879_at | M63193 | Hs.73946 | 1890 | endothelial cell |
| | | | | | | | growth factor 1 |
| | | | | | | | (platelet-derived) |
| 9 | 0.41 | 0.354 | 36659_at | X15525 | Hs.75589 | 53 | acid phosphatase 2, |
| | | | | | | | lysosomal |
| 10 | 0.41 | 0.353 | 37542_at | D86961 | Hs.79299 | 10184 | lipoma HMGIC |
| | | | | | | | fusion partner-like 2 |
| 11 | 0.4 | 0.351 | 33143_s_at | U81800 | Hs.85838 | 9123 | solute carrier family |
| | | | | | | | 16 (monocarboxylic |
| | | | | | | | acid transporters), |
| | | | | | | | member 3 |
| 12 | 0.4 | 0.350 | 36753_at | AF072099 | Hs.67846 | 11006 | leukocyte |
| | | | | | | | immunoglobulin-like |
| | | | | | | | receptor, subfamily |
| | | | | | | | B (with TM and |
| | | | | | | | ITIM domains), |
| | | | | | | | member 4 |
| 13 | 0.39 | 0.349 | 34342_s_at | AF052124 | Hs.313 | 6696 | secreted |
| | | | | | | | phosphoprotein 1 |
| | | | | | | | (osteopontin, bone |
| | | | | | | | sialoprotein I, early |
| | | | | | | | T-lymphocyte |
| | | | | | | | activation 1) |
| 14 | 0.38 | 0.347 | 37310_at | X02419 | Hs.77274 | 5328 | plasminogen |
| | | | | | | | activator, urokinase |
| 15 | 0.38 | 0.346 | 39008_at | M13699 | Hs.296634 | 1356 | ceruloplasmin |
| | | | | | | | (ferroxidase) |
| 16 | 0.37 | 0.344 | 35714_at | U89606 | Hs.38041 | 8566 | pyridoxal |
| | | | | | | | (pyridoxine, vitamin |
| | | | | | | | B6) kinase |
| 17 | 0.37 | 0.344 | 36661_s_at | X06882 | Hs.75627 | 929 | CD 14 antigen |
| 18 | 0.36 | 0.342 | 38077_at | X52022 | Hs.80988 | 1293 | collagen, type VI, |
| | | | | | | | alpha 3 |
| 19 | 0.36 | 0.340 | 32488_at | X14420 | Hs.119571 | 1281 | collagen, type III, |
| | | | | | | | alpha 1 (Ehlers- |
| | | | | | | | Danlos syndrome |
| | | | | | | | type IV, autosomal |
| | | | | | | | dominant) |
| 20 | 0.36 | 0.340 | 39945_at | U09278 | Hs.418 | 2191 | fibroblast activation |
| | | | | | | | protein, alpha |
| 21 | 0.36 | 0.339 | 128_at | X82153 | Hs.83942 | 1513 | cathepsin K |
| | | | | | | | (pycnodysostosis) |
| 22 | 0.36 | 0.336 | 31859_at | J05070 | Hs.151738 | 4318 | matrix |
| | | | | | | | metalloproteinase 9 |
| | | | | | | | (gelatinase B, 92 kD |
| | | | | | | | gelatinase, 92 kD |
| | | | | | | | type IV collagenase) |
| 23 | 0.36 | 0.335 | 32306_g_at | J03464 | Hs.179573 | 1278 | collagen, type I, |
| | | | | | | | alpha 2 |
| 24 | 0.35 | 0.334 | 40297_at | AC005053 | Hs.61635 | 26872 | six transmembrane |
| | | | | | | | epithelial antigen of |
| | | | | | | | the prostate |
| 25 | 0.35 | 0.333 | 771_s_at | D00749 | | | CD7 antigen (p41) |
| 26 | 0.35 | 0.331 | 40496_at | J04080 | Hs.169756 | 716 | complement |
| | | | | | | | component 1, s |
| | | | | | | | subcomponent |
| 27 | 0.35 | 0.329 | 1184_at | D45248 | Hs.179774 | 5721 | proteasome |
| | | | | | | | (prosome, |
| | | | | | | | macropain) activator |
| | | | | | | | subunit 2 (PA28 |
| | | | | | | | beta) |
| 28 | 0.34 | 0.329 | 1717_s_at | U45878 | Hs.127799 | 330 | baculoviral IAP |
| | | | | | | | repeat-containing 3 |
| 29 | 0.34 | 0.329 | 1039_s_at | U22431 | Hs.197540 | 3091 | hypoxia-inducible |
| | | | | | | | factor 1, alpha |
| | | | | | | | subunit (basic helix- |
| | | | | | | | loop-helix |
| | | | | | | | transcription factor) |
| 30 | 0.34 | 0.328 | 32193_at | AF030339 | Hs.286229 | 10154 | plexin C1 |
| 31 | 0.34 | 0.328 | 464_s_at | U72882 | Hs.50842 | 3430 | interferon-induced |
| | | | | | | | protein 35 |
| 32 | 0.34 | 0.325 | 41471_at | W72424 | Hs.112405 | 6280 | S100 calcium- |
| | | | | | | | binding protein A9 |
| | | | | | | | (calgranulin B) |
| 33 | 0.33 | 0.325 | 368_at | Z29083 | Hs.82128 | 10860 | 5T4 oncofetal |
| | | | | | | | trophoblast |
| | | | | | | | glycoprotein |
| 34 | 0.33 | 0.323 | 195_s_at | U28014 | Hs.74122 | 837 | caspase 4, apoptosis- |
| | | | | | | | related cysteine |
| | | | | | | | protease |
| 35 | 0.33 | 0.323 | 34386_at | AF072250 | Hs.35947 | 8930 | methyl-CpG binding |
| | | | | | | | domain protein 4 |
| 36 | 0.33 | 0.322 | 38631_at | M92357 | Hs.101382 | 7127 | tumor necrosis |
| | | | | | | | factor, alpha-induced |
| | | | | | | | protein 2 |
| 37 | 0.33 | 0.321 | 37220_at | M63835 | | | Fc fragment of IgG, |
| | | | | | | | high affinity Ia, |
| | | | | | | | receptor for (CD64) |
| 38 | 0.33 | 0.321 | 32700_at | M55543 | Hs.171862 | 2634 | guanylate binding |
| | | | | | | | protein 2, interferon- |
| | | | | | | | inducible |
| 39 | 0.32 | 0.320 | 32434_at | D10522 | Hs.75607 | 4082 | myristoylated |
| | | | | | | | alanine-rich protein |
| | | | | | | | kinase C substrate |
| | | | | | | | (MARCKS, 80K-L) |
| 40 | 0.32 | 0.320 | 34666_at | X07834 | Hs.318885 | 6648 | superoxide |
| | | | | | | | dismutase 2, |
| | | | | | | | mitochondrial |
| 41 | 0.32 | 0.320 | 1633_g_at | U77735 | Hs.80205 | 11040 | pim-2 oncogene |
| 42 | 0.32 | 0.319 | 39827_at | AA522530 | Hs.111244 | 54541 | hypothetical protein |
| 43 | 0.32 | 0.319 | 231_at | M55153 | Hs.8265 | 7052 | transglutaminase 2 |
| | | | | | | | (C polypeptide, |
| | | | | | | | protein-glutamine- |
| | | | | | | | gamma- |
| | | | | | | | glutamyltransferase) |
| 44 | 0.32 | 0.319 | 35474_s_at | Y15915 | Hs.172928 | 1277 | collagen, type I, |
| | | | | | | | alpha 1 |
| 45 | 0.32 | 0.318 | 40712_at | D26579 | Hs.86947 | 101 | a disintegrin and |
| | | | | | | | metalloproteinase |
| | | | | | | | domain 8 |
| 46 | 0.32 | 0.317 | 1042_at | U27185 | Hs.82547 | 5918 | retinoic acid receptor |
| | | | | | | | responder |
| | | | | | | | (tazarotene induced) 1 |
| 47 | 0.32 | 0.317 | 37922_at | L02648 | Hs.84232 | 6948 | transcobalamin II; |
| | | | | | | | macrocytic anemia |
| 48 | 0.32 | 0.316 | 35816_at | U46692 | Hs.695 | 1476 | cystatin B (stefin B) |
| 49 | 0.32 | 0.315 | 38111_at | X15998 | Hs.81800 | 1462 | chondroitin sulfate |
| | | | | | | | proteoglycan 2 |
| | | | | | | | (versican) |

TABLE 9
Group 1

| | s2n v. s2n v. | Genbank_{—} | |
|---|---|---|---|
| Rank | Feature | or_tigi | Description |
| 1 | 0.89 0.57 493_at | U29171 | casein kinase 1, delta |
| 2 | 0.80 0.53 39431_a | AJ132583 | puromycin sensitive amino- |
| 3 | 0.78 0.52 1953_at | AF024710 | peptidase vascular |
| | | | endothelial growth factor |
| | | | (VEGF) |
| 4 | 0.75 0.52 34678_at | AL096713 | fer-1 (C. elegans)-like 3 |
| | | | (myoferlin) |
| 5 | 0.74 0.51 36100_at | AF022375 | vascular endothelial growth |
| | | | factor (VEGF) |
| 6 | 0.73 0.51 32919_at | AC004010 | BAC clone GS099H08 |
| 7 | 0.72 0.50 884_at | M59911 | integrin, alpha 3 (CD49C |
| | | | antigen) |
| 8 | 0.71 0.49 38261_at | AF085692 | ATP-binding cassette, sub- |
| | | | family C (CFTR/MRP) |
| 9 | 0.70 0.49 31888_s_at | AF001294 | tumor suppressing subtrans- |
| | | | ferable condidate 3 |
| 10 | 0.69 0.48 38127_at | Z48199 | syndecan 1 |
| 11 | 0.69 0.46 33889_s_at | D79985 | DiGeorge syndrome critical |
| | | | region gene 2 |
| 12 | 0.66 0.46 38132_at | M88338 | serum constituent protein |
| 13 | 0.65 0.45 2017_s_at | M64349 | cyclin D1 (PRAD1: |
| | | | parathyroid adenomatosis |
| | | | 1) |
| 14 | 0.64 0.45 36101_s_at | M63978 | vascular endothelial growth |
| | | | factor (VEGF) |
| 15 | 0.64 0.45 33354_at | AA630312 | E3 ubiquitin ligase |
| | | | SMURF2 |
| 16 | 0.64 0.45 32206_at | AB007920 | KIAA0450 gene product |
| 17 | 0.64 0.44 1930_at | U83659 | ATP-binding cassette, sub- |
| | | | family C (CFTR/MRP) |
| 18 | 0.64 0.44 40237_at | AF035444 | tumor suppressing subtrans- |
| | | | ferable candidate 3 |
| 19 | 0.61 0.44 168_at | U50196 | Adenosine kinase |
| 20 | 0.61 0.44 39962_at | U59305 | ser-thr protein kinase |
| | | | PK428 |
| 21 | 0.60 0.44 33944_at | S60099 | Amyloid beta (A4) |
| | | | precursor-like protein 2 |
| 22 | 0.60 0.44 32094_at | AB017915 | condoroitin 6- |
| | | | sulfotransferase |
| 23 | 0.60 0.44 40504_at | AF001601 | paraoxoriase 2 |
| 24 | 0.59 0.44 36117_at | L13616 | PTK2, focal adhesion |
| | | | kinase |
| 25 | 0.59 0.44 40229_at | AJ010071 | target of myb1-like |

Class-CM

| | | Genbank | |
|---|---|---|---|
| Rank | s2n v. s2n v Feature | or tigi | Description |
| 1 | 2.29 0.84 40392 at | U51096 | caudal type homeo box |
| | | | transcription factor 2 |
| 2 | 1.99 0.64 170_at | U51096 | caudal type homeo box |
| | | | transcription factor 2 |
| 3 | 1.60 0.64 40736_at | X83228 | cadherini 17, LI cadherin |
| | | | (liver-intestine) |
| 4 | 1.55 0.63 37124_i_at | J04813 | cytochrome P450, subfamily |
| | | | IIIA (niphedipine oxidase) |
| 5 | 1.53 0.61 169_at | U51095 | caudal type homeo box |
| | | | transcription factor 1 |
| 6 | 1.48 0.60 40043_at | X71345 | serine protease, trypsinogen |
| | | | IV |
| 7 | 1.40 0.59 35644_at | AB014598 | Hephaestin |
| 8 | 1.38 0.59 32972_at | Z83819 | NADPH oxidase 1 |
| 9 | 1.38 0.59 38586_at | M10050 | fatty acid binding protein 1, |
| | | | liver |
| 10 | 1.33 0.58 39951_at | L20826 | plastin 1 (I isoform) |
| 11 | 1.30 0.57 988_at | X16354 | Carcineombryonic antigen- |
| | | | related cell adhesion molecule |
| | | | 1 |
| 12 | 1.30 0.57 1229_at | U785566 | Cisplatin resistance associated |
| 13 | 1.30 0.57 37415_at | AB018258 | ATPase, Class V, type 10B |
| 14 | 1.27 0.57 41708_at | AB028957 | KIAA1034 protein |
| 15 | 1.22 0.56 765_s_at | AB006781 | galectin 4 |
| 16 | 1.22 0.56 40694_at | X73502 | cytokeratin 20 |
| 17 | 1.20 0.56 39697_at | U26726 | hydroxysteroid (11-beta) |
| | | | dehydrogenase 2 |
| 18 | 1.20 0.56 33904_at | AB000714 | claudin 3 |
| 19 | 1.20 0.56 33559_at | U61412 | protein tyrosine kinase PTK6 |
| 20 | 1.19 0.56 41266_at | X53586 | Integrin, alpha 6 |
| 21 | 1.19 0.55 35415_at | X12901 | villin 1 |
| 22 | 1.19 0.55 36170_at | D83198 | protein expressed in thyroid |
| 23 | 1.18 0.55 37847_at | AB006955 | PDZ-73 protein |
| 24 | 1.16 0.55 34595_at | AF105424 | myosin IA |
| 25 | 1.16 0.55 37125_f_at | J04813 | cytochrome P450, subfamily |
| | | | IIIA (niphedipine oxidase) |

Class-C1

| | | Genbank_{—} | |
|---|---|---|---|
| Rank | s2n v: s2n v Feature | or_tigi | Description |
| 1 | 1.29 0.85 36457_at | U10860 | guanine monophosphate |
| | | | synthetase |
| 2 | 1.25 0.79 40117_at | D84557 | Minichromosome mainte- |
| | | | nance deficient (mis5, 6. |
| | | | Pombe) 6 |
| 3 | 1.22 0.75 37337_at | A1803447 | small nuclear ribonucleo- |
| | | | protein polypeptide G |
| 4 | 1.21 0.73 41547_at | AF047472 | BUB3 homolog |
| 5 | 1.17 0.69 1055_g_at | M87339 | replication factor C |
| 6 | 1.17 0.69 38840_s_at | L10678 | profilin 2 |
| 7 | 1.14 0.68 33839_at | AL096719 | profilin 2 |
| 8 | 1.12 0.68 38065_at | X62534 | high-mobility group protein 2 |
| 9 | 1.11 0.68 709_at | J00314 | tubulin, beta polypeptide |
| 10 | 1.09 0.67 41583_at | AC004770 | flap structure-specific |
| | | | endonuclease 1 |
| 11 | 1.07 0.67 34783_s_at | AF047473 | BUB3 homolog |
| 12 | 1.06 0.67 1824_s_at | J05614 | proliferating cell nuclear |
| | | | antigen (PCNA) |
| 13 | 1.05 0.65 40195_a: | X14850 | H2A histone family, member |
| | | | X |
| 14 | 1.05 0.65 39109_a | AB024704 | chromosome 20 open reading |
| | | | frame 1 |
| 15 | 1.05 0.65 207_at | M86752 | stress-induced-phosphoprotien |
| | | | 1 (Hsp70/Hsp90 organizing |
| | | | protein) |
| 16 | 1.04 0.65 1884_s_at | M15796 | proliferating cell nuclear |
| | | | antigen (PCNA) |
| 17 | 1.03 0.64 34763_a | AF020043 | chondroitin sulfate |
| | | | proteoglycan 6 (bamacan) |
| 18 | 1.03 0.64 572_at | M86699 | TTK protein kinase |
| 19 | 1.02 0.64 40619_a | M91670 | ubiquitin carrier protein |
| 20 | 1.00 0.63 151_s_at | V00599 | FK506-binding protein 1A |
| | | | (12 kD) |
| 21 | 1.00 0.63 1803_at | X05360 | cell division cycle 2, G1 to S |
| | | | and G2 to M |
| 22 | 0.99 0.63 1515_at | HG4074- | Rad2 |
| | | HT4344 | |
| 23 | 0.98 0.63 34791_a | X52882 | t-complex 1 |
| 24 | 0.97 0.63 40690_a | X54942 | CDC28 protein kinase 2 |
| 25 | 0.96 0.63 37686_s_at | Y09008 | uracil-DNA glycosylse |

Class-C2

| | S2n v. S2n v. | Genebank_{—} | |
|---|---|---|---|
| Rank | Feature | or_tigi | Description |
| 1 | 1.46 0.77 40035_a | AB012917 | kallikrein 11 |
| 2 | 1.28 0.65 40544_g_at | L08424 | achaete-acute comlex |
| | | | homolog-like 1 |
| 3 | 1.27 0.59 36606_a | X51405 | carboxypeptidase E |
| 4 | 1.21 0.59 31477_a | L08044 | trefoil factor 3 (Intestinal) |
| 5 | 1.19 0.58 36299_a | X02330 | calcitonin/calcitonin-related |
| | | | polypeptide |
| 6 | 1.17 0.57 40649_a | X64810 | proprotein convertase |
| | | | subtilisin/kexin type 1 |
| 7 | 1.16 0.57 40543_a | L08424 | achaete-acute complex |
| | | | homolog-like 1 |
| 8 | 1.16 0.57 442_at | X15187 | tumor rejection antigen |
| | | | (gp96)1 |
| 9 | 1.11 0.56 37897_s_at | AI985964 | trefoil factor 3 (Intestinal) |
| 10 | 1.06 056 36300_a | X15943 | calcitonin/calcitonin-related |
| | | | polypeptide |
| 11 | 1.02 0.56 39332_a | AF035316 | tubulin, beta polypeptide |
| 12 | 0.97 0.55 39756_g_at | Z93930 | X-box binding protein 1 |
| 13 | 0.96 0.54 39135_a | AB018310 | KIAA0767 protein |
| 14 | 0.95 0.54 34785_a | AB028948 | KIAA1025 protein |
| 15 | 0.92 0.53 37617_a | U90912 | KIAA1128 protein |
| 16 | 0.87 0.53 39755_a | Z93930 | X-box binding protein 1 |
| 17 | 0.85 0.53 37928_a | AA621555 | nuclear transcription factor |
| | | | Y, beta |
| 18 | 0.85 0.53 1788_s_at | U48807 | dual specificity phosphatase |
| | | | 4 |
| 19 | 0.84 0.53 35995_a | AF067656 | ZW10 Interactor |
| 20 | 0.84 0.53 37141_a | U39840 | hepatocyte nuclear factor 3, |
| | | | alpha |
| 21 | 0.83 0.53 40201_a | M76180 | dopa decarboxylase |
| 22 | 0.82 0.52 1823_g_at | HG4677- | Oncogene Ret/Ptc2 |
| | | HT5102 | |
| 23 | 0.82 0.52 35800_at | D63391 | platelet-activating factor |
| | | | acetylhydrolase |
| 24 | 0.81 0.52 1822_at | HG4677- | Oncogen Ret/Ptc2 |
| | | HT5102 | |
| 25 | 0.81 0.52 37426_at | U80736 | trinuclectide repeat |
| | | | containing 9 |

Class C3

| | | Genebank_{—} | |
|---|---|---|---|
| Rank | 52n v. 52n v Feature | or_tigi | Description |
| 1 | 1.42 0.67 37669_s_at | U16799 | Na+/K+ transporting |
| | | | ATPase |
| 2 | 1.20 0.61 36066_a: | AB020635 | KIAA0828 protein |
| 3 | 1.17 0.60 33699_a: | M18667 | pepsinogen C gene |
| 4 | 1.06 0.58 1081_at | M33764 | Ornithine decarboxylase 1 |
| 5 | 1.06 0.57 33396_a: | U12472 | Glutathione S-transferase pi |
| 6 | 1.06 0.57 34319_a: | AA131149 | S100 calcium-binding |
| | | | protein P |
| 7 | 1.04 0.56 829_s_a: | U21689 | Glutathione S-transferase |
| | | | pl |
| 8 | 1.02 0.55 37004_a: | J02761 | Pulmonary-associated |
| | | | surfactant |
| 9 | 1.02 0.55 40409_a: | U46689 | Aldehyde dehydrogenase 3 |
| | | | family |
| 10 | 1.02 0.52 32805_a: | U05861 | aldo-ketb reductase family |
| | | | 1 |
| 11 | 1.00 0.52 36203_a: | X16277 | Ornithine decarboxylase 1 |
| 12 | 0.99 0.52 33383_f-at | A1820718 | Retinoic acid receptor |
| 13 | 0.99 0.51 33052_a: | U95301 | Phospholipase A2 |
| 14 | 0.98 0.51 35207_a: | X76180 | Sodium channel, |
| | | | nonvoltage-gated 1 alpha |
| 15 | 0.98 0.51 38526_a: | U02882 | CAMP-specific |
| | | | phosphodiesterase |
| 16 | 0.97 0.51 38066_a: | M81600 | NAD(P)H-quinone |
| | | | oxireductase |
| 17 | 0.93 0.51 1882_g_at | HA4058- | Fusion activated Oncogene |
| | | HT4328 | Aml1-Evi-1 |
| 18 | .093 0.51 37779_at | Y08134 | acid sphingomyelinase-like |
| | | | phosphodiesterase |
| 19 | 0.92 0.50 38773_at | AB003151 | carbonyl reductase 1 |
| 20 | 0.90 0.50 700_s_at | HG371- | Mucin 1, Epithellial |
| | | HT26388 | |
| 21 | 0.89 0.50 35938_at | M72393 | phospholipase A2, group |
| | | | IVA |
| 22 | 0.88 0.50 38986_at | Z49835 | glucose regulated protein, |
| | | | 58 kD |
| 23 | 0.88 0.50 40685_at | U10868 | aldehyde dehydrogenase 3 |
| | | | family, member B1 |
| 24 | 0.87 0.49 41267_at | AB028972 | KIAA1049 protein |
| 25 | 0.86 0.49 34839_at | AB029027 | KIAA1104 protein |

Class NL

| | s2n v. s2n v. | Genbank_{—} | |
|---|---|---|---|
| Rank | Feature | or_tigi | Description |
| 1 | 1.97 0.61 32542_at | AF063002 | four and a half LIM |
| | | | domains 1 |
| 2 | 1.92 0.59 1815_g_at | D50683 | TGF-beta II receptor |
| 3 | 1.82 0.58 36119_at | AF070648 | clone 24651 mRNA |
| 4 | 1.75 0.57 35868_at | M91211 | advanced glycosylation end |
| | | | product-specific receptor |
| 5 | 1.71 0.56 39031_at | AA152406 | Cytochrome c oxidase |
| 6 | 1.70 0.56 37398_at | AA100961 | CD31 antgen |
| 7 | 1.70 0.56 40607_at | U97105 | Dihydropyrimidinase-like 2 |
| 8 | 1.70 0.56 40841_at | AF049910 | Transforming, acidic |
| | | | coiled-coil containing |
| | | | protein 1 |
| 9 | 1.69 0.55 40331_at | AF035819 | Macrophage receptor with |
| | | | collagenous structure |
| 10 | 1.68 0.55 38454_g_at | X15606 | Intercellular adhesion |
| | | | molecule 2 |
| 11 | 1.65 0.55 36569_at | X64559 | tetranectin (plasminogen- |
| | | | binding protein) |
| 12 | 1.63 0.55 39066_at | L38486 | Microfibrillar-associated |
| | | | protein 4 |
| 13 | 1.60 0.54 40282_s_at | M84526 | adipsin/complement factor |
| | | | D |
| 14 | 1.60 0.54 34320_at | AL050224 | polymerase I and transcript |
| | | | release factor |
| 15 | 1.60 0.54 37027_at | M80899 | AHNAK nucleoprotein |
| | | | (desmoyokin) |
| 16 | 1.58 0.54 33328_at | W28612 | EST |
| 17 | 1.58 0.54 1814_at | D50683 | TGF-beta II receptor |
| 18 | 1.58 0.54 35985_at | AB023137 | A kinase (PRKA) anchor |
| | | | protein 2 |
| 19 | 1.57 0.53 38177_at | AJ001015 | RAMP2 |
| 20 | 1.57 0.53 39775_at | X54488 | C1-Inhibitor |
| 21 | 1.57 0.53 770_at | D00632 | glutathione peroxidase 3 |
| 22 | 1.54 0.53 39760_at | AL031781 | KH domain RNA binding |
| | | | protein |
| 23 | 1.54 0.53 268_at | L34657 | platelet/endothelial cell |
| | | | adhesion molecule-1 |
| | | | (PECAM-1) |
| 24 | 1.53 0.52 33756_at | U39447 | amine oxidase (vascular |
| | | | adhesion protein 1) |
| 25 | 1.52 0.52 40419_at | X85116 | erythrocyte membrane |
| | | | protein band 7.2 (stomatin) |

Class-C5

| | | Genbank | |
|---|---|---|---|
| Rank | s2n v. s2n v Feature | or tigi | Description |
| 1 | 1.06 0.73 1411_at | D16154 | P-450c11 |
| 2 | 1.04 0.70 37021_at | X16832 | Cathepsin H |
| 3 | 1.02 0.70 534_s_at | U20391 | folate receptor 1 (adult) |
| 4 | 0.95 0.69 38394_at | D42047 | KIAA0089 protein |
| 5 | 0.94 0.67 1460_g_at | M68941 | Protein tyrosine phosphatase |
| 6 | 0.92 0.67 33331_at | U17077 | BENE protein |
| 7 | 0.91 0.65 38336_at | AB023230 | K1AA1013 protein |
| 8 | 0.89 0.65 31883_at | AF025794 | Methionine synthase reductase |
| | | | (MTRR) |
| 9 | 0.88 0.65 35016_at | M13560 | 1a-associated invariant |
| | | | gamma-chain |
| 10 | 0.88 0.65 37512_at | U89281 | Oxidative 3 alpha hydroxy- |
| | | | steroid dehydrogenase |
| 11 | 0.87 0.64 1629_s_at | HG3187- | Tyrosine Phosphatase 1, Non- |
| | | HT3366 | Receptor |
| 12 | 0.86 0.64 38459_g_at | L39945 | Cytochrome b5 (CYB5) gene |
| 13 | 0.86 0.64 34139_at | AL049651 | Somatostatin receptor 4 |
| 14 | 0.86 0.63 36965_at | U13616 | Ankyrin G (ANK-3) |
| 15 | 0.85 0.63 130_s_at | X82850 | Thyroid transcription factor 1 |
| 16 | 0.85 0.63 593_s_at | M34353 | v-ros avian UR2 sarcoma |
| | | | virus oncogene homolog 1 |
| 17 | 0.85 0.63 33278_at | AC004381 | SA (rat hypertension- |
| | | | associated) homolog |
| 18 | 0.85 0.63 821_s_at | U78793 | folate receptor alpha (hFR) |
| 19 | 0.82 0.63 40617_at | AC004381 | Hypothetical protein |
| | | | FLJ20274 |
| 20 | 0.82 0.63 35792_at | U67963 | Lysophospholipase-like |
| 21 | 0.80 0.63 38785_at | X52228 | mucin 1, transmembrane |
| 22 | 0.80 0.63 33967_at | M31525 | major histocompatibility |
| | | | complex, class II |
| 23 | 0.80 0.63 34198_at | U12128 | APO-1/CD95 (Fas)-associated |
| | | | phosphatase |
| 24 | 0.80 0.62 33584_at | U35146 | CDC2-related kinase |
| 25 | 0.80 0.62 33249_at | M16801 | Nuclear receptor subfamily 3, |
| | | | group C, member 2 |

The invention may be embodied in other specific forms without departing from the spirit or essential characteristics thereof. The foregoing embodiments are therefore to be considered in all respects illustrative rather then limiting on the invention described herein. Scope of the invention is thus indicated by the appended claims rather than by the foregoing description, and all changes which come within the meaning and range of equivalency of the claims are intended to be embraced therein.

Each of the patent documents and scientific publications disclosed hereinabove is incorporated by reference herein in its entirety.

1. A method for classifying lung carcinomas on the basis of gene expression, the method comprising the steps of:
a) assaying an expression level for each of a plurality of genes in a plurality of lung carcinoma samples; and,
b) performing a clustering analysis on the expression levels of step a),
thereby identifying classes of lung carcinomas on the basis of gene expression.

2. The method of claim 1, wherein said clustering analysis is selected from the group consisting of hierarchical clustering and probabilistic clustering.

3. A method for diagnosing a type of lung carcinoma, the method comprising the steps of:
a) assaying an expression level for each of a predetermined number of markers of lung carcinoma in a lung carcinoma sample; and,
b) identifying said lung carcinoma as a predetermined type of lung carcinoma if at least one of said expression levels is greater than a reference expression level.

4. The method of claim 3, wherein said predetermined number is between 2 and 50.

5. The method of claim 3, wherein said predetermined number is greater than 50.

6. The method of claim 4 or 5, wherein said markers of lung carcinoma are markers of at least two different types of lung carcinoma.

7. The method of claim 3, wherein said type of lung carcinoma is selected from the group consisting of metastatic cancers of non-lung origin, small cell lung carcinomas and non-small cell lung carcinomas.

8. The method of claim 7, wherein said non-small cell lung carcinoma is selected from the group consisting of adenocarcinomas, squamous cell carcinomas, and large cell carcinomas.

9. The method of claim 8, wherein said adenocarcinomas are selected from the group consisting of classes C1, C2, C3, and C4.

10. The method of claim 3, wherein said markers are selected from the group consisting of the genes shown in Tables 1-4.

11. The method of claim 10, wherein said markers are selected from the group consisting of kallikrein 11, achaete-scute complex (Drosophila) homolog-like 1, carboxypeptidase E, trefoil factor 3 (intestinal), calcitonin/calcitonin-related polypeptide alpha, proprotein convertase, dual specificity phosphatase 4, and dopa decarboxylase.

12. The method of claim 3, further comprising the step of providing a prognosis for a patient based on the identification of the type of lung carcinoma.

13. The method of claim 3, further comprising the step of recommending a treatment for a patient based on the identification of the type of lung carcinoma.

14. The method of claim 13, wherein said treatment is tailored to the type of lung carcinoma.

15. A method for detecting lung carcinoma in a patient, the method comprising the steps of:
a) assaying an expression level for a predetermined number of markers for lung carcinoma in a patient sample; and,
b) detecting the presence of a lung carcinoma if at least one of said expression levels is greater than a predetermined reference level.

16. The method of claim 15, wherein said predetermined number is between 2 and 50.

17. The method of claim 15, wherein said predetermined number is greater than 50.

18. The method of claim 15 or 16, wherein said markers of lung carcinoma are markers of at least two different types of lung carcinoma.

19. The method of claim 15, wherein said type of lung carcinoma is selected from the group consisting of metastatic cancers of non-lung origin, small cell lung carcinomas and non-small cell lung carcinomas.

20. The method of claim 19, wherein said non-small cell lung carcinoma is selected from the group consisting of adenocarcinomas, squamous cell carcinomas, and large cell carcinomas.

21. The method of claim 20, wherein said adenocarcinomas are selected from the group consisting of classes C1, C2, C3, and C4.

22. The method of claim 15, wherein said gene is selected from the group consisting of the genes shown in Tables 1-4.

23. The method of claim 22, wherein said markers are selected from the group consisting of kallikrein 11, achaete-scute complex (Drosophila) homolog-like 1, carboxypeptidase E, trefoil factor 3 (intestinal), calcitonin/calcitonin-related polypeptide alpha, proprotein convertase, dual specificity phosphatase 4, and dopa decarboxylase.

24. The method of claim 15, further comprising the step of providing a prognosis for a patient based on the identification of the type of lung carcinoma.

25. The method of claim 15, further comprising the step of recommending a treatment for a patient based on the identification of the type of lung carcinoma.

26. The method of claim 25, wherein said treatment is tailored to the type of lung carcinoma.

27. A diagnostic array comprising:
a) a solid support; and
b) a plurality of diagnostic agents coupled to said solid support, wherein each of said agents is used to assay the expression level of a specific marker of lung carcinoma.

28. The array of claim 27, wherein each of said diagnostic agents is selected from the group consisting of PNA, DNA, and RNA molecules that specifically hybridize to a transcript from a marker of lung carcinoma.

29. The array of claim 27, wherein each of said diagnostic agents is an antibody that specifically binds to a protein expression product of a marker of lung carcinoma.

30. The array of claim 28 or 29, wherein said marker of lung carcinoma is a gene selected from the group consisting of the genes shown in Tables 1-4.

31. The array of claim 30, wherein said lung carcinoma is an adenocarcinoma, and said marker is selected from the group consisting of kallikrein 11, achaete-scute complex (Drosophila) homolog-like 1, carboxypeptidase E, trefoil factor 3 (intestinal), calcitonin/calcitonin-related polypeptide alpha, proprotein convertase, dual specificity phosphatase 4, and dopa decarboxylase.

32. A diagnostic array consisting of:
a) a solid support; and
b) a plurality of diagnostic agents coupled to said solid support, wherein each of said agents is used to assay the expression level of a specific marker of lung carcinoma.

33. The array of claim 27 or 32, wherein said plurality comprises diagnostic agents characteristic of at least two types of lung carcinoma.

34. A system for maintaining lung cancer marker expression levels, the system comprising a memory device comprising a reference expression level for at least one marker of lung carcinoma.

35. The system of claim 34 further comprising a reference expression level for at least one marker of normal lung.

36. The system of claim 34, wherein each marker is selected from the group consisting of the genes shown in Tables 1-4.

37. The system of claim 35, wherein each marker is selected from the group consisting of kallikrein 11, achaete-scute complex (Drosophila) homolog-like 1, carboxypeptidase E, trefoil factor 3 (intestinal), calcitonin/calcitonin-related polypeptide alpha, proprotein convertase, dual specificity phosphatase 4, and dopa decarboxylase.

38. The system of claim 35, wherein said memory device is selected from the group consisting of tapes, discs, RAM, ROM, and CDROM.

39. A computer disk comprising reference expression levels for a plurality of markers of lung carcinoma.

40. A computer disk comprising a plurality of markers of lung carcinoma.

41. A method for evaluating a drug candidate, the method comprising the steps of:
a) assaying an expression level for each of a predetermined number of lung cancer marker genes in a cell sample;
b) exposing the cell sample to a drug candidate;
c) assaying an expression level for each of the marker genes in the presence of the drug candidate; and
d) identifying a positive drug candidate as one that decreases expression of at least one of said marker genes.

42. A method for monitoring drug treatment of a patient with lung cancer, the method comprising the steps of:
a) administering a drug to a patient with lung cancer; and
b) assaying the expression level of a predetermined number marker genes, wherein the expression level of the marker genes is an indicator of the disease status of the patient.

43. A method for classifying a lung carcinoma, the method comprising the steps of:
a) assaying a gene expression profile of a lung carcinoma sample;
b) comparing the gene expression profile of step a) with a reference expression profile characteristic of a known lung carcinoma type; and
c) assigning the lung carcinoma sample to a known lung carcinoma type based on the comparison of step b).
